(19) United States
(12) Patent Application Publication
(10) Pub. No.: US 2003/0087239 A1
(43) Pub. Date: May 8, 2003

(54) Target activated nucleic acid biosensor and methods of using same

(76) Inventors: Marty Stanton, Stow, MA (US); David Epstein, Belmont, MA (US); Nobuko Hamaguchi, Framingham, MA (US)

Correspondence Address: MINTZ, LEVIN, COHN, FERRIS,, GLOVSKY AND POPEO, P.C., One Financial Center, Boston, MA, 02111, US

(21) Appl. No.: 09/952,680

(22) Filed: Sep. 13, 2001

Related U.S. Application Data
(60) Provisional application No. 60/232,454, filed on Sep. 13, 2000.

(51) Int. Cl. C12Q 1/68; C07H 21/04

(52) U.S. Cl. 435/6; 536/24.3

(57) ABSTRACT

Methods for engineering a target activated biosensor are provided. Biosensors comprise a plurality of nucleic acid sensor molecules labeled with a first signaling moiety and a second signaling moiety. The nucleic acid sensor molecules recognizes target molecules which do not naturally bind to DNA. Binding of a target molecule to the sensor molecules triggers a change in the proximity of the signaling moieties which leads to a change in the optical properties of the nucleic acid sensor molecules on the biosensor. Reagents and systems for performing the method are also provided. The method is useful in diagnostic applications and drug optimization.

### RELATED APPLICATION

This application claims priority to U.S. Ser. No. 60/232,454, filed Sep. 13, 2000. The contents of this application are incorporated herein by reference in their entirety.

### FIELD OF THE INVENTION

The invention relates generally to nucleic acids and more particularly to nucleic acid sensor molecules.

### BACKGROUND OF THE INVENTION

In addition to carrying genetic information, nucleic acids can adopt complex three-dimensional structures. These three-dimensional structures are capable of specific binding to target molecules and, furthermore, of catalyzing chemical reactions. Nucleic acids will thus provide candidate detection molecules for diverse target molecules, including those which that do not naturally bind to DNA or RNA. The aptamer selection method (“In vitro selection of RNA molecules that bind specific ligands”) (Ellington and Szostak, 1990) exploits this property of nucleic acids. In aptamer selection, combinatorial libraries of oligonucleotides are screened to identify oligonucleotides, or aptamers, which bind with high affinity to pre-selected targets. Both small biomolecules (e.g., amino acids, nucleotides, NAD, S-denosyl methionine, chloramphenicol), and large biomolecules (thrombin, Ku, DNA polymerases) are effective targets for aptamers. The affinities of aptamers range from dissociation constants (K_{d}'s) of, e.g., 0.3 pM to 500 nM, with most aptamers having binding affinities in the range of 1-10 nM. Recent observations suggest that simple changes in the chemical structure of the oligonucleotides in aptamer libraries, such as 2′-fluorosubstitutions in the pyrimidines can increase these affinities by 1 to 3 orders of magnitude. An increase in the specificity of binding is also observed. For example, a modified anti-human keratinocyte growth factor (hKGF) aptamer has been shown to bind hKGF five times more tightly than rat KGF, 10⁴ to 10¹⁰ times more tightly than related growth factors, and 10¹⁰ times more tightly than an unrelated protein, such as thrombin (Pagratis, Ct al. 1997). Gold, et al. (1995, 1997), Fitzwater, Ct a!. (1996), and Eaton, et al.(1995) also report increases in the affinity and specificity of aptamer binding upon chemical modification.

Aptamer biosensors have been used to detect specific analyte molecules. For example, fluorescently labeled anti-thrombin aptamers attached to a glass surface have been used to detect the presence of thrombin proteins in a sample by detecting changes in the optical properties of the aptamers (Potyrailo, et al., 1998). In this system, binding of thrombin to the labeled aptamer is monitored by detecting fluorescent emission of the aptamer upon excitation by an evanescent field. A method of detecting binding of a ligand to an aptamer has also been described which relies on the use of fluorescence-quenching pairs whose fluorescence is sensitive to changes in secondary structure of the aptamer upon ligand binding (Stanton, et al. 2000). However, ligand-mediated changes in secondary structure were engineered into the aptamer molecule via a laborious engineering process in which four to six nucleotides were added to the 5′ end of the aptamer that was complementary to the bases at the 3′ end of the thrombin binding region. In the absence of thrombin, this structure forms a stem loop structure, while it forms a G-quartet structure in the presence of thrombin. Fluorescent and quenching groups attached to the 5′ and 3′ end signal this change.

Other nucleic acid-based detection schemes have exploited the ligand-sensitive catalytic properties of some nucleic acids, e.g., such as ribozymes. For example, Robertson and Ellington (2000) have demonstrated that a ribozyme which acquires a ligase activity upon ligand binding can be used to detect a ligand by monitoring the ligation of a small, labeled second oligonucleotide to the ribozyme. In a complementary approach, labeled allosteric ribozymes which undergo cleavage upon binding to a ligand have been used to detect ligand by monitoring the release of the label from the ribozyme (Soukup, et al., 2000, and Breaker, 1998). However, all of these detection techniques suffer from the disadvantage that the ligand-activated ribozyme is irreversibly modified in the course of generating a signal. Thus, these types of ribozymes can be used only once in an assay. Furthermore, signal generation is slow with these ribozymes and can take from one minute to one hour or more.

### SUMMARY OF THE INVENTION

The invention is based in part on the discovery of nucleic acid target activated biosensors that include nucleic acid sensor molecules whose optical properties change upon binding to a target molecule. The invention provides a target activated biosensor which can be used in multiple assays for the detection of a target molecule. The biosensor according to the invention is highly sensitive, with the ability to detect as few as 10² to 10³ molecules of a target, and is highly specific, capable of distinguishing between closely related molecules. Target molecules are detected rapidly because recognition by the nucleic sensor molecules on the biosensor leads to immediate signal generation. The biosensors are ideal for use in a clinical laboratory, affording simple, easily automated chemistry during selection and engineering, and easily automated chemistry during the detection process. The same biosensors which are used for performing diagnostic assays can be used in the development of new drugs.

Among the advantages of the invention are that it provides a biosensor reagent that can detect and signal the presence of a ligand (target) or analyte in solution, but which does so even after the ligand is removed, or after the ligand-biosensor complex dissociates. The nucleic acid sensor molecules described herein include those comprising a target molecule activation site which comprises a structure which specifically interacts with a target molecule and an optical signaling unit. The optical signaling unit includes at least one nucleotide coupled to a signaling moiety. Generation of a signal by the signaling moiety is sensitive to the conformational changes in the nucleic acid sensor molecule which occurs upon allosteric activation of the target molecule activation site by a target molecule. Allosteric activation of the nucleic acid sensor molecule can result in an irreversible change in the optical signaling properties of the optical signaling unit. Alternatively, allosteric activation of the nucleic acid sensor molecule can result in a fully reversible change in the optical signaling properties of the optical signaling unit. In one embodiment, allosteric activation arises through binding of the target molecule to the nucleic acid sensor molecule at either the target molecule activation site, at the optical signaling unit itself, or at a site comprised of part of the target molecule activation site and part of the optical signaling unit. In one embodiment of the invention, allosteric activation, by the target molecule, of the target activated biosensor results in irreversible modification of the nucleic acid sensor molecule such that a detectable optical signal is generated continuously, even after the target molecule is separated from the biosensor. Thus, in the present invention signaling does not require that target molecule remain bound to the biosensor.

In one aspect, the invention provides a nucleic acid sensor molecule that includes a target molecule activation site. The target molecule activation site includes, e.g., a structure that recognizes a target molecule, and an optical signaling unit. The optical signaling unit includes at least one nucleotide coupled to a signaling moiety; and the signaling moiety changes its optical properties (e.g., the signaling moiety may generate a signal) upon allosteric modulation of the nucleic acid sensor molecule following recognition of the target molecule.

In one embodiment, a diagnostic system is provided comprising at least one biosensor and a optical signal detector in optical communication with the biosensor. The invention further relates to methods of using the diagnostic systems in the detection of target molecules associated with disease and for the development of drugs effective against disease. Reagents and kits useful for performing the methods are also provided.

In another embodiment of the invention, the molecular switch is an affinity tag (e.g. biotin, digoxigenin) whose activity is altered by the presence of the target. In the presence of the target, the affinity tag becomes active and can drive association between the sensor molecule and the binding partner of the affinity tag (e.g., streptavidin, anti-DIG). Optical signaling mediated by the affinity tag binding partner (e.g. light generation by luciferase-conjugated streptavidin) can thus be localized to the sensor and thus provide a means for detecting the presence of the target. Changes in the local environment or relative proximity of the optical sensors associated with the nucleic acid sensor molecule can come about as a result of (1) target-induced conformational changes in the sensor, (2) chemical changes induced by the sensor molecule itself, or (3) by catalysts acting upon the sensor molecule. In the first case, target may specifically interact with the sensor to drive it from one conformation into another or from an unfolded state into a folded state. In the second case, the sensor molecule is ribozyme or deoxyribozyme whose activity is controlled by the presence of the target. In the third case, the sensor is a substrate for modification by chemical agents or catalysts (e.g. enzymes). The extent to which the sensor serves as a substrate is controlled by the presence of the target (which may act directly as the chemical agent/catalyst, as a regulator of the chemical agent/catalyst, or as a regulator of the sensor to change its susceptibility to the chemical agent/catalyst).

In one embodiment, the target activated biosensor is used to detect a target molecule through changes in the optical properties of the nucleic acid sensor molecule which occur upon binding of the nucleic acid molecule to the target molecule. The invention makes use of a molecular switch which is activated upon binding of a target to a nucleic acid molecule. In one embodiment, the molecular switch is a fluorescent label whose light emission is quenched by the proximity of a quencher. Binding of a target molecule to the nucleic acid molecule causes the label and the quencher to be separated from each other such that the fluorescent efficiency of the label dramatically increases. In another embodiment, the molecular switch is a fluorescence energy transfer (FRET) pair. Binding of a target molecule to the nucleic acid sensor molecule causes a change in the distance between donor and acceptor groups, leading to a change in the optical properties of the molecule.

In one embodiment, the nucleic acid sensor molecules are generated by selecting for nucleic acid molecules which have a target activatable catalytic activity. In this embodiment, a population of oligonucleotides is provided which are contacted with a target molecule, and a nucleic acid sensor precursor molecule is selected comprising a target molecule activation site to which the target molecule specifically binds.

In some embodiments, binding triggers a conformation change in the molecule. Each oligonucleotide in the population comprises a random sequence; and, optionally, one or more fixed sequences coupled to the random sequence. The fixed sequence can contain at least a portion of a catalytic site for catalyzing a chemical reaction (the remaining portion can be supplied by the random sequence). The catalytic site can be, but is not limited to, a ligase site, a self-cleaving site, the catalytic site of a Group I intron, the catalytic site of a Group II intron, the catalytic site of a hammerhead ribozyme, hairpin ribozymes, HDV ribozymes, L1 ligase, Bartel ligase, self-biotinylating ribozymes, and Lorsh kinase.

In one embodiment, the fixed sequence further includes sequences to aid in cloning and/or sequencing of identified molecules. Such sequences include PCR primer activation sites, promoter sequences to direct in vitro tanscription, RNA polymerase primer activation sites, restriction enzyme recognition sites, and the like. The molecule also comprises a catalytic site which comprises a sequence capable of catalyzing a chemical reaction upon binding of the target molecule to the target molecule activation site.

Nucleic acid sensor precursor molecules are identified which comprise a target activation site within the random sequence which binds specifically to a target molecule and whose catalytic activity is modifiable (e.g., activatable) by target molecule binding to the activation site. In one embodiment, the nucleic acid precursor is provide on a replicatable nucleic acid sequence (e.g., a plasmid).

In one embodiment, the nucleic acid sensor precursor molecule is converted to a nucleic acid sensor molecule by deleting or modifying at least a portion of the catalytic site, rendering the catalytic site non-functional and exposing a 3′ nucleotide couplable to a first signaling moiety and a 5′ nucleotide couplable to a second signaling moiety. Binding of the target molecule to the target molecule activation site changes the proximity of the first signaling moiety to the second signaling moiety when the first and second signaling moieties are bound to the 3′ and 5′ nucleotides, causing a change in the optical properties of the nucleic acid sensor molecule. In one embodiment, the first signaling moiety is a donor fluorophore and the second signaling moiety is a quenching molecule, and the donor fluorophore is quenched by the quenching molecule when the sensor molecule is unbound by target molecule.

In one embodiment, the nucleic acid sensor molecule comprises a target molecule activation site which comprises a structure which specifically binds to a target molecule and an optical signaling unit. The optical signaling unit comprises at least one nucleotide coupled to a signaling moiety; generation of a signal by the signaling moiety is sensitive to the conformational changes in the nucleic acid sensor molecule which occur upon binding to a target molecule. In one embodiment, the optical signaling unit comprises a first nucleotide coupled to a first signaling moiety and a second nucleotide coupled to a second signaling moiety. The first and second signaling moieties change their proximity to each other upon binding of the target molecule to the target activation site. This change in proximity generates a signal indicative of the presence of a target molecule. In another embodiment, the optical signaling unit consists of a single signaling moiety, introduced at either an internal or terminal position within the nucleic acid sensor molecule. In this embodiment, binding of the target molecule results in changes in both the conformation and physical aspect (e.g., molecular volume or mass, and thus rotational diffusion rate, etc.) of the nucleic acid sensor molecule. Conformational changes in the nucleic acid sensor molecule upon target binding will modify the chemical environment of the signaling moiety, while changes in the physical aspect of the nucleic acid sensor molecule will alter the kinetic properties of the signaling moiety. In both cases, the result will be a detectable change in the optical properties of the nucleic acid sensor molecule.

In one embodiment, a target activated biosensor is provided which comprises a plurality of nucleic acid sensor molecules. The biosensor according to the present invention can comprise nucleic acid sensor molecules which are free in solution. Alternatively, the nucleic acid sensor molecules can be bound to a substrate such as a glass, silicon, nitrocellulose, nylon, plastic or other polymer, either covalently or noncovalently, directly or indirectly (e.g., through a linker molecule). In one embodiment, the target activated biosensor comprises at least two nucleic acid based-biosensor molecules with binding specificities for different types of target molecules.

In one embodiment, a diagnostic system for the detection of at least one target molecule, is provided comprising a nucleic acid based biosensor, and a detector in optical communication with the biosensor which detects changes in the optical properties of nucleic acid sensor molecules on said biosensor. In another embodiment, the diagnostic system, further comprises a light source in optical communication with the biosensor and a processor for processing optical signals detected by the detector. In a further embodiment, the diagnostic system comprises a plurality of biosensors, each of which can have a different specificity for a target molecule.

A kit is also provided comprising standardized reagents for detecting a target molecule using the nucleic acid sensor molecules according to the invention. The kit comprises a nucleic acid sensor molecule comprising a target activation site and at least one nucleotide couplable to a signaling moiety. At least one additional reagent is provided comprising any of: reagents for attaching a first signaling moiety; reagents for attaching a second signaling moiety, control target molecules, and appropriate buffers for analyte detection. In a further embodiment, complexes of target molecules and nucleic acid sensor molecules are provided.

In one embodiment, the target activated nucleic acid biosensors are used to detect a target molecule. A biosensor is contacted with a sample and changes in the optical properties of the biosensor are determined which can be correlated to the presence or absence of a target molecule in the sample. In one embodiment, the target molecule is associated with a pathological condition or a genetic alteration. In another embodiment, a plurality of different target molecules are detected. In still a further embodiment, the detection of a plurality of different target molecules is used to create a diagnostic profile of the patient from whom the sample is obtained. The profile can be correlated with any of a wild type state, a pathological condition, or at least one genetic alteration.

In one embodiment of invention, a method for identifying a drug compound is provided, comprising identifying a profile of target molecules associated with a disease trait in a patient, administering a candidate compound to the patient, and monitoring changes in the profile. In another embodiment, the monitored profile is compared with a profile of a healthy patient or population of healthy patients, and a compound which generates a profile which is substantially similar to the profile of target molecules in the health patient(s) (based on routine statistical testing) is identified as a drug. In a further embodiment, both the profiling and the drug identification step is performed using at least one sensor molecule whose properties change upon binding to a target molecule.

In a further embodiment, a method for identifying a drug compound comprises identifying a plurality of pathway target molecules, each belonging to a pathway, monitoring the level, chemical structure, and/or activity of pathway target molecules in a patient having a disease trait, administering a candidate compound to the patient, and monitoring changes in the level, chemical structure, and/or activity of the pathway target molecules. In another embodiment, the monitored level, chemical structure, and/or activity of the pathway target molecules is compared to the level and activity of pathway target molecules in a wild type patient or patients. In a further embodiment, both the profiling of the plurality of pathway target molecules and the identification of a candidate drug is performed using at least one sensor molecule whose properties change upon binding to the pathway target molecule. Properties according to this aspect, include, e.g., optical properties, change in sequence, chemical structure, catalytic activity, or molecular weight. In a preferred embodiment, sensor molecules are target activated nucleic acid sensor molecules.

In one embodiment, samples from a treated patient are tested in vitro; however, samples can also be tested ex vivo or in vivo. Because large number of target molecules can be monitored simultaneously, the method provides a way to assess the affects of compounds on multiple drug targets simultaneously, allowing identification of the most sensitive drug targets associated with a particular trait (e.g., a disease or a genetic alteration).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a flow diagram showing a method for selecting nucleic acid sensor precursor molecules having a target molecule activatable ligase activity according to one embodiment.

FIGS. 2A and B show a nucleic acid sensor precursor molecule according to one embodiment, in which the catalytic site includes a ligase site. FIG. 2A shows the conformation of the target molecule bound form of the nucleic acid sensor precursor molecule. FIG. 2B shows the conformation of the non-target bound form of the nucleic acid sensor precursor molecule.

FIGS. 3A and B show a nucleic acid sensor molecule derived from the nucleic acid sensor precursor molecule shown in FIGS. 2A and B in which first and second nucleotides are labeled with first and second signaling moieties (F and D, respectively).

FIG. 4 is a flow diagram showing a method for selecting nucleic acid sensor precursor molecules having a target molecule activatable self-cleavage activity according to one embodiment.

FIGS. 5A and B show a nucleic acid sensor precursor molecule according to one embodiment, in which the catalytic site includes a self-cleavage site which is the catalytic core of a hammerhead ribozyme. FIG. 5A shows the conformation of the target molecule bound form of the nucleic acid sensor precursor molecule. FIG. 5B shows the conformation of the non-target bound form of the nucleic acid sensor precursor molecule.

FIGS. 6A and B show a nucleic acid sensor molecule derived from the nucleic acid sensor precursor molecule shown in FIGS. 3A and B in which first and second nucleotides are labeled with first and second signaling moieties (F and D, respectively).

FIG. 7 is a schematic diagram illustrating pathway target molecules according to one 10 embodiment.

FIG. 8 is a flow chart showing steps in a drug optimization method according to one embodiment, in which nucleic acid sensor molecules are used at each step in the method.

FIG. 9A shows a nucleic acid sensor derived from an aptamer beacon specific to thrombin in both bound and unbound conformations. FIG. 9B shows protein titration data generated with the nucleic acid sensors pictured in FIG. 9A.

FIG. 10A shows an example of a self-cleaving nucleic acid sensor bound to a solid support when used in an epi-illuminated FRET detection scheme. FIG. 10B shows the same sensor in an epi-illuminated beacon configuration, with the acceptor fluorophore replaced by a quencher group. FIG. 10C shows the same sensor in an TIR-illuminated beacon configuration.

FIG. 11 shows an example of a self-ligating nucleic acid sensor bound to a solid support when used in a TIR-illuminated detection scheme where there is a signal increase upon target binding. FIG. 11B shows the same sensor in an epi-illuminated configuration, where target binding is detected by monitoring changes in the fluorescence polarization of the fluorophore bound to the substrate.

FIG. 12 illustrates the use of beads in a homogeneous assay format utilizing a self-ligating nucleic acid sensor. FIG. 12A shows the beads prior to target binding and ligation (no emission from acceptor). FIG. 12BA shows the beads after target binding and ligation (emission from acceptor detected).

FIG. 13A shows the physical basis for FP-based detection using the aptamer beacon-derived sensor with a single fluorophore and no quencher. FIG. 13B shows an image acquired with the scanning TIR/PMT detection system described in FIG. 14 of a 4-element biosensor on a glass substrate. FIG. 13C shows an FP titration curve generated with the thrombin sensor in a biological fluid of diluted human serum.

FIG. 14 shows a schematic of a previously constructed scanning detection system utilizing TIR laser evanescent wave excitation in either large area illumination/CCD imaging mode, or scanned spot/PMT imaging mode. The schematic shows how an array can be scanned and FP or fluorescence intensity data extracted.

FIGS. 15A and 15B are schematic diagrams showing the nucleotide sequence of an ERK1 riboreporter.

FIG. 16 is a schematic diagram showing the nucleotide sequence of a ppERK riboreporter.

### DETAILED DESCRIPTION OF THE INVENTION

The invention provides allosteric nucleic acid sensor molecules which change their conformation and/or activity in response to target molecule binding. The sensor molecules comprise a target activation site and a nucleotide couplable to at least one signaling moiety. Labeling the nucleic acid sensor molecules with first and second signaling moieties generates an optical signaling unit which is capable of changing its optical properties in response to the binding of a target molecule through changes in the proximity of the first and second signaling moieties. In one embodiment, a plurality of nucleic acid sensor molecules are provided, either in solution, or immobilized on a substrate, generating a target activated biosensor. In a further embodiment, a diagnostic system is provided which comprises at least one biosensor in optical communication with a optical signal detector. Methods of using the diagnostic system are also provided, as well as kits for performing the method.

In order to more clearly and concisely describe and point out the subject matter of the claimed invention, the following definitions are provided for specific terms which are used in the following written description and the appended claims.

As defined herein, a “oligonucleotide” is used interchangeably with the term “nucleic acid” and includes RNA or DNA sequences of more than one nucleotide in either single strand or double-stranded form. A “modified oligonucleotide” includes at least one residue with any of: an altered internucleotide linkage(s), altered sugar(s), altered base(s), or combinations thereof.

As defined herein, a “target molecule” is any molecule to be detected. The term “target molecule” refers to any molecule for which an aptamer exists or can be generated for, and can be naturally occurring or artificially created.

As defined herein, “a signature target molecule” is a target molecule whose expression is correlatable with a trait in an individual.

As used herein, a “diagnostic signature target molecule” is a target molecule whose expression is, by itself or in combination with other diagnostic signature target molecules, diagnostic of that trait.

As used herein, “pathway target molecules” are target molecules involved in the same pathway and whose accumulation and/or activity is dependent on other pathway target molecules, or whose accumulation and/or activity affects the accumulation and/or activity of other pathway target molecules.

As used herein, “signature pathway target molecules” refers to a plurality of target molecules whose expression/activity! and/or structural properties is diagnostic of a particular trait.

As used herein, a molecule which “naturally binds to DNA or RNA” is one which is found within a cell in an organism found in nature.

As defined herein, a “target activation site” is the portion of the three-dimensional structure of a sequence to which a target molecule specifically binds.

As used herein a “random sequence” or a “randomized sequence” is a segment of a nucleic acid having one or more regions of fully or partially random sequences. A fully random sequence is a sequence in which there is an approximately equal probability of each base (A, T, C, and G) being present at each position in the sequence. In a partially random sequences, instead of a 25% chance that an A, T, C, or G base is present at each position, there are unequal probabilities.

As defined herein, “a fixed region” is a sequence which is selected or known.

As defined herein, “amplifying” means any process or combination of process steps that increases the amount or number of copies of a molecule or class of molecules.

As defined herein, a “nucleic acid sensor precursor molecule” is a nucleic acid molecule comprising at least the portion of a sequence comprising the target activation site, and a catalytic site which catalyzes a chemical reaction, the catalytic site being activatable by binding of a target molecule to the target activation site.

As defined herein, “a nucleic acid sensor molecule” is a nucleic acid sensor precursor molecule to which has been added, or into which has been inserted, an optical signal generating unit.

As defined herein an “optical signal generating unit” is one or more sequences which change optical properties in response to a change in the conformation of a the nucleic acid sensor molecule.

As defined herein, a nucleic acid sensor precursor molecule or a nucleic acid sensor molecule which “recognizes a target molecule” is a an nucleic acid molecule which recognizes a target molecule with a much higher degree of affinity than it binds to non-target materials in a sample. The recognition event between the nucleic acid sensor precursor molecule or nucleic acid sensor molecule need not be permanent during the time in which the resulting allosteric modulation occurs. Thus, the recognition event can be transient with respect to the ensuing allosteric modulation (e.g., conformational change) of the nucleic acid precursor molecule or nucleic acid sensor molecule.

In some embodiments, the nucleic acid sensor molecule may bind, e.g., specifically bind, to a target molecule. As defined herein, “binds specifically to a target molecule” is a an nucleic acid molecule which binds to a target molecule with a much higher degree of affinity than it binds to non-target materials in a sample. The K_{d} of a nucleic acid sensor precursor molecule for its target molecule is at least about 50-fold greater than for non-target molecules in a sample.

As used herein, “profiling nucleic acid sensor molecules” are nucleic acid sensor molecules activatable by diagnostic target molecules.

As defined herein, “pathway nucleic acid sensor target molecules” are nucleic acid sensor molecules activatable by pathway target molecules.

As defined herein, a “biosensor” comprises a plurality of nucleic acid sensor molecules.

As defined herein, a “profiling biosensor” comprises a plurality of profiling nucleic acid sensor molecules which can be used to monitor or profile the expression of a plurality of different profile target molecules simultaneously.

As defined herein, “pathway profiling biosensor” comprises a plurality of pathway 10 nucleic acid sensor molecules which can be used to monitor the expression of a plurality of different pathway target molecules simultaneously.

As defined herein, an “array” or “microarray” refers to biosensor comprising a plurality of nucleic acid sensor molecules on a solid substrate.

As defined herein, a “substrate” refers to any physical supporting surface, whether rigid, flexible, solid, porous, gel-based, or of any other material or composition.

As defined herein, “ligation” is the formation of a covalent bond between substrate molecules (one of which may be the catalyst responsible for the ligation event).

1. Generating a Target Specific Nucleic Acid Sensor Molecule

Nucleic acid sensor precursor molecules are selected which have a target molecule-sensitive catalytic activity (e.g., ligation or self-cleavage) from a pool of randomized oligonucleotides. The precursor molecules have a target activation site to which the target molecule specifically binds and a catalytic site for mediating a catalytic reaction. Binding of a target molecule to the target activation site triggers a conformation change and/or change in activity in the nucleic acid sensor precursor molecule which activates the catalytic site. In one embodiment, by modifying (e.g., removing) at least a portion of the catalytic site and coupling it to an optical signal generating unit, a nucleic acid sensor molecule is generated whose optical properties change upon binding to a target molecule. In one embodiment, the pool of randomized oligonucleotides comprises the catalytic site of a ribozyme.

A. Selecting Nucleic Acid Sensor Precursor Molecules

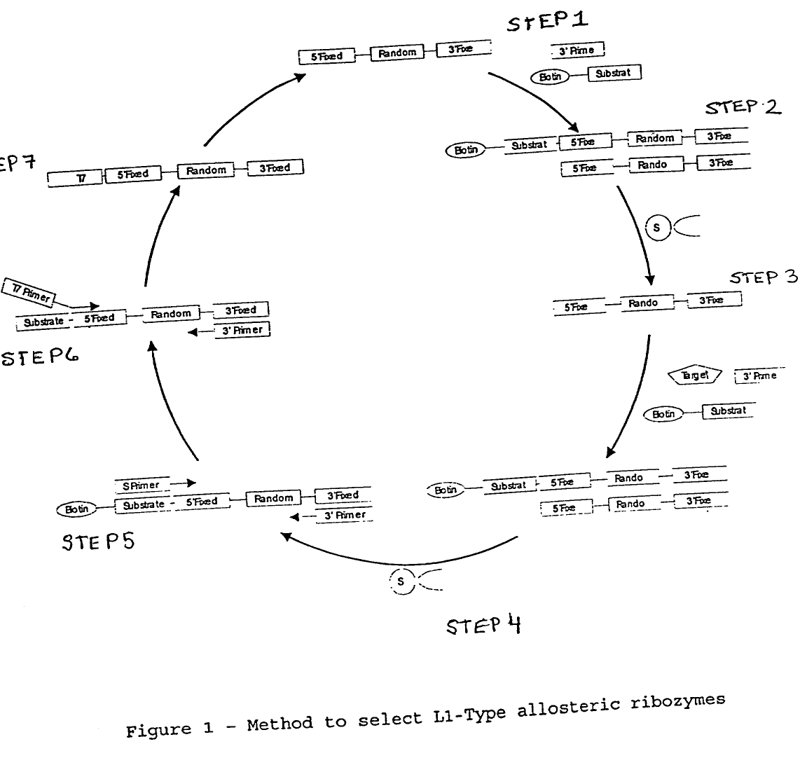

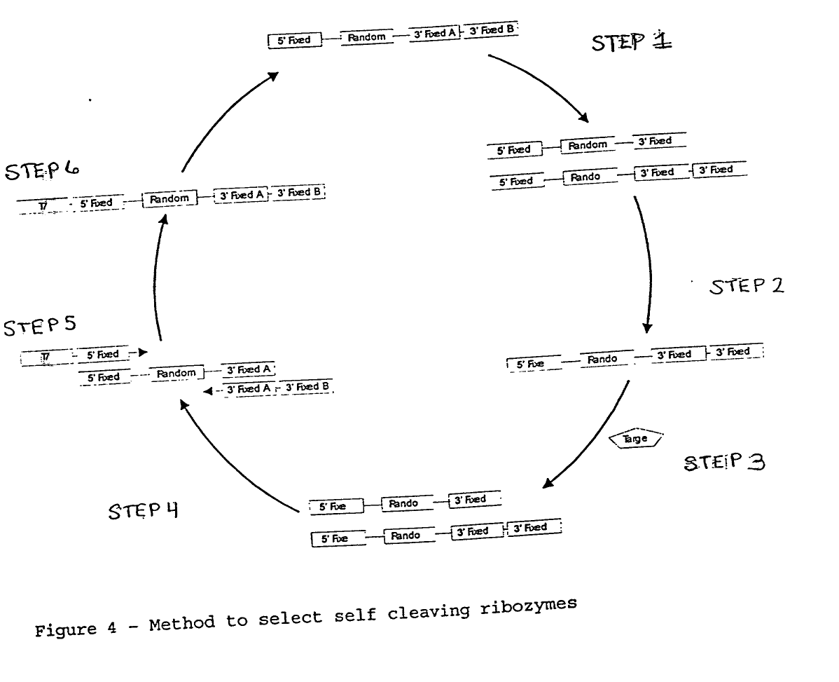

In one embodiment, a heterogeneous population of oligonucleotide molecules comprising randomized sequences is screened to identify a nucleic acid sensor precursor molecule having a catalytic activity which is modified (e.g., activated) by binding of a target molecule. Each oligonucleotide in the population comprises a random sequence and at least one fixed sequence at its 5′ and/or 3′ end. In one embodiment, the fixed sequence comprises at least a portion of a catalytic site. In the embodiments shown in FIGS. 1 and 4, the random sequence is flanked at both ends with fixed sequences.

In one embodiment, the random sequence portion of the oligonucleotide is about 15-70 (e.g., 30-40) nucleotides in length and can comprise ribonucleotides and/or deoxyribonucleotides. Random oligonucleotides can be synthesized from phosphodiester-linked nucleotides a using solid phase oligonucleotide synthesis techniques well known in the art (see, e.g., Froehler, et al., 1986a; 1986b; the entirety of which are incorporated by reference herein). Oligonucleotides can also be synthesized using solution phase methods such as triester synthesis methods (see, e.g., Sood, et al., 1977, and Hirose, et al., 1978). Typical syntheses carried out on automated DNA synthesis equipment yield 10¹⁵-10¹⁷ molecules. Sufficiently large regions of random sequence in the sequence design imply that each synthesized molecule is likely to represent a unique sequence.

To synthesize randomized sequences, mixtures of all four nucleotides are added during at each nucleotide addition step during the synthesis process, allowing for random incorporation of nucleotides. In one embodiment, random oligonucleotides comprise entirely random sequences; however, in other embodiments, random oligonucleotide can comprise stretches of nonrandom or partially random sequences. Partially random sequences can be created by adding the four nucleotides in different molar ratios at each addition step.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR2 (“amidate”), P(O)R, P(O)OR′, CO or CH2 (“formacetal”) or 3′-amine (—NH—CH2—CH2—), wherein each R or R′ is independently H or substituted or unsubstituted alkyl. Linkage groups can be attached to adjacent nucleotide through an -O-linkage or through an -N- or -S-linkage. Not all linkages in the oligonucleotide are required to be identical.

In further embodiments, the oligonucleotides comprise modified sugar groups, for example, comprising one or more of the hydroxyl groups replaced with halogen, aliphatic groups, or functionalized as ethers or amines. In one embodiment, the 2′-position of the furanose residue is substituted by any of an O-methyl, O-alkyl, O-ally!, S-alkyl, S-ally!, or halo group. Methods of synthesis of 2′-modified sugars are described in Sproat, et al., 1991; Coffen, et al., 1991; and Hobbs, et a!., 1973; the entirety of which are incorporated herein by reference. The use of 2′-fluoro-ribonucleotide oligomer molecules can increase the sensitivity of a nucleic acid sensor molecule for a target molecule by ten-to one hundred-fold over those generated using unsubstituted ribo- or deoxyribooligonucleotides (Pagratis, et al., 1997), providing additional binding interactions with a target molecule and increasing the stability of the nucleic acid sensor molecule's secondary structure(s) (Kraus, et al., 1998; Pieken, et al., 1991; Lin, et al., 1994; Jellinek, et al. 1995; Pagratis, et al., 1997).

In the embodiments shown in FIGS. 1 and 4, the random sequence portion of the oligonucleotide is flanked by at least one fixed sequence which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest. In one embodiment, the fixed sequence is approximately 50 nucleotides in length.

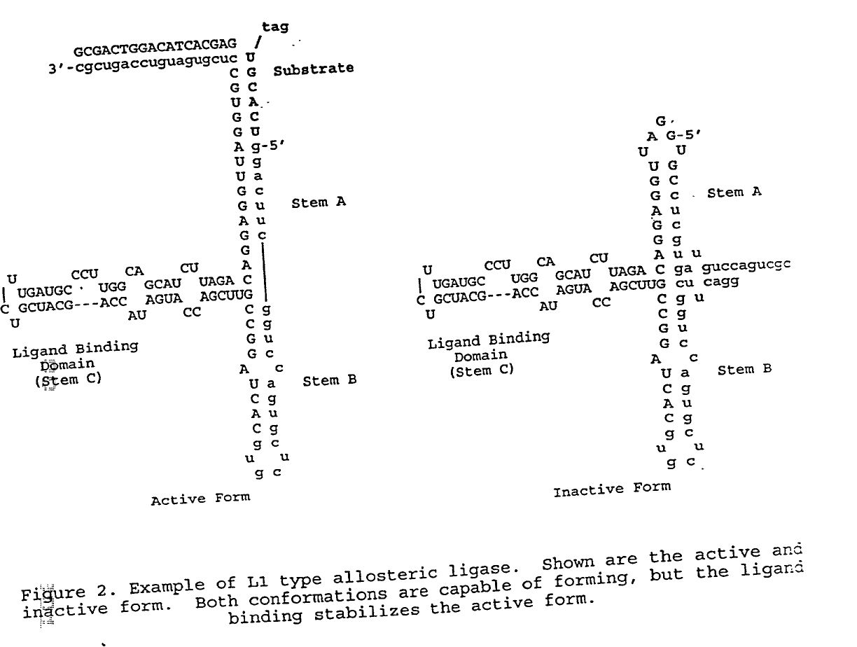

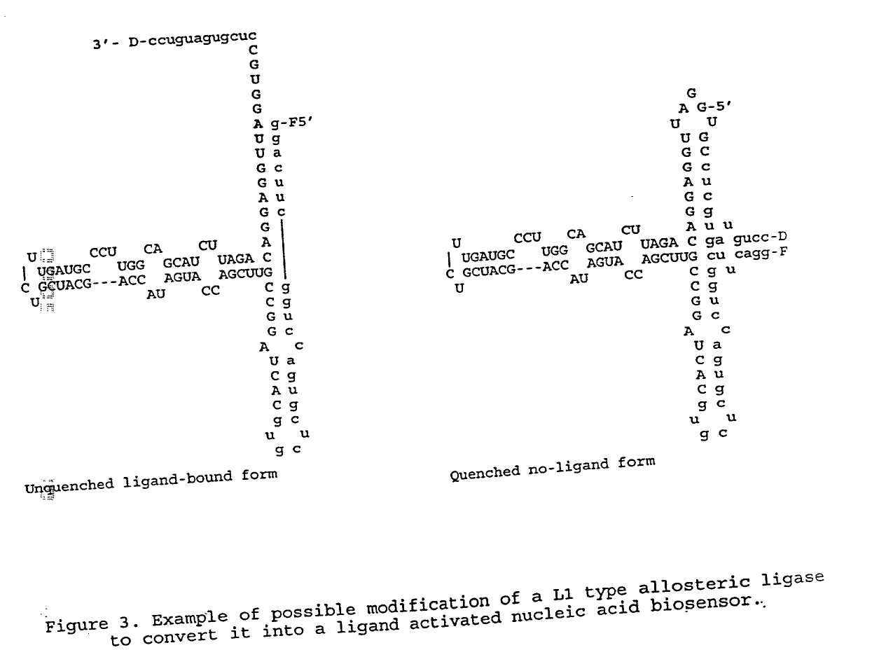

In a preferred embodiment, the fixed sequence comprises at least a portion of a catalytic site of an oligonucleotide molecule (e.g., a ribozyme) capable of catalyzing a chemical reaction. Catalytic sites are well known in the art and include, e.g. a ligase site (see FIG. 2), the catalytic sites of Group I or Group II introns (see, e.g., U.S. Pat. No. 5,780,272), the catalytic core of a hammerhead ribozyme (see, e.g., U.S. Pat. Nos. 5,767,263 and 5,700,923, and FIG. 3, herein) or a hairpin ribozyme (see, e.g., U.S. Pat. No. 5,631,359. Other catalytic sites are disclosed in U.S. Pat. No. 6,063,566, Koizumi et al., FEBS Lett. 239: 285-288 (1988), Haseloff and Gerlach, Nature 334: 585-59 (1988), Hampel and Tritz, Biochemistry 28: 4929-4933 (1989), I. Uhlenbeck, Nature, 328: 596-600 (1987), and Fedor and Uhlenbeck, Proc. Natl. Acad. Sci. USA 87: 1668-1672 (1990)).

Nucleic acid sensor precursors are generally selected in a 5 to 20 cycle procedure. In one embodiment, heterogeneity is introduced only in the initial selection stages and does not occur throughout the replicating process. FIG. 1 shows a schematic diagram in which the oligonucleotide population is screened for a nucleic acid sensor precursor molecule which comprises a target molecule activatable ligase activity.

In this embodiment, the ligation reaction involves covalent attachment of an oligonucleotide substrate to the 5′-end of the sensor precursor through formation of a phosphodiester linkage. Other ligation chemistries can form the basis for selection of sensor precursors (e.g. oligonucleotide ligation to the 3′-end, alkylation's (Wilson & Szostak), peptide bond formation (Zhang & Czech), Diels-Alder reactions to couple alkenes and dienes (Seelig & Jaschke), etc.). For some chemistries, the chemical functional groups that constitute the reactants in the ligation reaction may not naturally appear within nucleic acids. Thus, it may be necessary to synthesize an RNA pool in which one of the ligation reactants is covalently attached to each member of the pool (e.g. attaching a primary amine to the 5′-end of an RNA to enable selection for peptide bond formation).

In this embodiment, the oligonucleotide population is initially screened in a negative selection procedure to eliminate any molecules which have ligase activity even in the absence of target molecule binding. A solution of oligonucleotides (e.g., 100 pM) comprising a 5′ and 3′ fixed sequence (“5′-fixed: random: 3′-fixed”) is denatured with a 3′ primer sequence (“3′ prime”) (e.g., 200 pM) which binds to at least a portion of the 3′ fixed sequence. In one embodiment, the 5′-fixed:random:3′-fixed sequence is 5′—

(SEQ ID NO:1)
GGACUUCGGUCCAGUGCUCGUGCACUAGGCCGUUCGACC-N₃₀₋₅₀CUUA
GACAGGAGGUUAGGUGCCUCGUGAUGUCCAGUCGC-3′,

where N represents a random sequence having 30 to 50 nucleotides and the 3′ primer sequence used is 5′-GCGACTGGACATCACGAG-3′ (SEQ ID NO:2).

Ligation buffer (e.g., 30 mM Tris HCI, pH 7.4, 600 mM NaCl, 1 mM EDTA, 1% NP-40, 60 mM MgCl₂) and a tagged substrate sequence(“tag-substrate”) (e.g., Tag-UGCCACU) are added and the mixture is incubated for about 16 to about 24 hours at 25° C. in the absence of target molecule (STEP 1). Tags encompassed within the scope include, e.g., radioactive labels, fluorescent labels, a chemically reactive species such as thiophosphate the first member of a binding pair comprising a first and second binding member, each member bindable to the other (e.g., biotin, an antigen recognized by an antibody, or a tag nucleic acid sequence). The reaction is stopped by the addition of EDTA. Alternatively, the reaction can be terminated by removal of the substrate or addition of denaturants (e.g. urea, formamide).

Ligated molecules are removed from pool of selectable molecules (STEP 2), generating a population of oligonucleotides substantially free of ligated molecules (as measured by absence of the tag sequence in the solution). In the embodiment shown in FIG. 1, the tag is the first member of a binding pair (e.g., biotin) and the ligated molecules (“biotin-substrate:5′-fixed:random:3′-fixed”) are physically removed from the solution by contacting the sample to a solid support to which the second member of the binding pair is bound (“5”) (e.g., streptavidin). The eluant collected comprises a population of oligonucleotides enriched for non-ligated molecules (5′-fixed:random:3′-fixed). It should be readily apparent to those of ordinary skill in the art that this step can be repeated multiple times until the oligonucleotide population is substantially free of molecules having target-insensitive ligase activity.

This step allows for suppression of the ability of constitutively active molecules to be carried through to the next cycle of selection. Physical separation of ligated and unligated molecules is one mechanism by which this can be achieved. Alternatively, the negative selection step can be configured such that catalysis coverts active molecules to a form that blocks their ability to be either retained during the subsequent positive selection step or to be amplified for the next cycle of selection. For example, the substrate used for ligation in the negative selection step can be synthesized without a capture tag. Target-independent ligases covalently self-attach the untagged substrate during the negative selection step and are then unable to accept a tagged form of the substrate provided during the positive selection step that follows. In another embodiment, the oligonucleotide substrate provided during the negative selection step has a different sequence from that provided during the positive selection step. When PCR is carried out using a primer complementary to the positive selection substrate, only target-activated ligases will be capable of amplification.

A positive selection phase follows. In this phase, more 3′ primer and tagged substrate are added to the pool resulting from the negative selection step. Target molecules are then added to form a reacted solution and the reacted solution is incubated at 25° C. for about 2 hours (STEP 3). Target molecules encompassed within the scope include, e.g., proteins or portions thereof (e.g., receptors, antigen, antibodies, enzymes, growth factors), peptides, enzyme inhibitors, hormones, carbohydrates, polysaccharides, glycoproteins, lipids, phospholipids, metabolites, metal ions, cofactors, inhibitors, drugs, dyes, vitamins, nucleic acids, membrane structures, receptors, organelles, and viruses. Target molecules can be free in solution or can be part of a larger cellular structure (e.g., such as a receptor embedded in a cell membrane). In one embodiment, a target molecule is one which does not naturally bind to nucleic acids.

In one embodiment, nucleic acid sensor molecules are selected which are activated by target molecules comprising molecules having an identified biological activity (e.g., a known enzymatic activity, receptor activity, or a known structural role); however, in another embodiment, the biological activity of at least one of the target molecules is unknown (e.g., the target molecule is a polypeptide expressed from the open reading frame of an EST sequence, or is an uncharacterized polypeptide synthesized based on a predicted open reading frame, or is a purified or semi-purified protein whose function is unknown).

Although in one embodiment the target molecule does not naturally bind to nucleic acids, in another embodiment, the target molecule does bind in a sequence specific or non-specific manner to a nucleic acid sensor molecule. In a further embodiment, a plurality of target molecules binds to the nucleic acid sensor molecule. Selection for sensor precursors specifically responsive to a plurality of target molecules (i.e. not activated by single targets within the plurality) may be achieved by including at least two negative selection steps in which subsets of the target molecules are provided.

In still a further embodiment, nucleic acid sensor precursor molecules are selected which bind specifically to a modified target molecule but which do not bind to non-modified target molecules. Targeted modifications include, e.g., post-translational modifications of a protein, such as phosphorylation, ribosylation, methylation (Arg, Asp, N, 5, or 0-directed), prenylation (e.g., farnesyl, geranylgeranyl, and the like), acetylation, acylation, allelic variations within a protein (e.g., single amino acid changes in a protein) and cleavage sites in a protein. In another embodiment, intermediates in a chemical synthesis pathway can be targeted, as well as starting and final products. In still a further embodiment, stereochemically distinct species of a molecules can be targeted.

The reacted solution is enriched for ligated molecules (biotin-substrate: 5′-fixed :random:3′-fixed) by removing non-tagged molecules (5′-fixed:random:3′-fixed) from the solution. For example, in one embodiment, the tagged substrate comprises a biotin tag and ligated molecules are isolated by passing the reacted solution over a solid support to which streptavidin (S) is bound (STEP 4). Eluant containing non-bound, non-ligated molecules (5′-fixed:random:3′-fixed) is discarded and bound, ligated molecules (biotin-substrate: 5′-fixed:random:3′-fixed) are identified as nucleic acid sensor precursor molecules and released from the support by disrupting the binding pair interaction which enabled capture of the catalytically active molecules. For example, heating to 95° in the presence of 10 mM biotin allows release of biotin-tagged catalysts from an immobilized streptavidin support. In another embodiment, the captured catalysts remain attached to a solid support and are directly amplified (described below) while immobilized. In another embodiment, the captured catalysts remain attached to a solid support and are directly amplified (described below) while immobilized. Multiple positive selection phases can be performed (STEPS 3 and 4). In one embodiment, the stringency of each positive selection phases is increased by decreasing the incubation time by one half.

Physically removing inactive species from the pool adds stringency to the selection process. However, to the extent that the ligation reaction increases the amplification potential of the active sensor precursors, this step may be omitted. In the illustrated embodiment, for example, ligation of an oligonucleotide to the active species provides a primer binding site that enables subsequent PCR amplification using an oligonucleotide complementary to the original substrate. Unligated species do not necessarily need to be physically separated from other species because they are less likely to amplify in the absence of a covalently tethered primer binding site.

Selected nucleic acid sensor precursor molecules are amplified (or in the case of RNA molecules, first reverse transcribed, then amplified) using a substrate primer (“S primer”) (e.g., 5′-AAAAAATGCACTGGACT-3′ (SEQ ID NO:3)) which specifically binds to the substrate sequence (STEP 5). In one embodiment, amplified molecules are further amplified with a nested PCR primer that regenerates a T7 promoter (“T7 Primer”) from the 5′ fixed and the substrate sequence (STEP 6). Following transcription with T7 RNA polymerase (STEP 7), the nucleic acid sensor precursor sequence pool may be further selected and amplified to eliminate any remaining unligated sequences (5′-fixed:random:3′-fixed) by repeating STEPS 3-7. It should be obvious to those of skill in the art that in addition to PCR, and RT-PCR, any number of amplification methods can be used (either enzymatic, chemical, or replication-based, e.g., such as by cloning), either singly, or in combination. Exemplary amplification methods are disclosed in Saiki, et al., 1985; Saiki, et al., 1988; Kwoh, et al., 1989; Joyce, 1989; and Guatelli, et al., 1990. Because the 3′ primer (3′-prime) (see STEP 3 in FIG. 1) is included in the ligation mixture, selected nucleic acid sensor precursor molecules may require this sequence for activation. In cases where this is undesirable, the 3′ primer may be omitted from the mix. Alternatively, the final nucleic acid sensor molecule can be modified by attaching the 3′ primer via a short sequence loop or a chemical linker to the 3′ end of the nucleic acid sensor molecule, thereby eliminating the requirement for added primer, allowing 3′ primer sequence to self-prime the molecule.

In another embodiment, as shown in FIG. 4, an oligonucleotide population is screened for a nucleic acid sensor precursor molecule which comprises a target molecule activatable self-cleaving activity. In this embodiment, the starting population of oligonucleotide molecules comprises 5′ and 3′ fixed regions (“5′-fixed and 3′fixed A-3′ fixed B”) and at least one of the fixed regions, in this example, 3′ fixed, comprises a ribozyme catalytic core including a self cleavage site (the junction between 3′ fixed A-3′ fixed B). In one embodiment, the 5′-fixed: random:3′ fixed A-3′-fixed B molecule is GGGCGACCCUGAUGAGCCUGG-N₂₀₋₅₀-UUAGACGAAACGGUGAAAGCCGUAGGUUGCCC (SEQ ID NO:4), where N₂₀₋₅₀ is a random sequence of 20-50 nucleotides.

The population of oligonucleotide molecules comprising random olignonucleotides flanked by fixed 5′ and 3′ sequences (5′-fixed:random:3′-fixed A: 3′ fixed B) are negatively selected to remove oligonucleotides which self-cleave (i.e., 5′-fixed:random:3′-fixed-A molecules) even in the absence of target molecules. The oligonucleotide pool is incubated in reaction buffer (e.g., 50 mM Tris HCl, pH 7.5, 20 mM MgCl2) for 5 hours at 25° C., punctuated at one hour intervals by incubation at 60° C. for one minutes (STEP 1). In one embodiment, the uncleaved fraction of the oligonucleotide population (containing 5′-fixed and 3′ fixed A-3′-fixed B molecules) is purified by denaturing 10% polyacrylamide gel electrophoresis (PAGE) (STEP 2). Target molecule dependent cleavage activity is then selected in the presence of target molecules in the presence of reaction buffer by incubation at 23° C. for about 30 seconds to about five minutes (STEP 3). Cleaved molecules (5′-fixed:random:3′ fixed-A molecules) are identified as nucleic acid sensor precursor molecules and are purified by PAGE (STEP 4).

Amplification of the cleaved molecule is performed using primers which specifically 10 bind the 5′-fixed and the 3′-fixed A sequences, regenerating the T7 promoter and the 3′-fixed B site (STEP 5), and the molecule is further amplified further by RNA transcription using T7 polymerase (STEP 6). In one embodiment, the process (STEPS 1-6) is repeated until the nucleic acid sensor precursor population is reduced to about one to five unique sequences.

Alternative methods for separating cleaved from uncleaved RNAs can be used. Tags can be attached to the 3′-fixed B sequence and separation can be based upon separating tagged sequences from non-tagged sequences at STEP 4. Chromatographic procedures that separate molecules on the basis of size (e.g. gel filtration) can be used in place of electrophoresis. One end of each molecule in the RNA pool can be attached to a solid support and catalytically active molecules isolated upon release from the support as a result of cleavage. Alternate catalytic cores may be used. These alternate catalytic cores and methods using these cores are also are encompassed within the scope of the invention.

Nucleic acid sensor precursors which combine both cleavage and ligase activities in a single molecule can be isolated by using one or a combination of both of the selection strategies outlined independently above for ligases and endonucleases. For example, the hairpin ribozyme is known to catalyze cleavage followed by ligation of a second oligonucleotide substrate (Berzal-Herranz et al.). Target activated sensor precursors based on the hairpin activity can be isolated from a pool of randomized sequence RNAs prepared as described previously with a sequence of the form 5′-GGAGTTACCTAACAAGAAACAGNgaagtcaaccagNgaaacNCACGGAGACGTGNNaNattaNct ggt(N₂₀-N₅₀)GGACCTACTGAGCTGACAGTCCTGTTTGATGCATACCGAGTAAGTG-3′ where N indicates any nucleotide, lower case letters represent doped nucleotides, and uppercase letters represent fixed nucleotides. Hairpin-based sensor precursors can be isolated on the basis of effector-dependent release of the fragment 5′-GUCCUGUUUGAUGCAUACCGAGUAAGUG-3′ in the same way that hammerhead-based sensor precursors are isolated (e.g. effector-dependent increase in electrophoretic mobility or effector-dependent release from a solid support). Alternatively, sensor precursors can be selected on the basis of their ability to substitute the 3′-sequence released upon cleavage for another sequence as described in an effector-independent manner by Berzal-Heranz et al. In this scheme, the original 3′-end of the ribozyme is released in an initial cleavage event and an exogenously provided oligonucleotide substrate with a free 5′-hydroxyl is ligated back on. The newly attached 3′-end provides a primer binding site that can form the basis for preferential amplification of catalytically active molecules. Constitutively active molecules that are not activated by a provided effector can be removed from the pool by (1) separating away molecules that exhibit increased electrophoretic mobility (in the absence of an exogenous oligonucleotide substrate) in the absence of target, or (2) capturing molecules that acquire an exogenous oligonucleotide substrate (e.g. using a 3′-biotinylated substrate and captured re-ligated species on an avidin column.

Like the hairpin ribozyme, the group I intron self-splicing ribozymes combines cleavage and ligation activities to promote ligation of the exons that flank it. In the first step of group I intron-catalyzed splicing, an exogenous guanosine cofactor attacks the 5′-splice site. As a result of an intron-mediated phosphodiester exchange reaction, the 5′-exon is released coincident with attachment of the guanosine cofactor to the ribozyme. In a second chemical step, the 3′-hydroxyl at the end of the 5′-exon attacks the phosphodiester linkage between the intron and the 3′-exon, leading to ligation of the two exons and release of the intron. Group I intron-derived ribozymes can be isolated from degenerate sequence pools by selecting molecules on the basis of either one or both chemical steps, operating in either a forward or reverse direction. Effector-dependent ribozymes with potential utility as sensor precursors can be isolated by specifically enriching those molecules that fail to promote catalysis in the absence of effector but which are catalytically active in its presence. Specific examples of selection schemes follow. In each case, a pool of RNAs related in sequence to a representative group I intron (e.g. the Tetrahymena thermophila pre-rRNA intron or the phage T4 td intron) serves as the starting point for selection. Random sequence regions can be embedded within the intron at sites known to be important for proper folding and activity (e.g. substituting the P5abc domain of the Tetrahymena intron, Williams et al.).

First Step, Forward Direction

The intron is synthesized with a short 5′-exon. In the negative selection step, a guanosine cofactor is provided and constitutively active molecules undergo splicing. In the positive selection step, the sensor target is provided together with thio-GMP. Ribozymes responsive to the target undergo activated splicing and as a result acquire a unique thiophosphate at their 5′-termini. Thio-tagged ribozymes can be separated from untagged ribozymes by their specific retention on mercury gels or activated thiol agarose columns.

First Step, Reverse Direction

The method is performed as described in Green & Szostak. An intron is synthesized with a 5′-guanosine and no 5′-exon. An oligonucleotide substrate complementary to the 5′-internal guide sequence is provided during the negative selection step and constitutively active molecules ligate the substrate to their 5′-ends, releasing the original terminal guanosine. A second oligonucleotide substrate with a different 5′-sequence is provided together with target in the positive selection step. Ribozymes specifically activated by the target ligate the second oligonucleotide substrate to their 5′-ends. PCR amplification using a primer corresponding to the second substrate can be carried out to preferentially amplify target sensor precursors.

Second Step, Reverse Direction

The method is performed as described in Robertson & Joyce. The intron is synthesized with no flanking exons. During the negative selection step, pool RNAs are incubated together with a short oligonucleotide substrate under conditions which allow catalysis to proceed. During the positive selection step, a second oligonucleotide substrate with a different 3′-sequence is provided together with the sensor target. Target-dependent ribozymes are activated and catalyze ligation of the 3′-end of the second substrate. Reverse transcription carried out using a primer complementary to the 3′-end of the second substrate specifically selects target activated ribozymes for subsequent amplification.

Once nucleic acid sensor molecules are identified, they can be isolated, cloned, sequenced, and/or resynthesized using natural or modified nucleotides. Accordingly, synthesis intermediates of nucleic acid sensor molecules are also encompassed within the scope, as are replicatable sequences (e.g., plasmids) comprising nucleic acid sensor precursor molecules and nucleic acid sensor molecules.

B. Converting a Nucleic Acid Sensor Precursor Molecule to a Nucleic Acid Sensor Molecule

The nucleic acid sensor precursor molecules identified above comprise a target activation site to which a target molecule specifically binds and a catalytic site. In general, the target activation site is defined by the minimum number of nucleotides necessary to create a three-dimensional structure to which a target molecule specifically can bind, i.e., preferential binding to the target molecule in a solution comprising both target and non-target molecules. In one embodiment, the specificity of binding can be defined in terms of K_{d}. The K_{d} value can be determined directly by well-known methods (see, e.g., Caceci, et al., 1984). Alternatively, a competitive binding assay for the target molecule can be conducted with respect to control substances known to bind the target molecule. The value of the concentration at which 50% inhibition occurs (K_{d}) is, under ideal conditions, equivalent to K_{d} and sets a maximal value for the value of K_{d} (see, e.g., U.S. Pat. No. 5,756,291).

In one embodiment, the Kⱼ for the nucleic acid sensor precursor molecule with respect to the target molecule is 50-fold, e.g. 50-500 fold, or even 500-1,000,000 fold less than the Kⱼ of non-target materials in a sample.

In the examples described above, the catalytic site is a known sequence (a ligase site or a hammerhead catalytic core) and is at least a portion of either the 5′ and/or 3′ fixed region (the other portion being supplied by the random sequence), or is a complete catalytic site. However, in other embodiments, the catalytic site may be selected along with the target binding activity of oligonucleotides within the oligonucleotide pool.

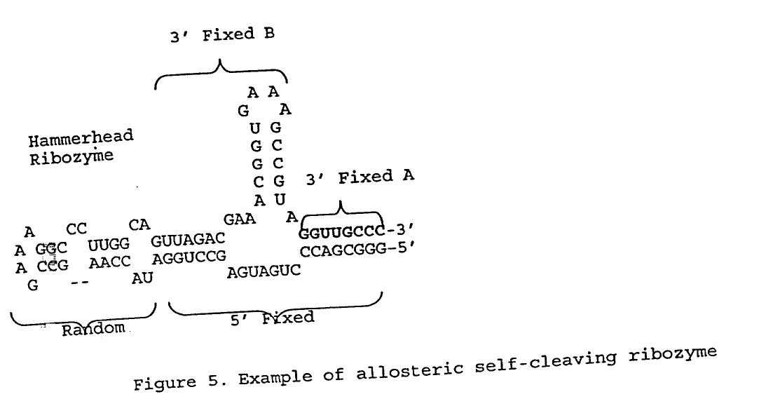

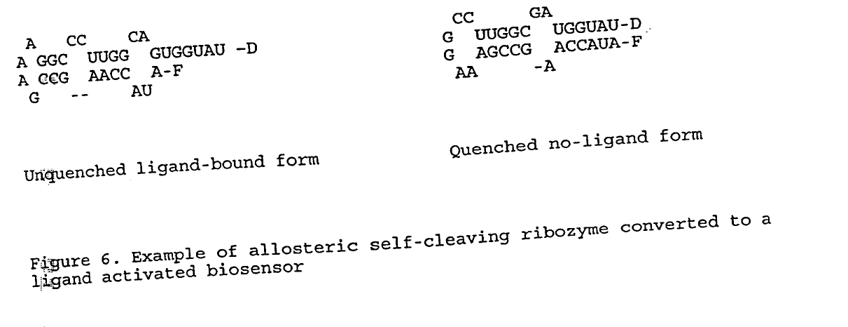

In one embodiment, in order to convert an identified nucleic acid sensor precursor molecule into a nucleic acid sensor molecule, at least a portion of the catalytic site is modified (e.g., deleted). In one embodiment, the deletion enhances the conformational stability of the nucleic acid sensor molecule in either the bound or unbound forms. In one embodiment, shown in FIGS. 6A and B, deletion of the entire catalytic domain of the nucleic acid precursor molecule shown in FIGS. 5A and B is shown to stabilize the unbound form of the nucleic acid sensor molecule. In another embodiment, the deletion may be chosen so as to take advantage of the inherent fluorescence-quenching properties of unpaired guanosine (G) residues (Walter, N. G. and Burke, J. M., “Real-time monitoring of hairpin ribozyme kinetics through base-specific quenching of fluorescein-labeled substrates”, RNA 3:392 (1997).)

In another embodiment, the ligand binding domain sequence from a previously identified nucleic acid sensor precursor molecule is incorporated into an oligonucleotide sequence that changes conformation (e.g., from a duplexed hairpin to a G-quadruplex) upon target binding. Nucleic acid sensor molecules of this type can be derived from allosteric ribozymes, such as those derived from the hammerhead, hairpin, L1 ligase, or group 1 intron ribozymes and the like, or may be derived from aptamer beacons or signaling aptamers, all of which transduce molecular recognition into a detectable signal. For example, 3′,5′-cyclic nucleotide monophosphate(cNMP)-dependent hammerhead ribozymes were reengineered into (RNA) nucleic acid sensor molecules which specifically bound to cNMP (Garretta et al., 2001). The catalytic cores for hammerhead ribozymes were removed and replaced with 5-base duplex forming sequences. The binding of these reengineered RNA sensor molecules to c-NMP was then confirmed experimentally. By adjusting the duplex length, they can be redesigned to undergo significant conformational changes. The conformational changes can be coupled to detection via FRET or simply changes in fluorescence intensity (as in the case of a molecular beacon). For example, by adding an appropriate probe on the each end of the duplex, the stabilization of duplex by target binding can be monitored with the change in fluorescence.

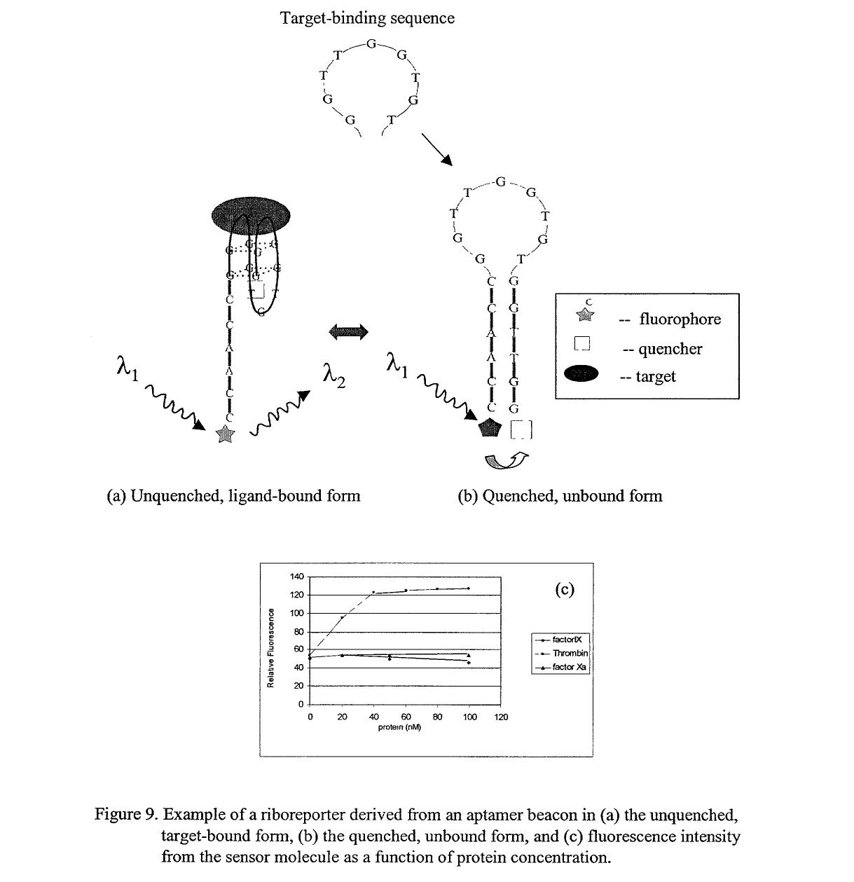

In a particular embodiment, the nucleic acid sensor molecules are made of single stranded DNA with the following sequence: 5′-CCAACGGTTGGTGTGGTTGG-3′ (SEQ ID NO:5), as shown in FIG. 9 (see Hamaguchi, et al, 2001). In addition, a fluorescein-based fluorescent label (FAM) is attached to the5′ terminus, either at the time of synthesis, or afterward. Similarly, a quenching moiety (DABCYL) is attached to the 3′ end of the nucleic acid sensor molecule. The final configuration of the nucleic acid sensor molecule is: 5′-FAM-CCAACGGTTGGTGTGGTTGG-DABCYL-3′ (SEQ ID NO:6). This particular sequence was designed to specifically bind to thrombin via the formation of a G-quadruplex. In the unbound state (see FIG. 9A), the nucleic acid sensor molecule forms a stem-loop conformation with duplex formation along the stem due to the complementarity of the nucleotides at the 3′ and 5′ ends of the molecule. In the presence of the target (here thrombin), the nucleic acid sensor molecule forms the ligand-binding G-quadruplex structure (see FIG. 9B). This conformational rearrangement results in a change in the distance between the fluorophore attached to the 5′ end and the quencher attached to the 3′ end. With the quencher spatially removed from the immediate vicinity of the fluorophore, the detected fluorescence emission intensity from the fluorophore increases sharply.

While the above experimental example is performed in solution and utilizes a cuvette-based fluorescence spectrometer, in alternative embodiments the methods are performed in microwell multiplate readers (e.g., the Packard Fusion, or the Tecan Ultra) for high-throughput solution phase measurements.

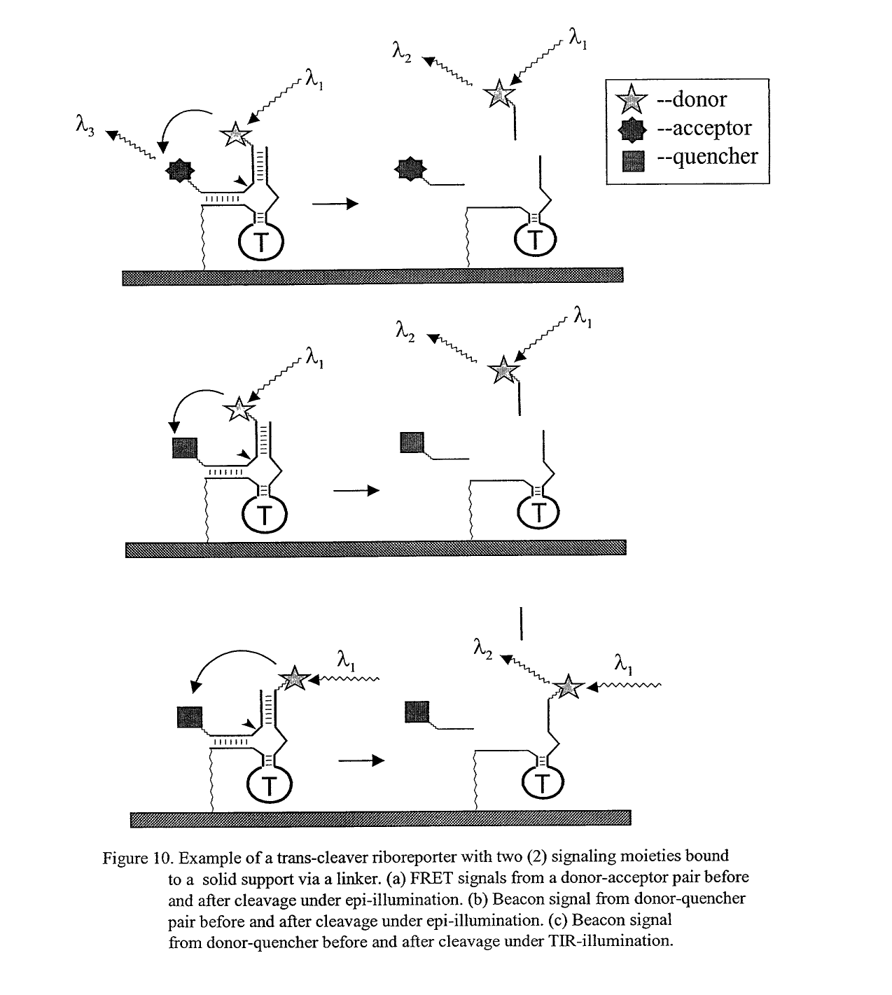

In another embodiment, a nucleic acid sensor molecule is bound to a surface by a linker attached to one end of the molecule. In a particular embodiment, depicted in FIG. 10A, the previously described (see Hamaguchi, et al, Anal. Biochem. 294, 126 (2001).) thrombin-binding sensor molecule is 5′ modified to include a 12-carbon atom linker terminated with an amino group, as well as a 3′-attached fluorescein-based fluorophore (FAM): 5′-NH2-Cl2-DABCYL-CCAACGGTTGGTGTGGTTGG-FAM-3′ (SEQ ID NO:7). This free amine group allows the sensor molecule to be attached to an aldehyde-derivatized glass surface via standard protocols for Schiff base formation and reduction. The nucleic acid sensor molecules can be bound in discrete regions or spots to form an array, or uniformly distributed to cover an extended area. In the absence of target (here thrombin), the nucleic acid sensor molecule forms a stem-loop conformation with duplex formation along the stem due to the complementarity of the nucleotides at the 3′ and 5′ ends of the molecule. In the presence of the target, the nucleic acid sensor molecule forms the ligand-binding G-quadruplex structure. This conformational rearrangement results in a change in the distance between the fluorophore attached to the 5′ end and the quencher attached to the 3′ end. With the quencher separated from the fluorophore, the detected fluorescence emission intensity from the fluorophore increases sharply. The detected increase in fluorescence intensity with target concentration can be used to detect and quantify the amount of target present in a sample solution introduced onto the surface. A sample solution could be laterally confined about the sensor surface by a coverslip, microwell, incubation chamber seal, or flowcell.

In one embodiment, after deletion of at least a portion of the catalytic site from a nucleic acid sensor precursor molecule, an optical signaling unit is either added to, or inserted within, the nucleic sensor molecule, generating a sensor molecule whose optical properties can change in response to binding to the target molecule. In one embodiment, the optical signaling unit is added by exposing at least a 5′ or 3′ nucleotide that was not previously exposed. The 5′ nucleotide or a 5′ subterminal nucleotide (e.g., an internal nucleotide) of the molecule is couplable to a first signaling moiety while the 3′ nucleotide or 3′ subterminal nucleotide is couplable to a second signaling moiety. Target binding to the nucleic acid sensor molecule alters the proximity of the 5′ and 3′ nucleotide (or subterminal nucleotides) with respect to each other, and when the first and second signaling moieties are coupled to their respective nucleotides, this change in proximity results in a target sensitive change in the optical properties of the nucleic acid sensor molecule.

Detection of changes in the optical properties of the nucleic acid sensor molecule can therefore be correlated with the presence and/or quantity of a target molecule in a sample. However, in some embodiments, it may be desirable to retain the catalytic properties of the molecule. In this embodiment, first and second signaling moieties are coupled to the 5′ terminal or subterminal sequences, and 3′-terminal and subterminal sequences respectively, of the molecule. Signaling molecules can be coupled to nucleotides which are already part of the nucleic acid sensor molecule or may be coupled to nucleotides which are inserted into the nucleic acid sensor molecule, or can be added to a nucleic acid sensor molecule as it is synthesized. Coupling chemistries to attach signaling molecules are well known in the art (see, for example, The Molecular Probes Handbook, R. Haughland). Suitable chemistries include, e.g., derivatization of the 5-position of pyrimidine bases (e.g. using 5′-amino allyl precursors), derivatization of the 5′-end (e.g. phosphoroamidites that add a primary amine to the 5′-end of chemically-synthesized oligonucleotide) or the 3′-end (e.g. periodate treatment of RNA to convert the 3′-ribose into a dialdehyde which can subsequently react with hydrazide-bearing signaling molecules).

In another embodiment, a single signaling moiety is either added to, or inserted within, the nucleic sensor molecule. In this embodiment, binding of the target molecule results in changes in both the conformation and physical aspect (e.g., molecular volume, and thus rotational diffusion rate, etc.) of the nucleic acid sensor molecule. Conformational changes in the nucleic acid sensor molecule upon target binding will modify the chemical environment of the signaling moiety, while changes in the physical aspect of the nucleic acid sensor molecule will alter the kinetic properties of the signaling moiety. In both cases, the result will be a detectable change in the optical properties of the nucleic acid sensor molecule.

In the embodiment shown in FIGS. 9A and B, the nucleic acid sensor molecule is prepared without the quencher group. The resulting molecule will have the sequence: 5′-NH2-Cl2-CCAACGGTTGGTGTGGTTGG-FAM-3′ (SEQ ID NO:8). This free amine group allows the sensor molecule to be attached to an aldehyde-derivatized glass surface via standard protocols for Schiff base formation and reduction. The nucleic acid sensor molecules can be bound in discrete regions or spots to form an array, or uniformly distributed to cover an extended area. In the absence of target (here thrombin), the nucleic acid sensor molecule will diffusionally rotate about its point of attachment to the surface at a rate characteristic of its molecular volume and mass. After target binding, the sensor-target complex will have a correspondingly larger volume and mass (thrombin mass ˜55 kD). This change in molecular volume (mass) will slow the rate of rotational diffusion, and result in a measurable change in the polarization state of the fluorescence emission from the fluorophore.

In one example of this embodiment of the invention, a single signaling moiety is attached to a portion of a ribozyme that is released as a result of catalysis (e.g. either end of a self-cleaving ribozyme or the pyrophosphate at the 5′-end of a ligase). Target-activated catalysis leads to release of the signaling moiety from the ribozyme to generate a signal correlated with the presence of the target. Release can be detected by either (1) changes in the intrinsic optical properties of the signaling moiety (e.g. decreased fluorescence polarization as the released moiety is able to tumble more freely in solution), or (2) changes in the partitioning of the signaling moiety (e.g. release of a fluorophore from a chip containing immobilized ribozymes such that the total fluorescence of the chip is reduced following washing).

In another embodiment of the invention, the nucleic acid sensor precursor is unmodified and the optical signaling unit is provided as a substrate for the ribozyme. One example of this embodiment includes a fluorescently tagged oligonucleotide which can be joined to a ribozyme with ligase activity. In a heterogeneous assay using the ligase as a sensor molecule, analyte-containing samples are incubated with the fluorescent oligonucleotide substrate and the ligase under conditions that allow the ligase to function. Following an incubation period, the ligase is separated from free oligonucleotide substrate (e.g. by capturing ligases onto a solid support on the basis of hybridization to ligase-specific sequences or by pre-immobilizing the ligases on a solid support and washing extensively).

Quantitation of the captured fluorescence signal provides a means for inferring the concentration of analyte in the sample. In a second example of this embodiment, ribozyme activity alters the fluorescence properties of a substrate without leading to its own modification. Fluorophore pairs or fluorophore/quencher pairs can be attached to nucleotides flanking either side of the cleavage site of an oligonucleotide substrate for a trans-acting endonuclease ribozyme (Jenne et al.). Target activated cleavage of the substrate leads to separation of the pair and a change in its optical properties.

In another embodiment of the invention, the sensor ribozyme and its substrates are unmodified and detection relies on catalytically-coupled changes in the ability of the ribozyme to be enzymatically amplified. In one example, a target-activated ligase is incubated together with oligonucleotide substrate and an analyte-containing sample under conditions which allow the ligase to function. Following an incubation period, the reaction is quenched and the mixture subjected to RT/PCR amplification using a primer pair that includes the oligo sequence corresponding to the ligation substrate. Amplification products can be detected by a variety of generally practiced methods (e.g. Taqman). Only those ribozymes that have self-ligated an oligonucleotide substrate are capable of amplification under these conditions and will generate a signal that can be coupled to the concentration of the sensor target.

C. Conformation Dependent Signaling Moieties

i. Proximity Dependent Signaling Moieties

Many proximity dependent signaling moieties are known in the art and are encompassed within the scope of the present invention (see, e.g., Morrison, 1992, in Nonisotopic DNA Probe Techniques, Kricka, ed., Academic Press, Inc., San Diego, Calif., chapter 13; and Heller and Morrison, 1985, in Rapid Detection and Identification of Infectious Agents, Academic Press, Inc., San Diego, Calif., pages 245-256), all incorporated herein by reference. Systems using these moieties rely on the change in fluorescence that occurs when the moieties are brought into close proximity. Such systems are described in the literature as fluorescence energy transfer (FET), fluorescence resonance energy transfer (FRET), nonradiative energy transfer, long-range energy transfer, dipole-coupled energy transfer, or Forster energy transfer (see, e.g., U.S. Pat. No. 5,491,063, Wu, and Brand, 1994).

Other proximity-dependent signaling systems that do not rely on direct energy transfer between signaling moieties are also known in the art and can be used in the methods described herein. These include, e.g., systems in which a signaling moiety is stimulated to fluoresce or luminesce upon activation by target molecule binding. This activation may be direct (e.g., as in the case of surface proximity assays (SPA), via a photon or radionuclide decay product emitted by the bound target), or indirect (e.g., as in the case of AlphaScreen™ assays, via reaction with singlet oxygen released from a photosensitized donor bead upon illumination). In both scenarios, the activation of detected signaling moiety is dependent on close proximity of the signaling moiety and the activating species. In general, for both fluorescence, fluorescence polarization, and scintillation-proximity-type assays, the nucleic acid sensor molecule may be utilized in either solution-phase or solid-phase formats. That is, in functional form, the nucleic acid sensor molecule may be tethered (directly, or via a linker) to a solid support or free in solution.

In one embodiment, a scintillation proximity assay (SPA) is used. In this embodiment, the nucleic acid sensor molecules are allosteric ribozymes which ligate a substrate in the presence of a target molecule (see FIGS. 2A and B) are bound to a scintillant-impregnated microwell plate (e.g., FlashPlates, NEN Life Sciences Products, Boston, Mass.) coated with, for example, streptavidin via a (biotin) linker attached to the 5′ end of the effector oligonucleotide sequence (for example, GCGACTGGACATCACGAG (SEQ ID NO:2) in FIG. 2A). The various plate-sensor coupling chemistries are determined by the type and manufacturer of the plates, and are well-known in the art. Upon the addition of a solution containing target molecule and excess radiolabeled (e.g., with ³²P or ³⁵S) substrate in ligation buffer, the ribozymes hybridize and ligate the substrate oligonucleotide. Some fraction of the radiolabeled substrate will be ligated to surface-immobilized ribozyme sensor molecules on the plate, while unligated substrate will be free in solution. Only those substrates ligated to surface-immobilized ribozyme sensor molecules on the plate will be in close enough proximity to the scintillant molecules embedded in the plate to excite them, thereby stimulating luminescence which can be easily detected using a luminometer (e.g., the TopCount luminescence plate reader, Packard Biosciences, Meriden, Conn.). This type of homogeneous assay format provides straightforward, real-time detection, quantification, and kinetic properties of target molecule binding.

In another embodiment, a similar SPA assay format is performed using scintillant-impregnated beads (e.g., Amersham Pharmacia Biotech, Inc., Piscataway, N.J.). In this embodiment, the allosteric ribozyme sensor molecules which ligate a substrate in the presence of a target molecule (see FIGS. 2A and B) are coupled to scintillant-impregnated beads which are suspended in solution in, for example, a microwell plate. The various bead-sensor coupling chemistries are determined by the type and manufacturer of the beads, and are well-known in the art. Upon the addition of a solution containing target molecule and excess radiolabeled (e.g., with ³²P or ³⁵S) substrate in ligation buffer, the ribozymes hybridize and ligate the substrate oligo. Some fraction of the radiolabeled substrate will be ligated to surface-immobilized ribozyme sensor molecules on the beads, while unligated substrate will be free in solution. Only those substrates ligated to surface-immobilized ribozyme sensor molecules on the beads will be in close enough proximity to the scintillant molecules embedded in the beads to excite them, thereby stimulating luminescence which can be easily detected using a luminometer (e.g., the TopCount luminescence plate reader, Packard Biosciences, Meriden, Conn.). In addition to enabling real-time target detection and quantification, this type of homogeneous assay format can be used to investigate cellular processes in situ in real time. This could be done by culturing cells directly onto a microwell plate and allowing uptake of scintillant beads and radioisotope by cells. Biosynthesis, proliferation, drug uptake, cell motility, etc. can then be monitored via the luminescence signal generated by beads in presence of selected target molecules (see Cook et al.,1992, or Heath et al., 1992).

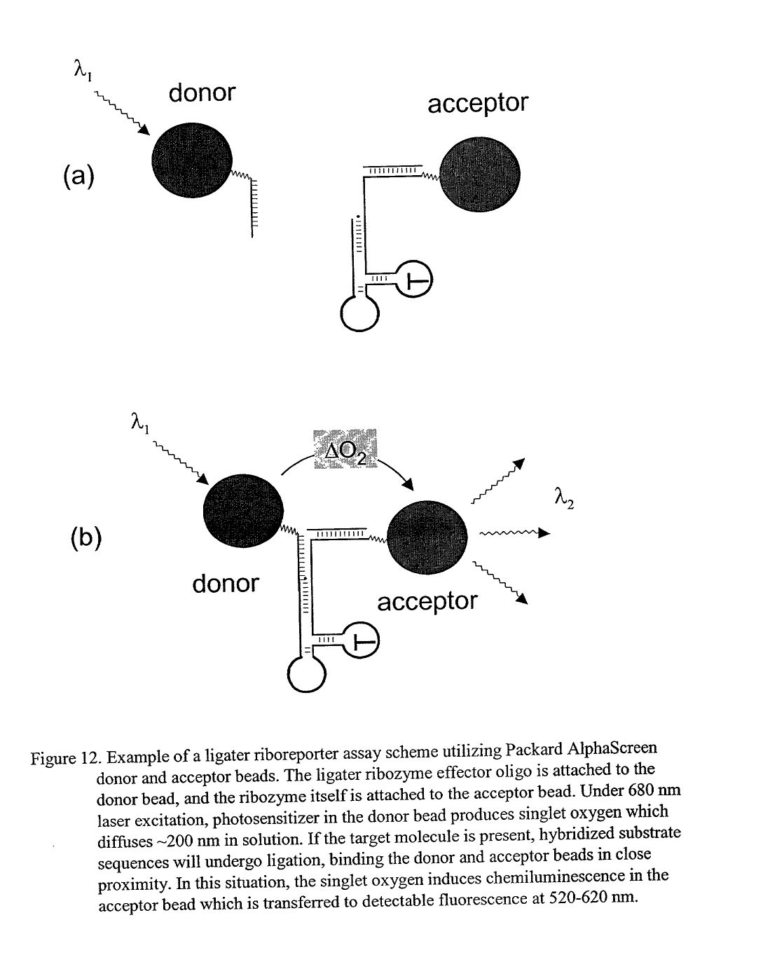

FIGS. 12A and B show an exemplary embodiment of a non-isotopic proximity assay based on nucleic acid sensor molecules used in conjunction with AlphaScreen™ beads (Packard Biosciences, Meriden, Conn.). In this embodiment, the nucleic acid sensor molecules are allosteric ribozymes which ligate a substrate in the presence of a target molecule (see FIGS. 2A and B) are bound to a chemiluminescer-impregnated acceptor bead coated with, for example, streptavidin via a (biotin) linker attached to the 5′ end of the effector oligonucleotide sequence (GCGACTGGACATCACGAG (SEQ ID NO:2) in FIG. 2A). The various bead-sensor coupling chemistries are determined by the type and manufacturer of the beads, and are well-known in the art. The substrate oligo is coupled to a photosensitizer-impregnated donor bead coated with, for example, streptavidin via a (biotin) tag attached to the 3′ end of the substrate. The donor (substrate) and acceptor (ribozyme) beads and target molecules are then combined in solution in a microwell plate, some of the ribozymes hybridize and ligate the substrate oligo, bringing the donor and acceptor beads into close proximity (<200 nm). Upon illumination at 680 nm, the photosensitizer in the donor bead converts ambient oxygen into the singlet state at a rate of approximately 60,000/second per bead. The singlet oxygen will diffuse a maximum distance of approximately 200 nm in solution; if an acceptor bead containing chemiluminescer is within this range, i.e., if ligation has occurred in the presence of the target molecule, chemiluminescence at 370 nm is generated. This radiation is immediately converted within the acceptor bead to visible luminescence at 520-620 nm with a decay half-life of 0.3 sec. The visible luminescence at 520-620 nm is detected using a time-resolved fluorescence/luminescence platereader (e.g., the Fusion multifunction plate reader, Packard Biosciences, Meriden, Conn.). This type of nonisotopic homogeneous proximity assay format provides highly sensitive, detection and quantification of target molecule concentrations in volumes <25 microliters for high throughput screening (see Beaudet et al.,2001).

Suitable fluorescent labels are known in the art and commercially available from, for example, Molecular Probes (Eugene, Oreg.). These include, e.g. donor/ acceptor (i.e., first and second signaling moieties) molecules such as: fluorescein isothiocyanate (FITC)/tetramethylrhodamine isothiocyanate (TRITC), FITC/Texas Red TM Molecular Probes), FITC/N-hydroxysuccinimidyl 1-pyrenebutyrate (PYB), FITC/eosin isothiocyanate (EITC), N-hydroxysuccinimide 1-pyrenesulfonate (PYS)/FITC, FITC/Rhodamine X (ROX), FITC/tetramethylrhodamine (TAMRA), and others. In addition to the organic fluorophores already mentioned, various types of nonorganic fluorescent labels are known in the art and are commercially available from, for example, Quantum Dot Corporation, Inc., Hayward Calif.). These include, e.g., donor/ acceptor (i.e., first and second signaling moieties) semiconductor nanocrystals (i.e., ‘quantum dots’) whose absorption and emission spectra can be precisely controlled through the selection of nanoparticle material, size, and composition (see, for example, Bruchez et al., 1998, Chan and Nie, 1998, Han et al., 2001).

The selection of a particular donor/acceptor pair is not critical to practicing the invention provided that energy can be transferred between the donor and the acceptor. P-(dimethyl aminophenylazo) benzoic acid (DABCYL) is one example of a non-fluorescent acceptor dye which effectively quenches fluorescence from an adjacent fluorophore, e.g., fluorescein or 5-(2′-aminoethyl) aminonaphthalene (EDANS).

FIGS. 3A and B and 6A and B show exemplary nucleic acid sensor molecules derived from nucleic acid precursor molecules (FIGS. 2A and B and 5A and B, respectively), according to embodiments. FIG. 3 shows a nucleic acid sensor molecule obtained from an oligonucleotide pool in which the catalytic site was a ligase site. FIG. 6 shows a nucleic acid sensor molecule obtained from an oligonucleotide pool in which the catalytic site was a site mediating self-cleavage. In the embodiment shown in FIGS. 3A and B, a nucleic acid sensor precursor molecule from which a portion of a ligase site (e.g., the AGUCG sequence at the 3′ end of the nucleic acid sensor precursor molecule, as shown in FIG. 2) has been removed is coupled to a first signaling moiety (F) at a first nucleotide (1) and to a second signaling moiety (D) at a second nucleotide (2). In a further embodiment, the first and second signaling moieties molecules are attached to non-terminal sequences. The position of the non-terminal sequences coupled to signaling moieties is limited to a maximal distance from the 5′ or 3′ nucleotide which still permits proximity dependent changes in the optical properties of the molecule. Coupling chemistries are routinely practiced in the art, and oligonucleotide synthesis services provided commercially (e.g., Integrated DNA Technologies, Coralville, Iowa) can also be used to generate labeled molecules. In a further embodiment, the nucleic acid sensor molecule is used, either tethered to a solid support or free in solution, to detect the presence and concentration of target molecules in a complex biological fluid.

In the embodiment shown in FIGS. 3A and B, the first signaling moiety (F) is a fluorescein molecule coupled to the 5′ end and the second signaling molecule (D) is a DABCYL molecule (a quenching group) coupled to the 3′ end. Because of the nearly complete base pairing of the non-target molecule bound form (see FIG. 3B), this is the favored form of the nucleic acid sensor molecule in the absence of the target molecule. When the nucleic acid sensor molecule is not bound by target molecule, the fluorescent group and the quenching group are in close proximity and little fluorescence is detectable from the fluorescent group. Addition of target molecule causes a change in the conformation of the nucleic acid sensor molecule shown in FIG. 3B to that shown in FIG. 3A. When the molecule assumes the conformation shown in FIG. 3A, the first and second signaling moieties (F and D, respectively) are no longer in sufficient proximity for the quenching group to quench the fluorescence of the fluorescent group, resulting in a detectable fluorescent signal being produced upon binding of the target molecule.

In one embodiment, the ligand-binding domain sequence from a previously identified nucleic acid sensor molecule is incorporated into a separate oligonucleotide sequence which changes conformation upon target binding, as shown in FIGS. 91A and B, and FIGS. 6A and B. During or after synthesis, an optical signal generating unit is either added or inserted into the oligonucleotide sequence comprising the derived nucleic acid sensor molecule. As noted previously, nucleic acid sensor molecules of this type can be derived from allosteric ribozymes, such as those derived from the hammerhead, hairpin, L1 ligase, or group 1 intron ribozymes and the like, or may be derived from aptamer beacons or signaling aptamers (see Soukup et al., 2001, or Hamaguchi et al., 2001), all of which transduce molecular recognition into a detectable optical signal.

In the embodiment shown in FIGS. 9A and B, the nucleic acid sensor molecules are comprised of single stranded DNA, with the following sequence: 5′-CCAACGGTTGGTGTGGTTGG-3′ (SEQ ID NO:8). The first signaling moiety is a fluorescein-based label (FAM) attached to the 5′ terminus, and the second signaling moiety is a quenching group (DABCYL) attached to the 3′ end. The final configuration of the nucleic acid sensor molecule is: 5′-FAM-CCAACGGTTGGTGTGGTTGG-DABCYL-3′. In the unbound state, the nucleic acid sensor molecule forms a duplex stem-loop. In the presence of the target (here thrombin), the sensor molecule assumes the G-quadruplex structure. This conformational rearrangement results in a change in the distance between the 5′-attached fluorophore end and the 3′-attached quencher. With the quencher separated from the fluorophore by a distance greater than the Forster radius (radius for 50% resonance energy transfer, typically ˜10-50 Angstroms), the detected fluorescence emission intensity from the fluorophore increases sharply, as shown in FIG. 9C. At saturating concentrations of target molecule, the increase in fluorescence intensity from the sensor molecule was approximately 2.5-fold (see Hamaguchi, et al, 2001).

In the embodiment shown in FIGS. 10A and B, a fluorescently labeled self-cleaving ribozyme such as the hammerhead (in this case attached to a solid support via a linker molecule) in the unbound state is hybridized with a transacting substrate which bears a second fluorescent label. In the unbound state, i.e., in the absence of target, the donor fluorophore (on the ribozyme) and the acceptor fluorophore (on the substrate) are in sufficiently close proximity for FRET to occur; thus, minimal fluorescent emission is detected from the donor fluorophore at wavelength 2, λ2, upon epi-illumnation excitation at wavelength 1, λ1. Upon target binding, the cleavage fragment of the substrate bearing the acceptor fluorophore dissociates from the ribozyme-target complex. Once separated from the acceptor fluorophore, the donor fluorophore can no longer undergo de-excitation via FRET, resulting in a detectable increase in its fluorescent emission at wavelengthc2, λ2 (see, for example, Singh. et al., 1999; Wu, and Brand, 1994; Walter and Burke, 1997; Walter et al., 1998). In a further embodiment, the change in the polarization state of the fluorescent emission from the donor fluorophore (due to the increased diffusional rotation rate of the smaller cleavage fragment) can be detected/monitored in addition to changes in fluorescent emission intensity (see, for Singh, 2000). In a further embodiment, the ribozyme sensor molecules are free in solution.

In another embodiment, shown in FIG. 10B, the acceptor fluorophore attached to the substrate is replaced by a quencher group. This replacement will also result in minimal fluorescent donor emission at wavelength 2, λ2, with the ribozyme is in the unbound state under epi-illumination excitation at wavelength 1, λ1. Upon target binding, the cleavage fragments of the substrate bearing the donor and quencher groups dissociate from the ribozyme-target complex. Once separated from the quencher, the donor fluorophore will exhibit a detectable increase in its fluorescent emission at wavelength 2, λ2. In a further embodiment, the change in the polarization state of the fluorescent emission from the donor fluorophore (due to the increased diffusional rotation rate of the smaller cleavage fragment) can be detected/monitored in addition to changes in fluorescent emission intensity. In a further embodiment, the ribozyme sensor molecules are free in solution.

In a different embodiment, the optical configuration is designed to provide excitation via total internal reflection (TIR)-illumination, as shown in FIG. 10C. Also, the donor fluorophore is attached to the ribozyme body while the acceptor is attached to the substrate. In this configuration, with the surface-immobilized ribozyme in the unbound state, the fluorescent donor emission at wavelength 2, λ2, will be minimal. Upon target binding, the cleavage fragment of the substrate bearing the quencher group dissociates from the ribozyme-target complex. Once separated from the quencher, the donor fluorophore will exhibit a detectable increase in its fluorescent emission at wavelength 3, λ3. In an alternative embodiment to that shown in shown in FIG. 10C, the quencher group can be replaced with an acceptor fluorophore. In yet another alternative embodiment to those shown in FIGS. 10A, B, and C, the donor fluorophore is coupled to the cleavage fragment of the ribozyme substrate and the acceptor fluorophore or quencher group is deleted. Upon target binding and dissociation of the cleavage fragment, the polarization state of the fluorescent emission from the donor fluorophore will undergo a detectable change due to the difference in the diffusional rotation rates of the surface-bound ribozyme-target complex and the free cleavage fragment.

In one embodiment, a universal FRET trans-substrate is synthesized for all sensor molecules derived from self-cleaving allosteric ribozymes. This substrate would have complementary optical signaling units (i.e., donor and acceptor groups) coupled to opposite ends of the synthetic oligonucleotide sequence. Such a universal substrate would obviate the need for coupling optical signaling units to the sensor (i.e., ribozyme) molecule itself.

The relative stabilities of the bound and unbound forms of the nucleic acid sensor molecules is optimized to achieve the highest sensitivity of detection of target molecule. In one embodiment, the nucleic acid sensor molecule is further engineered to enhance the stability of one form over another. In one embodiment, the UA marked in bold in FIGS. 3A and B is changed to a CC, favoring the formation of the target molecule bound form. Because these bases do not form base pairs when the nucleic acid sensor molecule is unbound, the unbound form is not stabilized.

In addition to the herein described methods, any additional proximity dependent signaling system known in the art can be used to practice the method according to the invention, and are encompassed within the scope.

A number of methods can be used to evaluate the relative stability of different conformations of the nucleic acid sensor molecule. In one embodiment, the free energy of the structures formed by the nucleic acid sensor molecule is determined using software programs such as mfold®, which can be found on the Rensselaer Polytechnic Institute (RPI) web site (www.rpi.edu/dept.).

In another embodiment, a gel assay is performed which permits detection of different conformations of the nucleic acid sensor molecule. In this embodiment, the nucleic acid sensor molecule is allowed to come to equilibrium at room temperature or the temperature at which the nucleic acid sensor molecule will be used. The molecule is then cooled to 4° C. and electrophoresed on a native (non-denaturing) gel at 4° C. Each of the conformations formed by the nucleic acid sensor molecule will run at a different position on the gel, allowing visualization of the relative concentration of each conformation. Similarly, the conformation of nucleic acid sensor molecules which forms in the presence of target molecule is then determined by a method such as circular dichroism (CD). By comparing the conformation of the nucleic acid sensor molecule formed in the presence of target molecule with the conformations formed in the absence of target molecule, the conformation which corresponds to the bound conformation can be identified in a sample in which there is no target molecule. The nucleic acid sensor molecule can then be engineered to minimize the formation of the bound conformation in the absence of target molecule. The sensitivity and specificity of nucleic acid sensor molecule can be further assayed for using target molecule binding assays with known amounts of target molecules.

In another embodiment, shown in FIGS. 6A and B, a nucleic acid sensor precursor molecule from which a portion of a self-cleaving site has been removed, is coupled to a first signaling moiety (F) at a first nucleotide and to a second signaling moiety (D) at a second nucleotide. In this embodiment, the entire catalytic site of the nucleic acid precursor molecule (see FIGS. 5A and B) has been removed. In one embodiment (FIGS. 6A and B), additional bases (e.g., UGGUAU) are added to one end of the portion of the nucleic acid sensor molecule comprising the target activation site sequence, to stabilize the unbound form of the nucleic acid sensor molecule (FIG. 6B). These bases are selected to be complementary to bases at the opposite end of the nucleic acid sensor molecule (ACCAUA). Additional bases may be added to either the 5′ or the 3′ end of the nucleic acid sensor molecule.

Modifications to stabilize one conformation of the nucleic sensor molecule over another may be identified using the mfold program or native gel assays discussed above. A labeled nucleic acid sensor molecule is generated by coupling a first signaling moiety (F) to a first nucleotide and a second signaling moiety (D) to first and second nucleotides as discussed above. As above, the sensitivity and specificity of the nucleic acid sensor molecule can be further assayed for using target molecule binding assays with known amounts of target molecules. In further embodiments , nucleic acid sensor molecules are selected which have optimal affinity for a target molecule using an affinity fingerprinting technique as disclosed in U.S. Pat. No. 5,587,293, the entirety of which is incorporated by reference herein.

ii. Optical Signal Generating Units with Single Signaling Moieties

In one embodiment, the nucleic acid sensor molecule comprises an optical signaling unit with a single signaling moiety introduced at either an internal or terminal position within the nucleic acid sensor molecule. In this embodiment, binding of the target molecule results in changes in both the conformation and physical aspect (e.g., molecular volume or mass, rotational diffusion rate, etc.) of the nucleic acid sensor molecule. Conformational changes in the nucleic acid sensor molecule upon target binding will modify the chemical environment of the signaling moiety. Such a change in chemical environment will in general change the optical properties of the signaling moiety. Suitable signaling moieties are described in Jhaveri, et al, 2000, and include, e.g. fluorescein, acridine, and other organic and nonorganic fluorophores.

In one embodiment, a signaling moiety is introduced at a position in the nucleic acid precursor molecule near the target activation site (identifiable by footprinting studies, for example). Binding of the target molecule will (via a change in conformation of the nucleic acid precursor molecule) alter the chemical environment and thus affect the optical properties of the signaling moiety in a detectable manner.

Binding of the nucleic acid sensor molecule with the target molecule will result in changes in the conformation and physical aspect of the nucleic acid sensor molecule, and will thus alter the kinetic properties of the signaling moiety. In particular, the changes in conformation and mass of the sensor-target complex will reduce the rotational diffusion rate for the sensor-target complex, resulting in a detectable change in the observed steady state fluorescence polarization (FP) from the signaling moiety. The expected change in FP signal with target concentration can be derived using a modified form of the well-known Michaelis-Menten model for ligand binding kinetics (ref: Lakowicz, 1999). FP is therefore a highly sensitive means of detecting and quantitatively determining the concentration of target molecules in a sample solution (Jameson and sawyer, 1995; Jameson and Seifried, 1999; Jolley, 1999; Singh, 2000; Owicki et al., 1997). FP methods are capable of functioning in both solution- and solid-phase implementations.

Numerous additional methods can be used that, e.g., make use of a single fluorescent label and an unpaired guanosine residue (instead of a quencher group), to enable the use of FRET in target detection and quantitation as described in the embodiments previous section (see Walter and Burke, 1997).

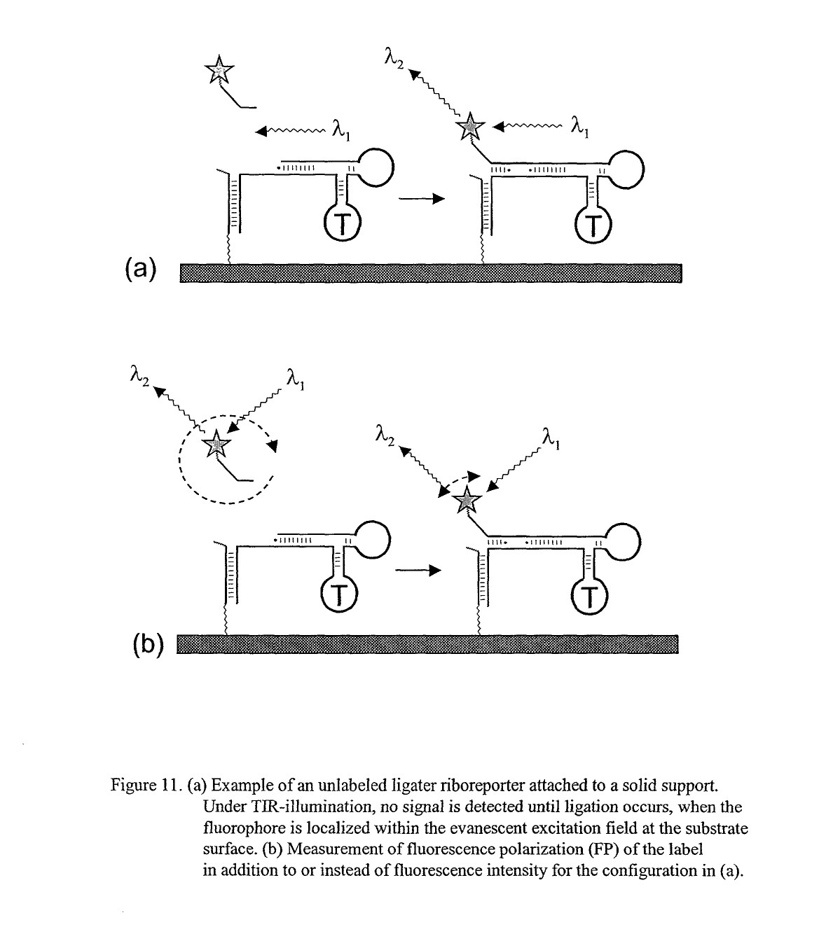

In a further embodiment, shown in FIGS. 11A and B, the labeled self-cleaving ribozyme is replaced by an unlabeled ligating ribozyme such as the lysozyme-dependent L1 ligase (see, for example, Robertson, M. P. and Ellington, A. D, 2000). In the unbound state, i.e., in the absence of target, no fluorescent emission is detected from the surface-bound ribozymes under total internal reflection (TIR)-illumination. Upon binding of target molecules in the presence of a substrate with a tag (where the tag is capable of binding to a subsequently added fluorescent label via interactions including, but not limited to, biotin/steptavidin, amine/aldehyde, hydrazide, thiol, or other reactive groups) those substrates hybridized to ribozymes will undergo ligation and become covalently bonded to the ribosome. In order to maximize the probability of hybridization for a given ribozyme, substrate can be added in excess relative to ribozyme, the temperature of the ambient solution in which the reaction takes place can be kept below room temperature (e.g., 4 degrees C.), and agitation of the reaction vessel can be employed to overcome the kinetic limitation of diffusion-limited transport of species in solution. Given the above conditions, as well as sufficient time for maximal hybridization and subsequent ligation to occur, fluorescent label with the appropriate reactive group to bind the substrate tag is added to the reaction mixture. Again, the degree of substrate-label binding can be maximized through control of label concentration, solution temperature, and agitation. Once the fluorescent label has bound to all available ligated substrate-ribozyme-target complex, the solution temperature can be raised to drive off all of the hybridized but unligated substrate. With TIR-illumination, the spatial extent of the excitation region above the solid substrate surface to which the ribozymes are bound is only on the order of 100 nm. Therefore, the bulk solution above the substrate surface is not illuminated and the detected fluorescent emission will be primarily due to fluorophores which are bound to ligated substrate-ribozyme-target complexes tethered to the substrate surface. The fluorescence emission from surface-bound ribozyme-target complexes in this homogeneous solid phase assay format represents an easily detectable optical signal. In another embodiment, the fluorescence polarization (FP) of the labeled substrate can be monitored. Upon ligation, the steady state fluorescence polarization signal form the substrate-ribozyme complex will increase detectably relative to the FP signal from the free labeled substrate in solution, due to the difference in the diffusional rotation rates between the free and ligated forms.

In another embodiment, an unlabeled ligating ribozyme such as the lysozyme-dependent L1 ligase (see, for example, Robertson, M. P. and Ellington, A. D, 2000) is bound to a solid surface. In this embodiment, the substrate oligo is coupled to an enzyme-linked luminescent moiety, such as horse radish peroxidase (HRP) by a tag (where the tag is capable of binding to a subsequently added label via interactions including, but not limited to, biotin/steptavidin, amine/aldehyde, hydrazide, thiol, or other reactive groups). In the unbound state, i.e., in the absence of target, no luminescent emission is detected from the surface-bound ribozymes. Upon binding of target molecules in the presence of labeled substrate, those substrates hybridized to ribozymes will undergo ligation and become covalently bonded to the ribosome. After removal of excess, unbound ribozyme substrate, the activation substrate for the enzyme-linked luminescent label is added to the reaction volume. The resulting luminescent signal (e.g., from HRP, luciferase, etc.) is easily detectable using standard luminometers (e.g., the Fusion multifunction plate reader, Packard Bioscience). In a further embodiment, the activated solution can be precipitated, followed by colorimetric detection.

2. Generating Target Activated Biosensors

Target activated biosensors for the detection of a target molecule of interest are generated by first selecting nucleic acid precursor molecules with catalytic activity modifiable (e.g., activatable) by a selected target molecule. In one embodiment, at least a portion of the catalytic site of the precursor molecule is then removed and an optical signal generating unit is either added or inserted. Binding of the target molecule to the target activated biosensor activates a change in the properties of the optical signaling unit.

In one embodiment, a target activated biosensor is provided which comprises a plurality of nucleic acid sensor molecules labeled with first and second signaling moieties specific for a target molecule. In another embodiment, sensor molecules are labeled with a single signaling moiety. In one embodiment, the labeled nucleic acid sensor molecules are provided in a solution (e.g., a buffer). In another embodiment, the labeled nucleic acid sensor molecules are attached directly or indirectly (e.g., through a linker molecule) to a substrate. In further embodiments, nucleic acid sensor molecules can be synthesized directly onto the substrate. Suitable substrates which are encompassed within the scope include, e.g., glass or quartz, silicon, encapsulated or unencapsulated semiconductor nanocrystal materials (e.g., CdSe), nitrocellulose, nylon, plastic, and other polymers. Substrates may assume a variety of configurations (e.g., planar, slide shaped, wafers, chips, tubular, disc-like, beads, containers, or plates, such as microtiter plates, and other shapes).

Numerous attachment chemistries, both direct and indirect, can be used to immobilize the sensor molecules on a solid support. These include, e.g., amine/aldehyde, biotin/streptavidin (avidin, neutravidin), ADH/oxidized 3′ RNA. In a particular embodiment, the nucleic acid sensor molecules are allosteric ribozymes which ligate a substrate in the presence of a target molecule (see FIGS. 2A and B). In this embodiment the ribozymes are bound to a solid substrate via the effector oligonucleotide sequence (for example, GCGACTGGACATCACGAG (SEQ ID NO:2) in FIG. 2A).

In one embodiment, a manual or computer-controlled robotic microarrayer is used to generate arrays of nucleic acid sensor molecules immobilized on a solid substrate. In one embodiment, the arrayer utilizes contact-printing technology (i.e., employing printing pins of metal, glass, etc., with or without quill-slots or other modifications). In a different embodiment, the arrayer utilizes non-contact printing technology (i.e., employing ink jet or capillary-based technologies, or other means of dispensing a solution containing the material to be arrayed). Numerous methods for preparing, processing, and analyzing microarrays are known in the art (see Schena et al., 2000; Mace et al., 2000; Heller et al., 1999; Basararsky et al., 2000; Schermer, 1999). Robotic and manual arrayers are commercially available, for example, the SpotArray from Packard Biosciences, Meriden, Conn., and the RA-1 from GenomicSolutions, Ann Arbor, Minn.).

In one embodiment, larger substrates can be generated by combining a plurality of smaller biosensors forming an array of biosensors. In a further embodiment, nucleic acid sensor molecules placed on the substrate are addressed (e.g., by specific linker or effector oligonucleotide sequences on the nucleic acid sensor molecule) and information relating to the location of each nucleic acid sensor molecule and its target molecule specificity is stored within a processor. This technique is known as spatial addressing or spatial multiplexing. Techniques for addressing nucleic acids on substrates are known in the art and are described in, for example, U.S. Pat. Nos. 6,060,252, 6,051,380, 5,763,263, 5,763,175, 5,741,462, the entireties of which are incorporated by reference herein.

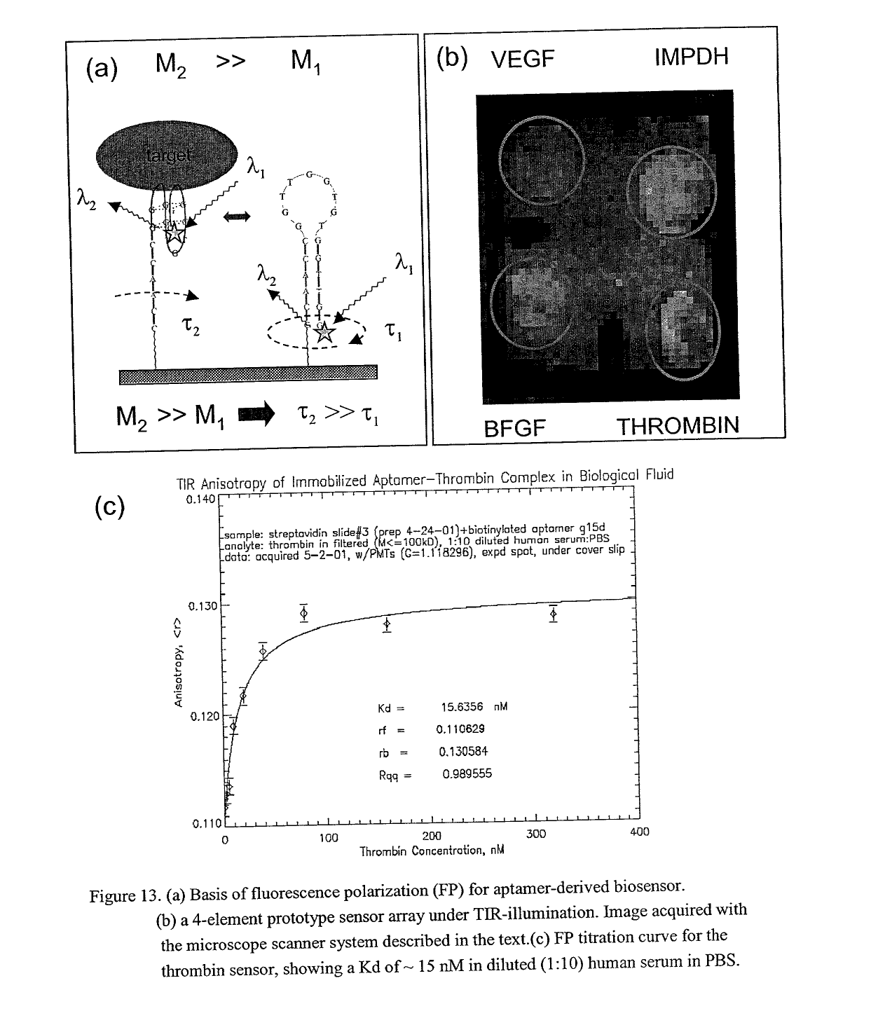

In the embodiment shown in FIGS. 13A and b, 4 different nucleic acid sensor molecules are immobilized on a streptavidin-derivatized glass substrate via biotin linkers. The biosensor targets for the sensors are 4 different physiological proteins: thrombin, IMPDH, VEGF, and BFGF. Each biosensor is labeled with a single 5′ FAM (fluorescein) group. The individual sensor spots in this case were manually arrayed. Solution measurements of target concentration are made by bathing the surface of the biosensor array in a solution containing the targets (analytes) of interest. In practice this in accomplished either by incorporating the array within a microflowcell (with a flow rate of ˜25 microliters/min), or by placing a small volume (˜6-10 microliters) of the target solution on the array surface and covering it with a cover slip. Detection and quantification of target (here protein) concentration is accomplished by monitoring changes in the fluorescence polarization (FP) signal emitted from the fluorescein label under illumination by 488 nm laser radiation. FIG. 13A shows the physical basis for the change in the polarization state of the emitted fluorescence from the biosensors. The rotational diffusion rate is inversely proportional to the molecular volume; thus the rotational correlation time for the roughly 20-nucleotide unbound sensor (i.e., in the absence of target) will be significantly less than that for the >55 kD target-sensor complex. The fluorescence emission from the target-sensor complex will therefore experience greater residual polarization due to the smaller angle through which the emission dipole axis of the sensor fluorophore can rotate within its radiative lifetime. The polarization of the detected fluorescence emission at approximately 516 nm quantitatively correlated with protein concentration, as shown in FIG. 13C. Titration of protein into the interrogated volume allows the determination of the dissociation constant, K_{d}, for the target-sensor interaction via fitting to a Michaelis-Menten model for ligand-binding kinetics (ref: Lakowicz, 1999). Such an FP titration curve is shown in FIG. 13C for the thrombin-specific biosensor in biological fluid consisting of 10% human serum in phosphate buffered saline (PBS). In another embodiment, different surface attachment chemistries are used to immobilize the biosensors on a solid substrate. As previously noted, these include, e.g., interactions involving biotin/steptavidin, amine/aldehyde, hydrazide, thiol, or other reactive groups.

The specificity of the target activated biosensor according to the invention is determined by the specificity of the target activation site of the nucleic acid sensor molecule. In one embodiment, a nucleic acid biosensor is provided in which all of the nucleic acid sensor molecules recognize the same molecule. In another embodiment, a nucleic acid biosensor is provided which can recognize at least two different target molecules allowing for multi-analyte detection. Multiple analytes can be distinguished by using different combinations of first and second signaling molecules. In addition to the wavelength/color and spatial multiplexing techniques previously described, nucleic acid biosensors may be used to detect multiple analytes using intensity multiplexing. This is accomplished by varying the number of fluorescent label molecules on each biosensor molecule in a controlled fashion. Since a single fluorescent label is the smallest integral labeling unit possible, the number of fluorophores (i.e., the intensity from) a given biosensor molecule provides a multiplexing index. Using the combination of 6-wavelength (color) and 10-level intensity multiplexing, implemented in the context of semiconductor nanocrystals derivatized as bioconjugates, would theoretically allow the encoding of million different analyte-specific biosensors (Han et al., 2001).

In one embodiment, multiple single target biosensors can be combined to form a multianalyte detection system which is either solution-based or substrate-based according to the needs of the user. In this embodiment, individual biosensors can be later removed from the system, if the user desires to return to a single analyte detection system (e.g., using target molecules bound to supports, or, for example, manually removing a selected biosensor(s) in the case of substrate-based biosensors). In a further embodiment, nucleic acid sensor molecules binding to multiple analytes are distinguished from each other by referring to the address of the nucleic acid sensor molecule on a substrate and correlating its location with the appropriate target molecule to which it binds (previously described as spatial addressing or multiplexing).

In one embodiment, subsections of a biosensor array can be individually subjected to separate analyte solutions by use of substrate partitions or enclosures that prevent fluid flow between subarrays, and microfluidic pathways and injectors to introduce the different analyte solutions to the appropriate sensor subarray.

3. Target Activated Biosensor Systems

In one embodiment, a target activated biosensor system is provided comprising a target activated biosensor in communication with a detector system. In a further embodiment, a processor is provided to process optical signals detected by the detector system. In still a further embodiment, the processor is connectable to a server which is also connectable to other processors. In this embodiment, optical data obtained at a site where the biosensor system resides can be transmitted through the server and data is obtained, and a report displayed on the display of the off-site processor within seconds of the transmission of the optical data. In one embodiment, data from patients is stored 10 in a database which can be accessed by a user of the system.

Data obtainable from the biosensors according to the invention include diagnostic data, data relating to lead compound development, and nucleic acid sensor molecule modeling data (e.g., information correlating the sequence of individual sensor molecules with binding affinity for a particular target molecule). In one embodiment, these data are stored in a computer database. In a further embodiment, the database includes, along with diagnostic data obtained from a sample by the biosensor, information relating to a particular patient, such as medical history and billing information. Although, in one embodiment, the database is part of the target activated biosensor system, the database can be used separately with other detection assay methods and drug development methods.

Detectors used with the target activated biosensor systems according to the invention, can vary, and include any suitable detectors for detecting optical changes in nucleic acid molecules. These include, e.g., photomultiplier tubes (PMTs), charge coupled devices (CCDs), intensified CCDs, and avalanche photodiodes (APDs). In one embodiment, a target activated biosensor comprising labeled nucleic acid sensor molecules is excited by a light source in communication with the biosensor. In a further embodiment, when the optical signaling unit comprises first and second signal moieties that are donor/acceptor pairs (i.e., signal generation relies on the fluorescence of a donor molecule when it is removed from the proximity of a quencher acceptor molecule), recognition of a target molecule will cause a large increase in fluorescence emission intensity over a low background signal level. The high signal-to-noise ratio permits small signals to be measured using high-gain detectors, such as PMTs or APDs. Using intensified CCDs, and PMTs, single molecule fluorescence measurements have been made by monitoring the fluorescence emission, and changes in fluorescence lifetime, from donor/acceptor FRET pairs (see Sako, et al., 2000; Lakowicz et al, 1991)).

Light sources include, e.g., filtered, wide-spectrum light sources, (e.g., tungsten, or xenon arc), laser light sources, such as gas lasers, solid state crystal lasers, semiconductor diode lasers (including multiple quantum well, distributed feedback, and vertical cavity surface emitting lasers (VCSELs)), dye lasers, metallic vapor lasers, free electron lasers, and lasers using any other substance as a gain medium. Common gas lasers include Argon-ion, Krypton-ion, and mixed gas (e.g., Ar—Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 nm. Typical output lines from solid state crystal lasers include 532 nm (doubled Nd:YAG) and 408/816 nm (doubled/primary from Ti:Sapphire). Typical output lines from semiconductor diode lasers are 635, 650, 670, and 780 nm.

Excitation wavelengths and emission detection wavelengths will vary depending on the signaling moieties used. In one embodiment, where the first and second signaling moieties are fluorescein and DABCYL, the excitation wavelength is 488 nm and the emission wavelength is 514 nm. In the case of semiconductor nanocrystal-based fluorescent labels, a single excitation wavelength or broadband UV source may be used to excite several probes with widely spectrally separated emission wavelengths (see Bruchez et al., 1998; Chan et al., 1998).

In one embodiment, detection of changes in the optical properties of the nucleic acid sensor molecules is performed using any of a cooled CCD camera, a cooled intensified CCD camera, a single-photon-counting detector (e.g., PMT or APD), or other light sensitive sensor. In one embodiment, the detector is optically coupled to the target activated biosensor through a lens system, such as in an optical microscope (e.g., a confocal microscope). In another embodiment, a fiber optic coupler is used, where the input to the optical fiber is placed in close proximity to the substrate surface of a biosensor, either above or below the substrate. In yet another embodiment, the optical fiber provides the substrate for the attachment of nucleic acid sensor molecules and the biosensor is an integral part of the optical fiber.

In one embodiment, the interior surface of a glass or plastic capillary tube provides the substrate for the attachment of nucleic acid sensor molecules. The capillary can be either circular or rectangular in cross-section, and of any dimension. The capillary section containing the biosensors can be integrated into a microfluidic liquid-handling system which can inject different wash, buffer, and analyte-containing solutions through the sensor tube. Spatial encoding of the sensors can be accomplished by patterning them longitudinally along the axis of the tube, as well as radially, around the circumference of the tube interior. Excitation can be accomplished by coupling a laser source (e.g., using a shaped output beam, such as from a VCSEL) into the glass or plastic layer forming the capillary tube. The coupled excitation light will undergo TIR at the interior surface/solution interface of the tube, thus selectively exciting fluorescently labeled biosensors attached to the tube walls, but not the bulk solution. In one embodiment, detection can be accomplished using a lens-coupled or proximity-coupled large area segmented (pixilated) detector, such as a CCD. In a particular embodiment, a scanning (i.e., longitudinal/axial and azimuthal) microscope objective lens/emission filter combination is used to image the biosensor substrate onto a CCD detector. In a different embodiment, a high resolution CCD detector with an emission filter in front of it is placed in extremely close proximity to the capillary to allow direct imaging of the biosensors. In a different embodiment, highly efficient detection is accomplished using a mirrored tubular cavity that is elliptical in cross-section. The sensor tube is placed along one focal axis of the cavity, while a side-window PMT is placed along the other focal axis with an emission filter in front of it. Any light emitted from the biosensor tube in any direction will be collected by the cavity and focused onto the window of the PMT.

In still another embodiment, the optical properties of a target activated biosensor are analyzed using a spectrometer (e.g., such as a luminescence spectrometer) which is in communication with the biosensor. The spectrometer can perform wavelength discrimination for excitation and detection using either monochromators (i.e., diffraction gratings), or wavelength bandpass filters. In this embodiment, biosensor molecules are excited at absorption maximum appropriate to the signal labeling moieties being used (e.g., acridine at 450 nm, fluorescein at 495 nm) and fluorescence intensity is measured at emission wavelengths appropriate for the labeling moiety used (e.g., acridine at 495 nm; fluorescein at 515 nm). Achieving sufficient spectral separation (i.e., a large enough Stokes shift) between the excitation wavelength and the emission wavelength is critical to the ultimate limit of detection sensitivity. Given that the intensity of the excitation light is much greater than that of the emitted fluorescence, even a small fraction of the excitation light being detected or amplified by the detection system will obscure a weak biosensor fluorescence emission signal. In one embodiment, the biosensor molecules are in solution and are pipetted (either manually or robotically) into a cuvette or a well in a microtiter plate within the spectrometer. In a further embodiment, the spectrometer is a multifunction plate reader capable of detecting optical changes in fluorescence or luminescence intensity (at one or more wavelengths), time-resolved fluorescence, fluorescence polarization (FP), absorbance (epi and transmitted), etc., such as the Fusion multifunction plate reader system (Packard Biosciences, Meriden, Conn.). Such a system can be used to detect optical changes in biosensors either in solution, bound to the surface of microwells in plates, or immobilized on the surface of solid substrate (e.g., a biosensor microarray on a glass substrate). This type of multiplate/multisubstrate detection system, coupled with robotic liquid handling and sample manipulation, is particularly amenable to high-throughput, low-volume assay formats.

In embodiments where nucleic acid sensor molecules are attached to substrates, such as a glass slide or in microarray format, it is desirable to reject any stray or background light in order to permit the detection of very low intensity fluorescence signals. In one embodiment, a small sample volume (˜10 nL) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

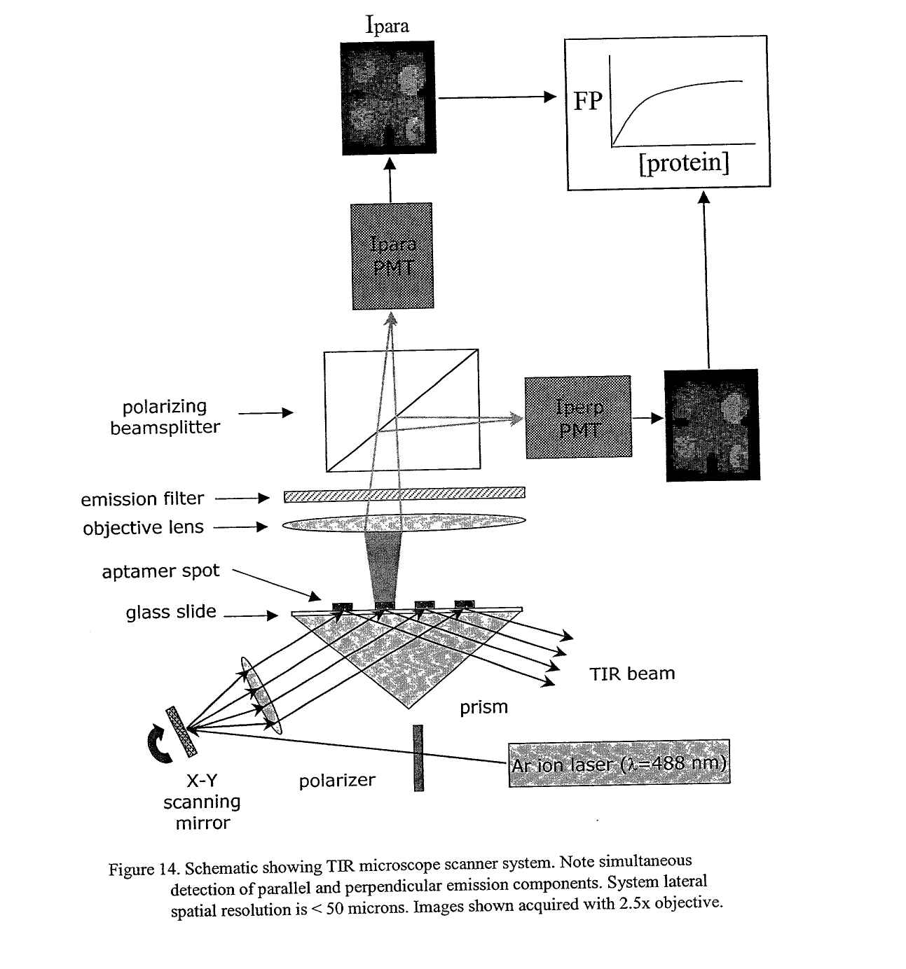

In one embodiment, spatial discrimination of nucleic acid sensor molecules attached to a substrate in a direction normal to the interface of the substrate (i.e., excitation of only a small thickness of the solution layer directly above and surrounding the plane of attachment of the biosensor molecules to the substrate surface) is obtained by evanescent wave excitation. This is illustrated in FIG. 14. Evanescent wave excitation utilizes electromagnetic energy that propagates into the lower-index of refraction medium when an electromagnetic wave is totally internally reflected at the interface between higher and lower-refractive index materials. In this embodiment a collimated laser beam is incident on the substrate/solution interface (at which the biosensors are immobilized) at an angle greater than the critical angle for total internal reflection (TIR). This can be accomplished by directing light into a suitably shaped prism or an optical fiber. In the case of a prism, as shown in FIG. 14, the substrate is optically coupled (via index-matching fluid) to the upper surface of the prism, such that TIR occurs at the substrate/solution interface on which the biosensors are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the nucleic acid sensor, whether a change in intensity or polarization. Spatial discrimination in the plane of the interface (i.e., laterally) is achieved by the optical system.

In one embodiment, a large area of the biosensor substrate is uniformly illuminated, either via evanescent wave excitation or epi-illumination from above, and the detected signal is spatially encoded through the use of a pixelated detector, such as CCD camera. An example of this type of uniform illumination/CCD detection system (using epi-illumination) ) for the case of microarrayed biosensors on solid substrates is the GeneTAC 2000 scanner (GenomicSolutions, Ann Arbor, Mich.). In a different embodiment, a small area (e.g., 10×10 microns to 100×100 microns) of the biosensor substrate is illuminated by a micro-collimated beam or focused spot. In one embodiment, the excitation spot is rastered in a 2-dimensional scan across the static biosensor substrate surface and the signal detected (with an integrating detector, such as a PMT) at each point correlated with the spatial location of that point on the biosensor substrate (e.g., by the mechanical positioning system responsible for scanning the excitation spot). Two examples of this type of moving spot detection system for the case of microarrayed biosensors on solid substrates are: the DNAScope scanner (confocal, epi-illumination, GeneFocus, Waterloo, ON, Canada), and the LS IV scanner (non-confocal, epi-illumination, GenomicSolutions, Ann Arbor, Mich.). In yet another embodiment, a small area (e.g., 10×10 microns to 100×100 microns) of the biosensor substrate is illuminated by a stationary micro-collimated beam or focused spot, and the biosensor substrate is rastered in a 2-dimensional scan beneath the static excitation spot, with the signal detected (with an integrating detector, such as a PMT) at each point correlated with the spatial location of that point on the biosensor substrate (e.g., by the mechanical positioning system responsible for scanning the substrate). An example of this type of moving substrate detection (using confocal epi-illumination) system for the case of microarrayed biosensors on solid substrates is the ScanArray 5000 scanner (Packard Biochip, Billerica, Mass.).

In the embodiment shown in FIG. 14, a TIR evanescent wave excitation optical configuration is implemented, with a static substrate and dual-capability detection system. The detection system is built on the frame of a Zeiss universal fluorescence microscope. The system is equipped with 2 PMTs on one optical port, and an intensified CCD camera (Cooke, St. Louis, Mo.) mounted on the other optical port. The optical path utilizes a moveable mirror which can direct the collimated, polarized laser beam through focusing optics to form a spot, or a beam expander to form a large (>1 cm) beam whose central portion is roughly uniform over the field of view of the objective lens. Another movable mirror can direct the light either to the intensified CCD camera when using large area uniform illumination, or to the PMTs in the scanned spot mode. In spot scanning mode, a polarizing beamsplitter separates the parallel and perpendicular components of the emitted fluorescence and directs each to its designated PMT. An emission filter in the optical column rejects scattered excitation light from either type of detector. In CCD imaging mode, manually polarizers in the optical column of the microscope must be adjusted to obtain parallel and perpendicular images from which the fluorescence polarization or anisotropy can be calculated. A software program interfaces with data acquisition boards in a computer which acquires the digital output data from both PMTs and CCD. This program also controls the PMT power, electromechanical shutters, and galvanometer mirror scanner, calculates and plots fluorescence polarization in real time, and displays FP and intensity images. The images and data shown in FIG. 13 were acquired using a 2.5×objective lens, 30 mW laser power, and 100 msec integration time per point, at a resolution of 40×40 points in X and Y. The FP titration curve shown in FIG. 12C is for the thrombin-specific biosensor spot. The measured K_{d} value was approximately 15 nM in a biological fluid consisting of 10% human serum in phosphate buffered saline (PBS).

In another embodiment, the detection system is a single photon counter system (see, e.g., U.S. Pat. Nos. 6,016,195 and 5,866,348) requiring rastering of the sensor substrate to image larger areas and survey the different binding regions on the biosensor.

B. Detection to Targets of Physiological and Pharmacological Interest

Any optical method known in the art, in addition to those described above can be used in the detection and/or quantification of all targets of interest in all sensor formats, in both biological and nonbiological media. These targets include, e.g., those listed in the Table in Section 4Bii, entitled: “Exemplary pathway target molecules include, e.g.:”.

4. Methods of Using Nucleic Acid Sensor Molecules

A. Diagnostic Assays

The target activated biosensors according to the invention can be used to detect virtually any target molecule upon selection of the appropriate nucleic acid precursor molecule. In one embodiment, the target molecule is a target molecule associated with a pathological condition and detection of changes in the optical properties of the nucleic acid sensor molecules of the biosensor provides a means of diagnosing the condition. Target molecules which are contemplated within the scope include, e.g. proteins, modified forms of proteins, metabolites, organic molecules, and metal ions, as discussed above. Because signal generation in this system is reversible, washing of the biosensor(s) in a suitable buffer will allow the biosensor(s) to be used multiple times, enhancing the reproducibility of the any diagnostic assay since the same reagents can be used over and over. Suitable wash buffers include, e.g., binding buffer without target or, for faster washing, a high salt buffer or other denaturing conditions, followed by re-equilibration with binding buffer.

Re-use of the bio sensor is enhanced by selecting optimal fluorophores. For example, Alexa Fluor 488, produced by Molecular Probes, has similar optical characteristics compared to fluorescein, but has a much longer lifetime. However, in one embodiment, a site recognized by a nuclease is engineered proximal to the signal generating site, and sequences comprising signaling moieties are removed from the biosensor and replaced by new sequences, as needed.

i. Profiling Biosensors for Use in Diagnostic Assays

In one embodiment, the expression pattern of a plurality of target molecules is determined to obtain a profile of target molecules associated with a trait in an individual to determine an expression pattern which is diagnostic of that trait. In this embodiment, combinations of biosensors targeted to individual target molecules are selected until a signature optical profile is determined which is characteristic of a trait. Traits include, e.g., a disease, a genetic alteration, a combination of genetic alterations (e.g., a polygenic disorder), a physiological reaction to an environmental condition, or a wild type state (e.g., of an organism or of an organ system). The target molecules which generate the signature optical profile are identified (based on the type of biosensors used) as signature target molecules. The expression of the signature target molecules can thereafter be determined to identify the presence of the trait in a patient.

The expression of the target molecules can be identified using any molecular detection system known in the art; however, in a preferred embodiment, the detection system comprises nucleic acid sensor molecules and the trait is identified by detecting the signature optical profile. In one embodiment, data relating to the signature optical profile is stored in the memory of a computer. Signature optical profiles can be generated for individual patients or can be generated for populations of individuals. In the latter embodiment, data relating to a composite signature profile (e.g., comprising normalized data) is stored in the memory of a computer or in a computer program product.

Because the biological function of the target molecules does not need to be known, the biosensors according to the invention can be generated which are diagnostic of diseases/traits whose biological basis is not yet known or are the result of complex polygenic interactions and/or of environmental influences. In one embodiment, nucleic acid sensor molecules are identified which are activatable by synthetic polypeptides obtained from putative open reading frames identified in the human genome project and/or in other sequencing efforts. Combinations of these activatable nucleic acid sensor molecules (along with activatable nucleic acid sensor molecules specific for target molecules with known functions) are identified which generate a diagnostic optical signal, and signature target molecules are in turn identified which are linked to a particular trait, allowing a biological activity to be associated with a previously uncharacterized molecule.

Data relating to signature target molecules or to the optical signals generated upon activation of nucleic acid sensor molecules upon binding to signature target molecules is stored in a database, which can include further information such as sequence information or chemical structure information relating to the signature target molecule. A signature profile relating to a particular trait is generated based on normalized data from a plurality of tests. In one embodiment, a signature profile is obtained by determining any or all of the level, chemical structure, or activity, of signature target molecules associated with a disease in samples from a population of healthy individuals to determine a signature profile corresponding to a healthy state. In a further embodiment, signature profiles are obtained using data from subsets of populations which are divided into groups based on sex, age, exposure to environmental factors, ethnic background, and family history of a disease.

B. Drug Discovery

Generally, methods of drug discovery comprise steps of 1) identifying target(s) molecules associated with a disease; 2) validating target molecules (e.g., mimicking the disease in an animal or cellular model); 3) developing assays to identify lead compounds which affect that target (e.g., such as using libraries to assay the ability of a compound to bind to the target); 4) prioritizing and modifying lead compounds identified through biochemical and cellular testing; 5) testing in animal models; and 6) testing in humans (clinical trials). Through the power of genomics and combinatorial chemistry, large numbers of lead compounds can be identified in high throughput assays (step 3); however, a bottleneck occurs at step 4 because of the lack of efficient ways to prioritize and optimize lead compounds and to identify those which actually offer potential for clinical trials. The target activatable nucleic acid sensor molecules according to the present invention offer a way to solve this problem by providing reagents which can be used at each step of the drug development process. Most importantly, the target activatable nucleic acid sensor molecules according to the present invention offer a way to correlate biochemical data, from in vitro biochemistry and cellular assays, with the effect of a drug on physiological response from a biological assay.

In one embodiment of invention, a method for identifying a drug compound is provided, comprising identifying a profile of target molecules associated with a disease trait in a patient, administering a candidate compound to the patient, and monitoring changes in the profile. In another embodiment, the monitored profile is compared with a profile of a healthy patient or population of healthy patients, and a compound which generates a profile which is substantially similar to the profile of target molecules in the healthy patient(s) (based on routine statistical testing) is identified as a drug. In a further embodiment, both the profiling and the drug identification step is performed using at least one sensor molecule whose properties change upon binding to a target molecule.

In a further embodiment, a method for identifying a drug compound comprises identifying a plurality of pathway target molecules, each belonging to a pathway, monitoring the level, chemical structure, and/or activity of pathway target molecules in a patient having a disease trait, administering a candidate compound to the patient, and monitoring changes in the level, chemical structure, and/or activity of the pathway target molecules. In another embodiment, the monitored level, chemical structure, and/or activity of the pathway target molecules is compared to the level, chemical structure, and/or activity of pathway target molecules in a wild type patient or patients. In a further embodiment, both profiling and the identification of drug compounds is performed using at least one sensor molecule whose properties change upon binding to a pathway target molecule.

Properties according to this aspect include, e.g., optical properties, change in sequence, chemical structure, catalytic activity, and/or molecular weight. In a preferred embodiment, sensor molecules are target activated nucleic acid sensor molecules.

I. Target Activated Biosensors for Use in Identifying Lead Compounds

In one embodiment, biosensors activatable by signature target molecules, identified as described above, are provided and are validated by testing against multiple patient samples in vitro to verify that the optical signal generated by these molecules is diagnostic of a particular disease. Validation can also be performed ex vivo, e.g., in cell culture, (using microscope-based detection systems and other optical systems as described in U.S. Pat. Nos. 5,843,658, 5,776,782, 5,648,269, and 5,585,245) and/or in vivo, for example, by providing a profile biosensor in communication with an optical fiber.

The incorporation of biosensors into fiber optic waveguides is known in the art (see, e.g., U.S. Pat. Nos. 4,577,109, 5,037,615, 4,929,561, 4,822,746, 4,762,799, the entireties of which are incorporated by reference herein). The selection of fluorescent energy transfer molecules for in vivo use is described in EP-A 649848, for example. In this embodiment, nucleic-acid based biosensors are introduced into the body by any suitable medical access device, such as an endoscope or a catheter. The optical fiber is provided within a working lumen of the access device and is in communication with an optical imaging system.

In one embodiment, the same methods which are used to validate the diagnostic value of particular sets of target molecule/nucleic acid sensor molecule combinations are used to identify lead compounds which can function as drugs. Thus, in one embodiment, the effects of a compound on target dependent optical signaling is monitored to identify changes in a signature profile arising as a result of treatment with a candidate compound.

In one embodiment, samples from a treated patient are tested in vitro; however, samples can also be tested ex vivo or in vivo. When the diagnostic profile identified by the biosensor changes from a profile which is a signature of a disease to one which is substantially similar to the signature of a wild type state (e.g., as determined using routine statistical tests), the lead compound is identified as a drug. Target molecules which activate the biosensor comprise can comprise molecules with characterized activity and/or molecules with uncharacterized activity. Because large number of target molecules can be monitored simultaneously, the method provides a way to assess the affects of compounds on multiple drug targets simultaneously, allowing identification of the most sensitive drug targets associated with a particular trait (e.g., a disease or a genetic alteration).

Examples of suitable target molecules include, e.g., nuclear hormone receptor (NHR) polypeptides; G-coupled protein receptor (GPCR) polypeptides, phosphodiesterase (PDE).

NHR Polypeptides

Included in the invention are methods of identifying riboreporters such as allosteric ribozymes, signaling aptamers or aptamer beacons for detection of conformational isoforms of nuclear hormone receptors, as well as the riboreporters identified by the methods described herein.

Nuclear hormone receptors (NHRs) act as ligand-inducible transcription factors by directly interacting as monomers, homodimers, or heterodimers in complex with DNA response elements of target genes. The activation of these transcription regulators is induced by the change in conformation of the NHR upon complex formation with ligand.

Provided are methods for generating unique biosensors for each NHR ligand binding domain. The biosensors described herein can include, e.g., riboreporters such as allosteric ribozymes (ARs), including those derived from the hammerhead, hairpin, L1 ligase or group1 intron ribozymes and the like, or the riboreporter may be derived from aptamer beacons or signaling aptamers, any of which transduce molecular recognition into a detectable signal.

Also provided is a direct mechanistic assay for the action of small molecule ligand-agonism, -antagonism and partial antagonism of members of the NHR family. The mechanistic assays function in both in vitro biochemical as well as with in vitro cell-based settings. In the in vitro assay setting, the riboreporters (allosteric ribozyme, signaling aptamer, or aptamer beacon) are designed to recognize one conformational isomer of the NHR. In one embodiment, the riboreporter recognizes the unique conformation that exists for the agonist bound form of a hormone receptor; such as that observed for the estrogen receptor ligand binding domain ER_{(LBD)} when bound to estrogen [Shiau A K, Barstad D, Loria P M, Cheng L, Kushner P J, Agard D A, Greene G L. Cell. Dec. 23, 1998;95(7):927-37.] and then produces a detectable signal, such as release of fluorescently labeled oligonucleotide, radiolabeled oligonucleotide, or reveals a change in riboreporter conformation driven by ligand binding through a change in fluorescence or the like. Hence, in this embodiment, the riboreporter transduces molecular recognition of the ER-estrogen agonist complex into a detectable signal. The level of the signal is then used to quantify the amount of ER-estrogen agonist complex present in solution. In another embodiment, the ER-estrogen specific riboreporter is used as a screening tool in assays designed to detect inhibitors of ER-estrogen complex formation. These screening tools can be used to determine the inhibition potency of compounds in in vitro biochemical assays or in in vitro cell-based assays. Inhibitors of estrogen binding to ER_{(LBD)} are useful as anti-proliferative agents for treatment of breast cancers (e.g., tamoxifen) and other estrogen dependent diseases. In another embodiment, riboreporters are introduced into cell lines by known methods of transduction, transfection or coupling to peptide translocating agents such as tat or antenopeadia peptides. In another embodiment, the ER-estrogen complex specific riboreporter is an allosteric intron imbedded in a reporter gene such as GFP or luciferase or the like. When the intron derived riboreporter is expressed within the reporter gene it renders reporter gene expression effector dependent. Thus, in one embodiment functional GFP protein is expressed only when the ER-estrogen complex is present in the cell, and inhibitors of ER-estrogen complex formation thus block functional GFP protein expression in appropriate mammalian such as MCF7 or T47D, yeast or bacterial cell lines. In a further embodiment the MCF7 or T47D tumor cell lines transfected with GFP-ER-estrogen riboreporter sensitive construct are used to form tumor xenografts in nude mice. Thus, the transfected tumor xenograft cell lines can be used to form tumors in mice which are not only estrogen dependent but also regulate reporter gene expression in ER-estrogen dependent manner. These cell lines and tumor models are used to discover inhibitors of ER-estrogen complex formation in vivo.

NHR ligand binding domains bind antagonists and form additional conformational isomers. When antagonists are bound to the receptor a new conformer results such as that observed upon tamoxifen binding to the estrogen receptor to form a stable ER-tamoxifen complex [Shiau A K, Barstad D, Loria P M, Cheng L, Kushner P J, Agard D A, Greene G L. Cell. Dec. 23, 1998;95(7):927-37.]. Accordingly, the invention includes use of an ER-tamoxifen specific riboreporter that is used to detect the levels of antagonist specific complex in both in vitro biochemical, cell-based and, in in vivo assays as described above.

Riboreporters can be developed that are specific for the ligand binding domains of all NHRs. In addition, it should be clear that riboreporters for agonist, antagonist, dimeric or multimeric forms of NHR LBDs can be used to screen for inhibitors of LBD function and therefore for inhibitors of NHR dependent transcriptional activation or repression. It should be clear to one skilled in the art that riboreporters specific for individual LBD complexes can be used to screen for agents that modify NHR function in in vitro and in in vivo assays.

NHRs are multidomain proteins containing a variable NH2-terminal region (A/B), a conserved DNA binding domain (DBD) or region C, a linker region D, and a conserved region E that contains the ligand biding domain (LBD). NHRs also contain regions required for transcriptional activation, of particular interest is the region AF-2 which is located in the COOH-terminus and whose function is strictly ligand dependent. Provided herein is a method for generating unique riboreporters to each of the 63 known human NHRm LBDs. In addition, methods are described that enable the generation of riboreporters capable of recognizing the activated state of the NHR by selection for riboreporters geometries which signal the presence of either the activated or inactivated conformation (NHR with bound ligand), but whose signaling action is quiescent in the presence of other forms of the NHR.

The riboreporters allow the direct, simultaneous, and rapid detection of the activation states of all NHRs. This tool can be used in in vitro assays for receptor activation with agonists and antagonists, and can be used to generate cell lines and animal models that report on the activation state of such receptors in a biological setting and as a function of drug or drug lead.

GPCR Riboreporters

Also provided by the invention are riboreporters such as allosteric ribozymes, signaling aptamers, or aptamer beacons for detection of conformational isoforms of G-protein coupled receptors.

G-protein coupled receptors (GPCRS) play fundamental roles in regulating the activity of virtually every cell. Upon binding of extracellular ligands, GPCRs interact with a specific subset of heterotrimeric G-proteins that can then, in their activated forms, inhibit or activate various effector enzymes and/or ion channels. Molecular cloning studies have identified over multiple human GPCRs, and have identified the ligands for many of these.

GPCRs include three domains: an extracellular N-terminus, a central domain of seven trans-membrane helices, and a cytoplasmic C-terminus. Activation of GPCRs is induced by ligand binding, which causes a conformational change in the receptor and exposes cytoplasmic helices II and III, as well as helix VII. The invention provides a method to generate unique biosensors for each GPCR. The biosensors described herein include riboreporters such as allosteric ribozymes (AR), such as those derived from the hammerhead, hairpin, L1 ligase or group 1 intron ribozymes and the like, or the riboreporter may derived from aptamer beacons or signaling aptamers, and of which transducer molecular recognition into a detectible signal.

Upon the activation by an extracellular ligand or stimuli, G-protein coupled receptor(GPCR) polypeptides activate intracellular Gα-protein. A GPCR can activate a number of Gα-proteins. For example, adrenergic receptors activate Gi, which inhibit adenylyl cyclases, Gs, which stimulate adenylyl cyclases, and Gq, which regulate cellular Ca ion level(Wenzel-Seifert and Seifert 2000). Thus, it is highly desirable to distinguish the class of Gα-proteins which are activated through the GPCR of interest in cell.

The initial drug screening of the GPCRs is normally performed by competition assay with radiolabeled ligands. For a cell based GPCR assay, incorporation of radiolabeled GTP can be measured to detect the coupling of Gα-protein and GPCR, however this assay does not distinguish the type of Gα-proteins involved. The assays for the effect on individual effectors, such as the Ca⁺⁺ flow or cellular cAMP level, are also used, but only the selected downstream signal can be measured at time.

Upon activation, Gα-protein goes significant conformational change which results in release of GDP and association with GTP (Coleman and Sprang 1998). It also dissociates from its βγ-subunits. This activated form of Gα-protein then becomes capable of interacting with its effector(Li, Stemweis et al. 1998). The well-characterized conformation change takes place in three swtiches; switch I (residues 177-187 in Giα1), switch II (residues 199-219 in Giα1), and switch III (residues 231-242 in Giα1). The sequences and the conformational changes in these switches are well conserved among Gα-proteins.

Ras is a member of small GTPase protein, which shares significant similarity. GTP-bound ras and GDP-bound ras can be distinguished by RBD (ras binding domain) of Raf-1 (Taylor, Resnick et al. 2001). The activated state of Rap1 can be identified by RalRDS (Franke, Akkerman et al. 1997). This indicates significant change in the surface of the protein, and the effector binding surface are only available for interaction in GTP complex form.

The invention provides methods for selecting riboreporters which recognize the conformational change upon GTP binding and/or specifically interact with newly exposed G-protein effector binding sites upon the activation. Class-specific activated Gα-protein riboreporters recognize the activated Gα-proteins or its effector binding site, which allow us to interpret the multiple type of downstream signal affect by the GPCR. It can used in both in vitro HTS and cell-based HTS.

Also described is a method for developing a direct mechanistic assay of the action of small molecule ligand-agonism, -antagonism, and partial antagonism of members of the GPCR family. The mechanistic assays function in both in vitro biochemical and in vitro cell-based settings. In the in vitro assay setting, the riboreporters (allosteric ribozyme, signaling aptamer, or aptamer beacon) are designed to recognize one conformational isomer of the GPCR.

In one embodiment, the riboreporter recognizes the unique conformation that exists for the activated state when in complex with ligand; such as that observed for the beta-2 adrenergic receptor when in complex with the artificial ligand isoproterenol (Ghanouni et al., PNAS USA, 98:5997-6002(2001)) and then produces a detectable signal, such as release of fluorescently labeled oligonucleotide, radiolabeled oligonucleotide, or reveals a change in riboreporter conformation driven by ligand binding through a change in fluorescence or the like. Hence, in this embodiment, the riboreporter transduces molecular recognition of the beta-2 adrenergic receptor—in complex with epinephrine, norepinephrine or an artificial ligand such as isoproterenol into a detectible signal. The level of the signal is then used to quantify the amount of beta-2 adrenergic receptor-agonist complex present in solution.

In another embodiment, the beta-2 adrenergic-agonist riboreporter is used as a screening tool in assays designed to detect agonists of the beta-2 adrenergic receptor. These screening tools can be used to determine the activation potency of compounds in in vitro biochemical assays or in in vitro cell-based assays. Agonists of the beta-2 receptor are useful in the treatment of asthma (Robinson, et al. Lancet 357:2007-2011(2001)). In another embodiment, riboreporters are introduced into cell lines by known methods of transduction, transfection, or coupling to peptide translocating agents such as tat or antennapoedia peptides.

In another embodiment, the beta-2 adrenergic receptor-agonist complex specific riboreporter is an allosteric intron imbedded in a reporter gene such as GFP or luciferase or the like. When the intron-derived reporter is expressed within the reporter gene it renders reporter gene expression effector dependent. Thus, in one embodiment functional GFP protein is expressed only when the beta-2 adrenergic receptor-agonist complex is present in the cell, and inhibitors of beta-2 adrenergic receptor-agonist complex formation this block functional GFP protein expression in appropriate cells such as mammalian human peripheral blood leukocytes, yeast, insect, or bacterial cell lines.

In a further embodiment, Chinese hamster fibroblasts, which do not express beta-adrenergic receptors (Sheppard, et al., PNAS USA 80:233-236(1983)), are transfected with both the riboreporter and the gene coding for the beta-adrenergic receptor under a constitutive promoter, are used to create a model cell line suitable for HTS screening of candidate beta-2 agonists. Furthermore, cells can be caused to express known allelic variants, such as gln27-to-glu associated with obesity (Large, et al., J Clin. Invest 100:3005-3013), to create cells lines which model specific disease states. Furthermore, chimeric mice can be created by “knock-in” (Monroe et al., Immunity 11:201-212(1999)) which will express the riboreporter in every cell as the result of blastocyst fusion (Chen et al., PNAS USA 90:4528-4532(1993), and used for pharmokinetic or bioavailability studies in which the GPCR activation states of various tissues in the organism are of concern.

GPCRs bind antagonists, which cause the GPCRS to become resistant to conformational changes, or result in conformations not susceptible to activation, or blockade the ligand binding domain from interaction with the appropriate ligand and thus prevent activation of the GPCR, such as the beta-2 adrenergic receptor antagonist butoxamine (Horinouchi et al., Pharmacology 62:98-102(2001)). Hence, the invention also provides a method for using a riboreporter to detect conformers which result from binding of GPCRs to antagonists. Furthermore, when the cell line described above is transfected with a mutant variants of GPCRs which spontaneously adopt the active conformation, such as lys272-to-ala (Pei,et al., PNAS USA 91:2699-2702(1994) and references therein) the riboreporter can be employed in a screen for compounds which are beta-2 antagonists (Ramsay et al, Br J Pharmacol 133:315-323(2001)). Antagonists of the beta-2 receptor are useful in the treatment of cardiovascular diseases (Nagatomo, et al., Cardiovasc Drug Rev 19:9-24(2001)). The invention accordingly provides a method for using a Beta-2 adrenergic receptor—butoxamine complex-specific riboreporter that is used to detect the levels of an antagonist specific complex in both in vitro biochemical, cell-based, and in vivo assays as described above.

Riboreporters can also be developed that are specific for the occupancy state of the ligand-binding domains of all GPCRs. In addition, riboreporters for the agonist, antagonist, dimeric, or multimeric forms of all GPCRs can be used to screen for inhibitors or activators of GPCR function and therefore for inhibitors or activators of GPCR-dependent cell signaling pathways. Riboreporters specific for individual GPCR complexes can be additionally be used to screen for agents that modify GPCRs in in vitro and in vivo assays.

GPCRs are broadly divided into three domains, an extracellular N-terminal, a cytoplasmic c-terminal, and a central domain with seven transmembrane helices connected by unstructured loops. Upon GPCR activation, loops C-II and C-III, and helix VII become cytoplasmically accessible. The methods described herein allow for generating unique riboreporters to any GPCRs In addition, methods are described that enable the generation of riboreporters capable of recognizing the activated state of the GPCR by selecting for riboreporter geometries which signal the presence or absence of the activated conformation of the receptor through recognition of one or all of the mobile domains, but whose signaling action is quiescent in the presence of other forms of the GPCR.

Phosphodiesterase-specific Riboreporters

Multiple classes of phosphodiesterases have been identified in humans. These enzymes catalyze a reaction that converts second messenger cAMP and cGMP into 5′-AMP and 5′-GMP. Different class of PDEs have different substrate specificity as well as different physiological function. For example, PDE4s are specific for cAMP and PDE5 are specific for cGMP.

The invention provides multiple classes of PDE riboreporters. The first class of riboreporters can distinguish cAMP vs. 5′AMP (cGMP vs 5′GMP) (Koizumi, Kerr et al. 1999) (Koizumi, Soukup et al. 1999). The second class of riboreporter binds to the active site of PDE in a class specific manner and inhibits PDE catalytic activity. This class of riboreporter can be raised using PDEs in the presence and absence of high affinity known inhibitors (e.g. Ropalim for PDE4). The third class of riboreporter recognizes PDE in a class-specific (e.g. PDE1-11) or subclass-specific (PDE4A-D) manner.

Protein Kinase-specific Riboreporters

The invention also provides riboreporters raised against protein kinases. In one embodiment, the invention provides riboreporters that are sensitive to the phosphorylation state in a given peptide sequence. Alternatively, native proteins can be used with different phosphorylation states in order to raise riboreporters that are sensitive to phosphorylation state. For example, ERK1/2 and phosphorylated ERK1/2 can be distinguish by specific riboreporters (Seiwert, Stines Nahreini et al. 2000). The riboreporter also can be competitive inhibitor for kinase by binding at ATP or substrate binding sites.

Alternatively, an ADP-dependent riboreporter can be obtained at lower pH. These riboreporters can be used to detect the production of ADP.

ii. Pathway Profiling Biosensors

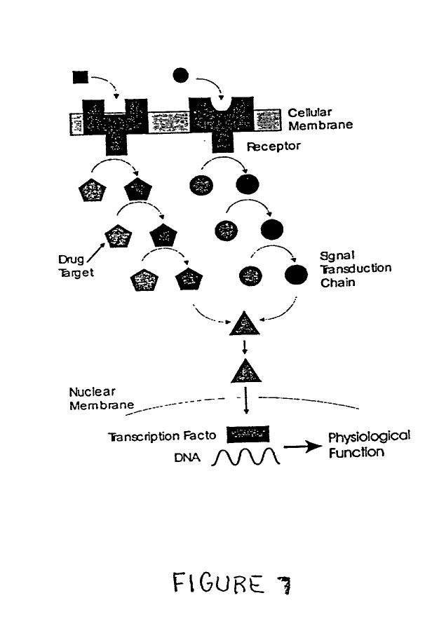

As shown in FIG. 7, physiological function is modulated by complex pathways, each of which may have multiple overlapping and intersecting steps. Furthermore, the proteins involved in these pathways are highly homologous and can have overlapping substrates and drug specificities. Using current techniques, it is possible only to monitor the response of single elements of a pathway. These techniques are inadequate to understand the mechanism of drug interactions. For example, a particular drug found to have a particular in vitro activity against a single target in biochemical assays might interact with other proteins in the same pathway, or in other unrelated pathways. Consequently, physiological function is often uncorrelated with the results of biochemical assays of a single target.

The nucleic acid sensor molecules according to the invention provide reagents to simultaneously quantify the level and chemical state of all components in a molecular pathway As used herein, “pathway target molecules” are target molecules involved in the same pathway and whose accumulation/activity! and/or chemical structure is dependent on other pathway target molecules, or whose accumulation/activity/and/or chemical structure affects the accumulation/activity and/or chemical structure of other pathway target molecules. Pathway target molecules according to the invention include, e.g., proteins, such as enzymes, modified forms of proteins, such as phosphorylated, sulfated, ribosylated proteins, methylated proteins (Arg, Asp; N, S or O directed), prenylated proteins (such as by farnesyl, geranylgeranyl, and other types of prenylation) acetylated or acylated proteins, cleaved or clipped proteins, bound or unbound forms of proteins, allelic variants of a protein (e.g., proteins differing from each other by single amino acid changes in a protein), as well as substrates, intermediates, and products of enzymes (including both protein and non-protein molecules).

In another embodiment, signature pathway target molecules are identified by pre-selecting a plurality of nucleic acid sensor molecules activatable by pathway-specific target molecules. In one embodiment, a pathway profiling biosensor is provided comprising at least one nucleic acid sensor molecule specific for every molecular species within a pathway (e.g., a signaling pathway), to generate a biosensor which can monitor the levels, chemical structure, and/or activity of every molecular species in the pathway.

Because of the finite number of target molecules (as determined from data obtained from the Human Genome Project) and the high throughput of the biosensors of the instant invention (greater than 10,000 target molecules can be monitored simultaneously), the pathway profiling 10 biosensors of the instant invention make it feasible to evaluate the response of all the components of a pathway to a drug compound simultaneously.

In one embodiment, a pathway profiling biosensor reactive to the components of an entire pathway, is contacted with a sample from a patient having a disease, and an optical signal corresponding to a disease state is determined to identify signature pathway target molecules which are diagnostic of that disease. Samples from a plurality of patients are obtained and tested using the pathway profiling biosensor to identify a pathway profile that is diagnostic of the disease, the pathway profile comprising normalized data relating to any. or all of. the level, structure, and activity, of the signature pathway molecules. A pathway profile corresponding to a wild type state is determined by testing the pathway profiling biosensor molecules against samples from a population of healthy patients , or subsets of populations of healthy patients. In one embodiment, data relating to the optical signals generated by nucleic acid sensor molecules activated by the signature pathway target molecules is stored within the memory of a computer or within a computer program product.

The pathway profiles can be used in diagnostic testing as discussed above. In one embodiment, a pathway profiling biosensor is used in which the pathway is one which is known or suspected of being disrupted in patients having a particular trait (e.g., having a particular disease or genetic alteration(s)). For example, in one embodiment, one pathway profiling biosensor used to evaluate samples from a patient with cardiovascular disease is a cholesterol metabolism pathway profiling biosensor. However, random combinations of pathway profiling biosensors can be used to assess the physiological state of a patient, to identify signature pathway profiles which are diagnostic of diseases whose molecular basis has not yet been identified or characterized.

In one embodiment, pathway profiling biosensors according to the invention are used to assess the affect of a candidate drug on any or all of the level, chemical structure, or activity of signature pathway target molecules to generate a drug treatment pathway profile. In this embodiment, a pathway profiling biosensor is contacted with a sample from a cell or physiological system (e.g., a group of cells, a tissue system, an organ system, or a patient), and changes in optical signals are obtained which are correlated any, or all of, the level, chemical structure, or activity of a particular pathway target molecule by relating the optical signal obtained to the address of the nucleic acid sensor molecule, as described above. In one embodiment, a drug treatment profile which is substantially similar to a signature pathway profile obtained from a healthy population of patients (as determined by observing no significant differences in the profile by routine statistical testing, to within 95% confidence levels) is used to identify a candidate drug as one which is suitable for further testing. The profile produced by such a drug is used to produce an effective drug treatment profile, against which other candidate drugs can be compared.

In another embodiment, a candidate drug is tested against a plurality of pathway biosensors including the one which will generate a diagnostic signature profile, to identify drugs which produce an effective drug treatment profile without effecting significant changes in other pathway profiles. In this embodiment, the systemic effects of a candidate drug can be predicted.

In further embodiments, it is desirable to use a biosensor representing less than an entire pathway. In one embodiment, a biosensor is provided comprising nucleic acid sensor molecules specific for signature pathway target molecules. In a further embodiment, a biosensor is provided which comprises nucleic acid sensor molecules necessary to evaluate particular components of a pathway suspected of being involved in a disease. For example, compounds being screened to identify candidate drugs that affect ameliorate diseases relating to defective DNA repair can be tested against a pathway biosensor comprising only S phase cell cycle target molecule reactive nucleic acid sensor molecule.

Exemplary pathway target molecules include, e.g.:

| | |
|---|---|
| apoptotic pathways | Bcl, Bak, ICE proteases, Ich-1, CrmA, CPP32, APO-1/Fas, |
| | DR3, FADD containing proteins, perform, p55 tumor necrosis |
| | factor (TNF) receptor, NAIP. TAP, TRADD-TRAF2 and |
| | TRADD-FADD, TNF, D4-GDI, NF-kB, CPP32/apopain, |
| | CD4O, IRF-i, p53, apoptin |
| blood clotting pathways | thrombin, fibrinogen, factor V, Factor VIII- FVa, FVIIIa, |
| | Factor XI, Factor Xia, Factors IX and X, thrombin receptor, |
| | thrombomodulin (TM), protein C (PC) to activated protein C |
| | (aPC). aPC, plasminogen activator inhibitor-i (PAT-i), tPA |
| | (tissue plasminogen activator) |
| calcium signaling pathways | calmodulin, calcineurin, |
| Cell cycle | |
| G0 | MPS, CYTOSTATIC FACTOR (CSF) (INCLUDING MOS) |
| Pathway | |
| G1 | mid GI phase: cdk4/cycin Dl-3 and cdk6/cyclin Dl-3 |
| | late G 1 phase: cdk2/cycin E |
| | others: p53, p21, p16, Rb, p2′1, E2F, Cdc25A, Cdc25B |
| S | cyclin A/CDK2, cyclin B/Cdc2, SPA-i, Cdc25A, Cdc25B |
| G2 | G2/M transition phase: cdkl/cyclin B 1-3, cdkl/cyclin A, |
| | Cdc25A, Cdc25B, Cdc25C. PIN!, Chki, Myt 1, Wee 1 |
| M | Cdc2/cyclin B, P1k, Cdc25C, |
| Cholesterol metabolism pathway | LDL, LDL-receptor, VLDL, HDL, cholesterol acyltransferase, |
| | apoprotein E, Cholesteryl esters, ApoA-I and A-I1, HMGCoA |
| | reductase, cholesterol |
| Flt-3 pathway | flt-3 pathway flt-3, GRP-2, SHP-2, SHIP, She |
| JAK/STATS signaling pathway | Jak1, Jak2, IL-2, IL-4 and IL-7, Jak3, Ptk-2, Tyk2, EPO, GH, |
| | prolactin, IL-3, GM-CSF, G-CSF, IFN gamma, LW, OSM, IL- |
| | 12 and IL-6, IFNR-alpha, IFNR-gamma, IL-2R beta, IL-6R, |
| | CNTFR, Stat 1 alpha, Stat 1 beta, and Stats2-6 |
| MAP kinase signaling pathways | flt-3, ras, raf, Grb2, Erk-i, Erk-2, and Src, Erb2, gpl3O, MEK-1, |
| | MEK-2, hsp 90, JNK, p38, Sin!, Styi/Spcl, MKK's, MAPKAP |
| | kinase-2, TNKISAPK |
| P53 pathway | bax, bid, caspases, cytochrome c |
| PI 3 kinase athway | SHIP, Akt |
| ras activation pathways | p120-Ras GAP, neurofibromin, Gap1, Ra!-GDS, Rsbs 1, 2, and |
| | 4, Rinl, MEKK- 1, and phosphatidylinositol-3-OH kinase |
| | (P13K), ras |
| SIP signaling pathways | GRB2, SIP, ras, P1 3-kinase |
| SHC signaling pathways | trkA, trkb, NGF, BDNF, NT-4/5, trkC, fNT-3, Shc, PLC |
| | gamma 1, P1-3 kinase, SNT, ras, rafi, MEK and MAP kinase |
| TGF-13 signaling pathways | BMP, Smad 2, Smad4, activin, TGF |
| T-cell receptor complex | lck, fyn, CD4, CD8, T cell receptor proteins |
| MHC-I pathways | TAP proteins, LMP 2, LMP 7, gp 96, HSP 90, HSP 70 |

If desired, target activatable sensor molecules (also referred to herein as riboreporters) can be raised against particular amino acid sequences in the polypeptides. Some representative peptide regions are presented below.

| | |
|---|---|
| Sequence | enzyme |

| | |
|---|---|
| (SEQ ID NO:52) | PKA |
| L-R-A-S-L-G | |
| (SEQ ID NO:53) | PKC |
| A-A-K-I-Q-A-S-F-R-G-H-M-A-R-K-K | |
| (SEQ ID NO:54) | cdc2 |
| P-K-T-P-K-K-A-K-K-L | |
| (SEQ ID NO:55) | DNA-PK |
| E-P-P-L-S-Q-E-A-F-A-D-L-W-K-K | |
| (SEQ ID NO:56) | CK-1 |
| D-D-D-E-E-S-I-T-R-R | |
| (SEQ ID NO:57) | CK-2 |
| R-R-R-E-E-E-T-E-E-E | |
| (SEQ ID NO:58) | CaM KII |
| K-K-A-L-R-R-Q-E-T-V-D-A-L | |
| (SEQ ID NO:59) | P38 |
| S-T-K-V-P-Q-T-P-L-H-T-S-R-V | |
| (SEQ ID NO:60) | PKA |
| R-R-R-R-S-I-I-F-I | |
| (SEQ ID NO:61) | PKCα |
| R-R-R-R-R-K-G-S-F-R-R-K-A | |
| (SEQ ID NO:62) | PKCβI,II |
| R-K-L-K-R-K-G-S-F-R-R-K-A | |
| (SEQ ID NO:63) | PKCγ |
| R-R-R-R-R-K-G-S-F-K-K-F-A | |
| (SEQ ID NO:64) | PKCδ |
| A-A-R-K-R-K-G-S-F-F-Y-G-G | |
| (SEQ ID NO:65) | PKCε |
| Y-Y-X-K-R-K-M-S-F-F-E-F-D | |
| (SEQ ID NO:66) | PKCη |
| A-R-L-R-R-R-R-S-F-R-R-X-R | |
| (SEQ ID NO:67) | PKCζ |
| R-R-F-K-R-Q-G-S-F-F-Y-F-F | |
| (SEQ ID NO:68) | PKCμ |
| A-A-L-V-R-Q-M-S-V-A-F-F-F | |
| (SEQ ID NO:69 | CaM KII |
| K-R-Q-Q-S-F-D-L-F | |
| (SEQ ID NO:70) | Phosphorylase |
| F-R-M-M-S-F-F-L-F | kinase |
| (SEQ ID NO:71 | SLK1 |
| R-R-F-G-S-L-R-R-F | |
| (SEQ ID NO:72) | SRPK2 |
| R-R-R-H-S-R-R-R-R | |
| (SEQ ID NO:73) | AKT/PKB |
| R-K-R-X-R-T-Y-S-F-G | |

In one embodiment, a pathway biosensor array is generated comprising target activatable nucleic acid sensor molecules which are activatable by components of a cell cycle pathway. In this embodiment, a cell cycle biosensor is generated comprising nucleic acid nucleic acid sensor molecules activatable by at least two members selected from the group consisting of: MPS, Cytostatic factor (CSF) (including Mos), cdk4, cyclins D1-3, cdk6, cdk2, cyclin E, p53, p21, p16, Rb, p2′7, E2F, cyclin A, cyclin B, cdk1, cyclin B1-3, Cdc2, SPA-1, and other biomolecules involved in cell cycle regulation.

In another embodiment, the cell cycle biosensor array generated is used to evaluate 10 samples from patients suspected of having a disorder affecting cell proliferation (e.g., cancer) and a signature target molecule profile is determined which is diagnostic of this disorder. Changes in the signature target molecule profile upon treatment with a candidate compound are subsequently monitored by any or all of in vitro, ex vivo, and in vivo methods, as described above, to identify and/or validate lead compounds for use in cancer therapies.

In further embodiments, a cell cycle biosensor is provided comprising a plurality of locations, each location comprising a set of nucleic acid sensor molecules activatable by target molecules which identify a different portion of the cell cycle. Thus, in one embodiment, a cell cycle biosensor comprises at a first location, nucleic acid sensor molecules activatable by G0 specific target molecules (e.g., MPS, Cytostatic factor (CSF) (including Mos)), at a second location, nucleic acid sensor molecules activatable by G1 specific target molecules (cdk4, cyclin D1-3, cdk6, cdk2, cyclin E, p53, p21, p16, Rb, p27, E2F), at a third location, nucleic acid sensor molecules which are activatable by S specific target molecules (e.g., cyclin A/CDK2, cyclin B/Cdc2, SPA-), at a fourth location, nucleic acid sensor molecules activatable by G2 specific target molecules (e.g., cdk1, cyclin B 1-3, cyclin A), and at a fifth location, nucleic acid sensor molecules activatable by M specific target molecules (e.g., Cdc2, cyclin B). In this way the effects of diseases and/or drugs on specific phases of the cell cycle can be assessed.

Similarly, pathway specific biosensors can be generated for any of apoptotic pathways, blood clotting pathways, calcium regulation pathways, cholesterol metabolism pathways, the fit-5 3 pathway, JAKISTATS signaling pathway, MAP kinase signaling pathways, p53 pathway, P1 3 kinase pathway, ras activation pathways, SIP signaling pathways, SHC signaling pathways, TGF-13 signaling pathways, T-cell receptor complex, and MHC-I pathways, using exemplary target molecules listed above, or other target molecule components of the respective pathways.

It should be apparent to those of ordinary skill in the art, that many other pathways exist whose components have been characterized and that target molecules within these pathways are also encompassed within the scope (e.g., including, but not limited to, phosphatase pathways, transcription factor pathways, hormone dependent pathways, as well as intermediary metabolism pathways, and developmental pathways). Further, additional pathways can be identified using the nucleic acid based biosensor profiling techniques discussed above (e.g., identifying pathway molecules involved in the functioning of a wild type or diseased organ system, such as the cardiovascular system, central nervous system, digestive system, reproductive system, pulmonary system, skin system, and the like), and these also are encompassed within the scope of the invention.

Alternatively, or additionally, pathway specific molecules can be identified by other techniques known in the art (see, e.g., U.S. Pat. Nos. 6,087,477, 6,054,558, 6,048,709, and 6,046, 165) and used to engineer additional pathway target activatable nucleic acid sensor molecules. Because there is a finite number of pathway target molecules in each pathway (constrained by the absolute number of gene products which have been identified) (see, e.g., Drews, Science 287: 1960-1964), it is feasible using the target activatable nucleic acid sensor molecules to generate biosensors representative of an entire pathway.

In further embodiments, sets of pathway biosensors are used to monitor the expression/activity of target molecules representing complex systems. Thus, for example, the effect of target molecules on the cardiovascular system and pulmonary system can be monitored simultaneously. In one embodiment, an array representative of a plurality of systems in the human body is used in methods to assess the effects of drug compounds on multiple systems in the body.

iii. Using Pathway Nucleic Acid Sensor Molecules in Drug Optimization

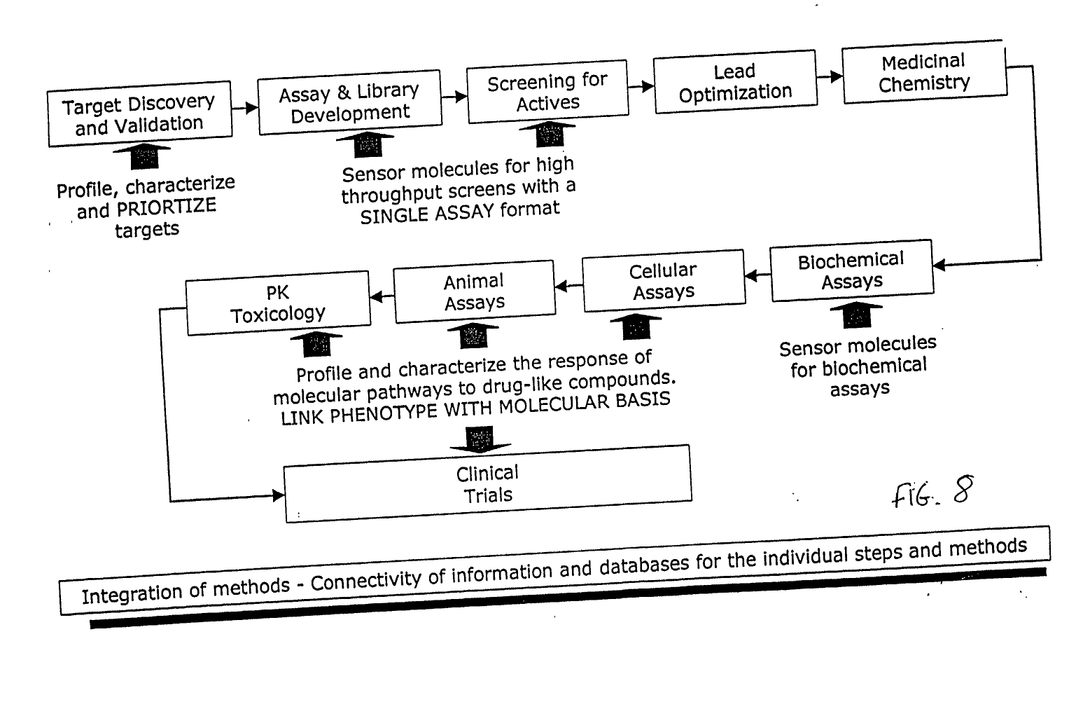

The pathway nucleic acid sensor molecules according to the invention can be used in every step of a drug optimization process, as shown in FIG. 8, and are suitable reagents for use in conventional high throughput screening systems making them extremely adaptable for use alone, or in conjunction with, other drug development assays.

Step 1. Drug Target Discovery or Drug Target Validation

As discussed above, pathway nucleic acid sensor molecules can be used to identify signature target molecules which are diagnostic of particular traits, such as disease. Signature target molecules are drug targets whose levels, structure, and/or activity can be used to evaluate the efficacy of compounds. A large number of signature drug targets, both characterized and uncharacterized, can be identified simultaneously using a single pathway biosensor according to one embodiment. In one embodiment, a pathway biosensor recognizes and be independently activated by about 1-5,000 molecules. In another embodiment, a pathway biosensor recognizes and be independently activated by about 500-10,000 molecules, and in one embodiment, by greater than 10,000 molecules.

Step 2. High Throughput Screening

In one embodiment, the drug targets identified in step 1 are evaluated in high throughput screening assays, using either solution-based biosensors or substrate-based biosensors, to characterize the biological activity of a drug target. For example, in one embodiment, nucleic acid sensor molecules are used to assess levels of substrate, product and intermediates produced by an enzyme in a wild type vs. a disease state, to identify other components of a pathway that would be affected by a drug acting on that target (i.e., secondary drug targets). In another embodiment, the levels, structure, and/or activity of all of the modified forms of a drug target, or the active and inactive forms of a drug target (e.g., a receptor) is determined in a wild type vs. a disease state, to further develop a diagnostic profile of a signature pathway target molecule and to evaluate changes of that profile in the presence of a drug.

In a further embodiment, the same type of pathway biosensor used to identify a diagnostic profile is contacted with samples from patients exposed to a compound. A compound-treated sample which produces substantially similar levels, structure, and/or activity of target and secondary drug targets in a sample from a healthy patient is used to identify a compound as a candidate drug. Because this testing is done in a high throughput format, a single dose of a candidate drug is evaluated in any given test.

Step 3. In vitro Biochemical Assays

In one embodiment, the nucleic acid sensor molecules used in step 2, are tested in an in vitro biochemical assay to determine compound potency. In this embodiment, a preliminary dosing effect is determined to identify the IC50 of candidate drug. In one embodiment, multiple biosensors of the type used in step 2 are contacted with samples from patients exposed to different doses of the candidate drugs identified in step 2, to identify candidate drugs with the highest potency (e.g., requiring the least amount of drug to generate a wild type profile or an effective drug profile.

Step 4. Cellular Assays

In one embodiment, nucleic acid sensor molecules are used in cellular assays where the effect of adding a compound on cell physiology is known and the researcher wants to determine that the drug is in fact acting through the drug target selected in steps 1-3. Here a candidate drug is added to a physiological system (e.g., cell(s), tissue(s), organ(s), or a patient). Cells from the physiological system are lysed and the substrate or product of an enzyme reaction is monitored using the nucleic acid sensor molecule either in an ELISA format or other solid support-based format (e.g., a pathway profiling array) or a solution phase format. In another embodiment, cell lysates are contacted with a pathway profiling biosensor specific for a target or pathway of interest to determine the profile of target molecules in the lysed sample. The profile is then compared to the wild type profile and the disease profile to determine if the drug is operating in vivo to restore a cell to its wild type state. Thus, the physiological effect of a candidate drug on a physiological system is correlated with the in vivo mechanism of action of the candidate drug.

In a preferred embodiment, molecular pathway profiling arrays comprised of nucleic acid sensor molecules affixed to a solid support are used in cellular assays to determine the selectivity of a compound for one target in a pathway relative to other candidate targets in a signal transduction pathway(s) or in another biochemical pathway(s). This data can be used to validate a drug lead or drug target.

In one embodiment, nucleic acid sensor molecules are expressed in vivo or intracellularly using plasmids, viruses or other extra-chromosomal DNA vectors and the cellular nucleic acid sensor molecules are extracted and used to determine the activity of a drug or drug target. These cellular assays can also determine the selectivity of a compound for one target in a pathway relative to other candidate targets in a signal transduction pathway(s) or in another biochemical pathway(s). This data can be used to validate a drug lead or drug target.

In vivo Detection:

With (Amersham) SPA scintillant beads coupled to nucleic acid sensor molecules, can look at cellular processes in situ in real time, by culturing cells directly onto a microtiter plate and allowing uptake of scintillant beads and radioisotope by cells. Can then monitor biosynthesis, proliferation, drug uptake, cell motility, etc. via luminescence generated by beads in presence of selected target.

Step 5. Medicinal Chemistry

In one embodiment, drug-lead potency, specificity, and/or in vivo activity is optimized by an iterative repetition of any or all of steps 1-4. In one embodiment, steps 1-4 are repeated until the desired potency, selectivity and in vivo mechanism of action of a candidate drug is obtained. Potency can range from picomolar affinity to nanomolar affinity as measured by in vitro 1C50 values. The desired selectivity of a drug candidate for its target can vary from 2 to a million-fold, and can be obtained by measuring the potency (IC50) of a drug lead toward the drug target, versus the drug's potency (1C50) values against other pertinent targets (target pertinence is determined by the requirements of the biological system under investigation). A drug lead is deemed optimal when the parameters of potency, selectivity and cellular action are optimized with respect to each other.

In another embodiment, known drug leads from Step 6 are found to be specific for targets that were not known to the researcher in step 2. This is also termed target discovery and validation, and occurs when steps 1-4 are repeated in an iterative fashion of any or all steps and the drug target is identified by the pathway profiling array to, in fact, exist in an alternative signal transduction pathway, or to be a novel protein or enzyme in the pathway originally under investigation. Thus, MPP arrays can identify the site of action of a drug lead, and can determine the relative selectivity of a drug for one drug target of drug target pathway.

Step 6. Animal Model Assays

In this embodiment, a target cells (e.g., tissue(s)) are removed from an animal model of the disease being targeted for treatment and lysed for testing. The lysate is contacted with nucleic acid sensor molecules either in a solid phase assay, a solution phase assay, or in a pathway profiling biosensor array format to assess the in vivo biological activity of a candidate drug identified by any of the previous steps or by some other method, on a target or pathway. Thus, in this embodiment, the physiological effect of a drug on a diseased or normal tissue is correlated with the in vivo mechanism of action of the drug.

Step 7. Optimization of the Drug Lead

In one embodiment, drug-lead potency, specificity, and/or in vivo activity are optimized by an iterative repetition of any or all of steps 1-6. In one embodiment, steps 1-5 are repeated until the desired potency, selectivity and in vivo mechanism of action of a candidate drug are obtained.

Step 8. Pharmacokinetic Studies

In one embodiment, the nucleic acid sensor molecules are used in pharmaco-kinetic studies, where the effect of a drug on the physiology of a cell, group of cells, tissue(s), organ(s), or animal model is assessed by obtaining blood, plasma, tissue, or a cell, and contacting this material with nucleic acid sensor molecules either in a solid phase (e.g., ELISA), solution or array format to assess the in vivo pharmacological or toxicological activity of a compound. In this embodiment, the nucleic acid sensor molecules used are developed against the candidate drug itself, its metabolic products, and! or the metabolic products of proteins and small ligands involved in a xenobiotic or topological response to drug treatment.

In one embodiment, nucleic acid sensor molecules are employed to follow the fate of a drug or its metabolic by-products. In this embodiment, nucleic acid sensor molecules are generated to the drug and its metabolites. The drug is administered to the test animal either subcutaneously, intraperitoneally or by gavage. Subsequent to administration, the blood plasma or disease tissue is removed and its contents are screened for the remaining drug by Liquid chromatography (LC) or LC-mass spectrometry. Drug exposure is then determined as a function of time, dose and method of administration and is reported in values of half-life, bioavailability, AUC and Cmax. Metabolic products of a drug lead can be similarly followed.

Nucleic acid sensor molecules generated against enzymes or proteins known to those skilled in the art to be involved in drug metabolism (P45 0 enzymes, multi-drug transporter) can be used to follow the effect of a drug on xenobiotic or topological response to drug treatment.

Step 9. Optimization of the Drug Lead

In one embodiment, drug-lead potency, specificity, and/or in vivo 10 activity, and pharmacokinetic, or toxological properties are optimized by an iterative repetition repetition of any or all of steps 1-7. In one embodiment, steps 1-7 are repeated until the desired potency, selectivity and in vivo activity and pharmaco-kinetic, or toxological properties of a candidate drug are obtained.

Step 10. Clinical Trials

In one embodiment, nucleic acid sensor molecules are used in clinical trials to determine the fate of a drug in human or animal models, or used to follow the effect of drug treatment on a target or molecular pathway of choice, as described above. In one embodiment, the nucleic acid sensor molecules, in a solid phase assay (e.g., ELISA format), a solution phase assay, or in a pathway profiling biosensor array format, are used to assess the in vivo biological activity of a drug being tested using lysed cell samples as described above.

In another embodiment, the appropriate pathway profiling biosensor is used in vivo, to monitor the effects of the compound on the patient, for example, by providing the biosensor in communication with a fiber optic probe inserted into the patient, or ex vivo, monitoring optical signals in a cell using a microscope based detection system. In another embodiment, an in vivo assay is done by introducing a nucleic acid sensor molecule which retains its catalytic activity into a physiological system (e.g., by injection at a target site in the body, through liposome carriers, and other means of administration routinely used in the art), obtaining cells from the physiological system and detecting the effect of the compound on the catalytic activity of the nucleic acid sensor molecule (e.g., by evaluating the sequence of the nucleic acid sensor molecule) as a means of determining the level, structure, or activity of a drug target, and relating the level, structure, or activity or the target molecules to the efficacy of the drug.

Step 11. Optimization of the Drug Lead

In one embodiment, any or all of steps 1-10 are repeated to further optimize the properties of the candidate drug.

Step 12. Diagnostic Applications

In one embodiment, individuals who would be suitable for treatment with the candidate drugs identified steps 1-11, are identified using nucleic acid sensor molecules in the diagnostic assays discussed previously.

Step 13. Chemical Genomics

In one embodiment, nucleic acid sensor molecules are used in chemical genomic assays in which a drug or plurality of drug leads, with known or unknown physiological effects, and with unknown targets, are contacted with a physiological system and the site of action of the drug or plurality of drugs is determined using a plurality of the profiling biosensors or pathway profiling biosensors described previously. Drug optimization then occurs as in steps1-11.

5. Use of Target Activated Biosensors in Target Molecule Separation

In addition to, or instead of, their use in detection methods, and drug discovery methods, the target activated biosensors according to the invention can also be used to retrieve the target molecules to which they specifically bind. Additional embodiments exploiting the binding capacity of the biosensors disclosed are contemplated and encompassed within the scope.

Reagents for Generating and Using Nucleic Acid Sensor Molecules

In one embodiment, reagents are provided for generating and using nucleic acid sensor molecules. In one embodiment, a kit is provided comprising standardized reagents for making and/or using the nucleic acid sensor molecules according to the invention. In one embodiment, the kit comprises at least a first nucleic acid sensor molecule whose optical properties change upon binding of a target molecule. In another embodiment, the kit additionally comprises any of: a control target molecule, a second nucleic acid sensor molecule which binds to a different target molecule, suitable buffers, printed instructions, and combinations thereof. In a further embodiment, a nucleic acid sensor molecule is provided with reagents for attaching a label and/or quencher or with reagents for attaching charge transfer molecules to the nucleic acid sensor molecule, which can sensitize the optical properties of the nucleic acid molecule to the presence of a target molecule.

In another embodiment, a composition is provided comprising a target molecule and a nucleic acid sensor molecule. The composition provides a reference against which to compare modified nucleic acid sensor molecules which bind to the same target, in order to select those with higher affinities for the target. In a further embodiment, sets of complexes are provided. In still a firther embodiment, a set of pathway target molecules and sensor molecules are provided. In another embodiment, a set of profiling target molecules and nucleic acid sensor molecules are provided. In still a further embodiment, solid supports are provided for isolation of target molecules from nucleic acid sensor molecules.

In yet another embodiment, a computer program product is provided comprising stored data relating to optical signals generated by profiling and or pathway target molecules. In another embodiment, a means to compare this data to other optical signals is provided. In a further embodiment, the memory comprises data relating to patient information or chemical structure information relating to either target molecules or nucleic acid sensor molecules.

The nucleic acid sensor molecules and target activated biosensors according to the invention are amenable for use with high throughput screening systems and methods and the use of the nucleic acid sensor molecules and target activated biosensors in these systems and methods is encompassed within the scope. In one embodiment, the system is a robotic workstation, comprising, at least one of an: arrayer, microplate or microarray feeders, stackers, washers, and dispensers, an optical system, a carousel, a conveyer for conveying microplates or microarrays from one part of the system to another (in a vertical or horizontal direction), a shaker system or other mixing system, a temperature control system, a synthesizer, a solid phase extraction system, and sample concentrators. Components of the robotic workstation can be part of a single integrated system or can be provided separately for use at any stage of the drug optimization process according to the invention. In a further embodiment, the system comprises a processor connectable to the network which comprises or can access applications comprising stored data relating to profiling information obtained using nucleic acid sensor molecules according to the invention, and/or statistical applications, applications for performing structure/activity analysis of target molecules and nucleic acid sensor molecules, applications for performing nucleic acid sequence alignment and simultaneous structure superposition of proteins (e.g., MOE-Align'™), applications for predicting binding conformations of molecules to receptor structures, and applications for controlling the processing functions of the robotic workstations.

The invention is further illustrated in the following non-limiting examples.

### EXAMPLE 1

### Preparation of an Array of Immobilized Effector Oligonucleotides

The following protocol describes a method for preparing an array of immobilized effector oligonucleotides with terminal amine groups to a solid substrate derivatized with aldehyde groups. The resulting array can then be used to spatially address (i.e., the sequence of nucleotides for each effector oligo can be synthesized as a cognate to a ribozyme sensor specific for a particular target molecule) and immobilize the ribozyme sensor molecules prior to use in a solid-phase assay (see, e.g., Zammatte et al., 2000):

Protocol for attachment of oligonucleotides to aldehyde substrate (www.arrayit.com):

1. print discrete spots of solution containing oligonucleotides with amine-reactive terminal groups or linkers with terminal amine groups using microarraying pins, pipette, etc.

2. allow substrate to dry for 12 hrs. at room temperature and <30% relative humidity.

3. rinse substrate 2 times in dH20 with 0.2% SDS for 2 min. with vigorous agitation at room temperature.

4. rinse substrate 1 time in dH20 for 2 min. with vigorous agitation at room temperature.

5. transfer substrate to boiling (100 degrees C.) dH20 for 3 min. to denature DNA.

6. dry substrate by centrifugation at 500×g for 1 min.

7. treat substrate in 0.1 M NaBH4 in phosphate buffered saline (PBS, pH 7) for 5 min. with mild agitation at room temperature.

8. rinse substrate 2 times in dH20 with 0.2% SDS for 1 min. with vigorous agitation at room temperature.

9. rinse substrate 1 times in dH20 for 2 min. with vigorous agitation at room temperature.

10. transfer substrate to boiling (100 degrees C.) dH20 for 10 sec. to denature DNA.

11. dry substrate by centrifugation at 500×g for 1 min.

12. store oligonucleotide-bound substrate at 4 degrees C. prior to hybridization.

If desired, the nucleic acid sensor molecules can be allosteric ribozymes which ligate or self-cleave a substrate in the presence of a target molecule (see FIGS. 2A and B for the ligater, FIG. 5 for the cleaver). Here, the ribozymes are bound to a solid substrate directly via their 3′ termini. The attachment is accomplished by oxidation (using, e.g., Na periodate) of the 3′ vicinal diol of the ribozyme to an aldehyde group. This aldehyde group will react with a hydrazide group to form a hydrazone bond. The hydrazone bond is quite stable to hydrolysis, etc., but can be further reduced (for example, by treatment with NaBH4 or NaCNBH3). The use of adipic acid dihydrazide (ADH, a bifunctional linker) to derivatize an aldehyde surface results in a hydrazide-derivatized surface which provides a linker between the substrate surface and point of biomolecular attachment of approximately 10 atoms (see Ruhn et al., 1994; O'Shaughnessy, 1990; Roberston et al., 1972, Schluep et al., 1999; Chan et al., 1998). Preparation of a hydrazide-terminated surface via ADH treatment can be accomplished by treating an aldehyde-derivatized substrate according to the following protocol:

Protocol for ADH treatment of aldehyde substrate:

1. to 50 mL of 0.1 M phosphate buffer (pH 5), add 100-fold excess of adipic acid dihydrazide (ADH) relative to concentration of aldehyde groups on substrate surface.

2. place substrate in a 50 mL tube containing the ADH in phosphate buffer and shake mixture for 2 hrs.

3. remove the substrate and wash 4 times with 0.1 M phosphate buffer (pH 7).

4. reduce free aldehyde groups on substrate surface by placing substrate in a 50 mL tube containing a 25-fold excess of NaBH4 or NaCNBH3 in 0.1 M phosphate buffer.

5. shake the mixture for 90 min.

6. wash 4 times with 0.1 M phosphate buffer (pH 7).

7. store ADH-treated substrates in 0.1 M phosphate buffer (pH 7) at 4 degrees C.

Preparation of the nucleic acid molecules for specific coupling to the ADH-terminated surface via their 3′ termini can be accomplished according to the following protocol (see, Proudnikov et al., 1996; Wu et al., 1996):

Protocol for Periodate oxidation of RNA:

1. dissolve up to 20 micrograms of RNA in 5 microliters of H20 at 20 degrees C.

2. add 1 microliter of 0.1 M NaIO4 (˜20-fold excess relative to RNA).

3. incubate for 30 min. in a light-tight tube or enclosure.

4. add 1 microliter of 0.2 M Na sulphite (˜2-fold excess relative to NaIO4) to stop reaction.

5. incubate for 30 min. at room temperature. ethanol precipitate or use spin-separation column to recover oxidized RNA.

### EXAMPLE 2

### Selection for a Riboreporter Selective for the Estrogen Receptor LBD

A Riboreporter specific for the estrogen receptor ligand binding domain (LBD) is obtained by identifying candidate nucleic acids that bind to an estrogen receptor LBD.

The full length gene for the estrogen receptor is known. One source of the full-length estrogen receptor clone is Acc. No. M12674 (see also Greene et al., Science 231:1150-54, 1986). The clone includes a 2092 nucleotide mRNA with the following sequence:

| | |
|---|---|
| 1 | (SEQ ID NO:9) |
| | gaattccaaa attgtgatgt ttcttgtatt tttgatgaag |
| | gagaaatact gtaatgatca |
| 61 | ctgtttacac tatgtacact ttaggccagc cctttgtagc |
| | gttatacaaa ctgaaagcac |
| 121 | accggacccg caggctcccg gggcagggcc ggggccagag |
| | ctcgcgtgtc ggcgggacat |
| 181 | gcgctgcgtc gcctctaacc tcgggctgtg ctctttttcc |
| | aggtggcccg ccggtttctg |
| 241 | agccttctgc cctgcgggga cacggtctgc accctgcccg |
| | cggccacgga ccatgaccat |
| 301 | gaccctccac accaaagcat ctgggatggc cctactgcat |
| | cagatccaag ggaacgagct |
| 361 | ggagcccctg aaccgtccgc agctcaagat ccccctggag |
| | cggcccctgg gcgaggtgta |
| 421 | cctggacagc agcaagcccg ccgtgtacaa ctaccccgag |
| | ggcgccgcct acgagttcaa |
| 481 | cgccgcggcc gccgccaacg cgcaggtcta cggtcagacc |
| | ggcctcccct acggccccgg |
| 541 | gtctgaggct gcggcgttcg gctccaacgg cctggggggt |
| | ttccccccac tcaacagcgt |
| 601 | gtctccgagc ccgctgatgc tactgcaccc gccgccgcag |
| | ctgtcgcctt tcctgcagcc |
| 661 | ccacggccag caggtgccct actacctgga gaacgagccc |
| | agcggctaca cggtgcgcga |
| 721 | ggccggcccg ccggcattct acaggccaaa ttcagataat |
| | cgacgccagg gtggcagaga |
| 781 | aagattggcc agtaccaatg acaagggaag tatggctatg |
| | gaatctgcca aggagactcg |
| 841 | ctactgtgca gtgtgcaatg actatgcttc aggctaccat |
| | tatggagtct ggtcctgtga |
| 901 | gggctgcaag gccttcttca agagaagtat tcaaggacat |
| | aacgactata tgtgtccagc |
| 961 | caccaaccag tgcaccattg ataaaaacag gaggaagagc |
| | tgccaggcct gccggctccg |
| 1021 | caaatgctac gaagtgggaa tgatgaaagg tgggatacga |
| | aaagaccgaa gaggagggag |
| 1081 | aatgttgaaa cacaagcgcc agagagatga tggggagggc |
| | aggggtgaag tggggtctgc |
| 1141 | tggagacatg agagctgcca acctttggcc aagcccgctc |
| | atgatcaaac gctctaagaa |
| 1201 | gaacagcctg gccttgtccc tgacggccga ccagatggtc |
| | agtgccttgt tggatgctga |
| 1261 | gccccccata ctctattccg agtatgatcc taccagaccc |
| | ttcagtgaag cttcgatgat |
| 1321 | gggcttactg accaacctgg cagacaggga gctggttcac |
| | atgatcaact gggcgaagag |
| 1381 | ggtgccaggc tttgtggatt tgaccctcca tgatcaggtc |
| | caccttatag aatgtgcctg |
| 1441 | gctagagatc ctgatgattg gtctcgtctg gcgctccatg |
| | gagcacccag tgaagctact |
| 1501 | gtttgctcct aacttgctct tggacaggaa ccagggaaaa |
| | tgtgtagagg gcatggtgga |
| 1561 | gatcttcgac atgctgctgg ctacatcatc tcggttccgc |
| | atgatgaatc tgcagggaga |
| 1621 | ggagtttgtg tgcctcaaat ctattatttt gcttaattct |
| | ggagtgtaca catttctgtc |
| 1681 | cagcaccctg aagtctctgg aagagaagga ccatatccac |
| | cgagtcctgg acaagatcac |
| 1741 | agacactttg atccacctga tggccaaggc aggcctgacc |
| | ctgcagcagc agcaccagcg |
| 1801 | gctggcccag ctcctcctca tcctctccca catcaggcac |
| | atgagtaaca aaggcatgga |
| 1861 | gcatctgtac agcatgaagt gcaagaacgt ggtgcccctc |
| | tatgacctgc tgctggagat |
| 1921 | gctggacgcc caccgcctac atgcgcccac tagccgtgga |
| | ggggcatccg tggaggagac |
| 1981 | ggaccaaagc cacttggcca ctgcgggctc tacttcatcg |
| | cattccttgc aaaagtatta |
| 2041 | catcacgggg gaggcagagg gtttccctgc cacagtctga |
| | gagctccctg gc |

The polynucleotide encodes a polypeptide with the following amino acid sequence:

(SEQ ID NO:10)
MTMTLHTKASGMALLHQIQGNELEPLNRPQLKIPLERPLGEVYLDSSKPA
VYNYPEGAAYEFNAAAAANAQVYGQTGLPYGPGSEAAAFGSNGLGGFPPL
NSVSPSPLMLLHPPPQLSPFLQPHGQQVPYYLENEPSGYTVREAGPPAFY
RPNSDNRRQGGRERLASTNDKGSMAMESAKETRYCAVCNDYASGYHYGVW
SCEGCKAFFKRSIQGHNDYMCPATNQCTIDKNRRKSCQACRLRKCYEVGM
MKGGIRKDRRGGRMLKHKRQRDDGEGRGEVGSAGDMRAANLWPSPLMIKR
SKKNSLALSLTADQMVSALLDAEPPILYSEYDPTRPFSEASMMGLLTNLA
DRELVHMINWAKRVPGFVDLTLHDQVHLLECAWLEILMIGLVWRSMEHPV
KLLFAPNLLLDRNQGKCVEGMVEIFDMLLATSSRFRMMNLQGEEFVCLKS
IILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRLH
APTSRGGASVEETDQSHLATAGSTSSHSLQKYYITGEAEGFPATV

The gene encoding either full length ER or the ligand binding domain is cloned and expressed in BL21 (DE3)-pLysS E. coli cells [Shiau A K, Barstad D, Loria P M, Cheng L, Kushner P J, Agard D A, Greene G L. Cell. Dec. 23, 1998;95(7):927-37.]. Human ERalpha LBD (residues 297-554) are purified from estradiol-sepharose column by published procedures [Shiau A K, Barstad D, Loria P M, Cheng L, Kushner P J, Agard D A, Greene G L. Cell. Dec. 23, 1998;95(7):927-37.]. ER-LBD complexes are then formed upon 1:1 complexing with estrodiol, or with tamoxifen.

### EXAMPLE 3

### Selection of Riboreporters which are Activated by ER-LBD not Bound to Ligand

A library of up to 10¹⁷ variants of in vitro synthesized ribozymes is allowed to react with purified apo-ER-LBD at a final concentration of 1 uM. Selection of allosterically activated ribozymes is carried out by procedures outlined in prior examples.

### EXAMPLE 4

### Selection of Riboreporters which are Activated by the ER-LBD-Estradiol Complex.

A library of up to 10¹⁷ variants of in vitro synthesized ribozymes is allowed to react with purified ER-LBD-estradiol at a final complex concentration of 1 uM. Selection of allosterically activated ribozymes is carried out by procedures outlined in prior examples.

### EXAMPLE 5

### Selection of Riboreporters which are Activated by the ER-LBD-tamoxifen Complex.

A library of up to 10¹⁷ variants of in vitro synthesized ribozymes is allowed to react with purified ER-LBD-estradiol at a final complex concentration of 1 uM. Selection of allosterically activated ribozymes is carried out by procedures outlined in prior examples.

### EXAMPLE 6

### Selection for a Library of Riboreporters which Signal the Presence of all Known Nuclear Hormone Receptor LBDs:

N-terminally GST-tagged or N-/C-terminally His-tagged ligand binding domains, defined on the basis of structural homology are cloned and expressed in BL21 (DE3)-pLysS E. coli cells, or are cloned and expressed in standard baculovirus expression systems. References for the sequences are provided below:

| Symbol | Description | Ligand |
|---|---|---|
| A1B3 | nuclear receptor coactivator RAP250; peroxisome | thyroid hormone |
| | proliferator-activated receptor interacting protein; | |
| | thyroid hormone receptor binding protein | |
| AR | androgen receptor (dihydrotestosterone receptor; | dihydroxytestosteron |
| | testicular feminization; spinal and bulbar muscular | |
| | atrophy; Kennedy disease) | |
| C1D | nuclear DNA-binding protein | |
| ESR1 | estrogen receptor 1 | estrogen |
| ESR2 | estrogen receptor 2 (ER beta) | estrogen |
| ESRRA | estrogen-related receptor alpha | estrogen and TFIIB |
| ESRRB | estrogen-related receptor beta | estrogen and TFIIB |
| ESRRG | estrogen-related receptor gamma | estrogen and TFIIB |
| HNF4A | hepatocyte nuclear factor 4, alpha | |
| HNF4G | hepatocyte nuclear factor 4, gamma | |
| | similar to retinoid X receptor, alpha (H. sapiens) | |
| | similar to nuclear receptor subfamily 1, group D, | |
| | member 1 (H. sapiens) | |
| NCOA1 | nuclear receptor coactivator 1 | Binds to steroid hormone |
| | | receptors |
| NCOR1 | nuclear receptor co-repressor 1 | Yhyroid hormone receptor |
| | | without TH |
| NCOR2 | nuclear receptor co-repressor 2 | RXR without retinoic acid and |
| | | THR without TH |
| NR0B1 | nuclear receptor subfamily 0, group B, member 1 | |
| NR0B2 | nuclear receptor subfamily 0, group B, member 2 | |
| NR1D1 | nuclear receptor subfamily 1, group D, member 1 | |
| NR1H2 | nuclear receptor subfamily 1, group H, member 2 | |
| NR1H3 | nuclear receptor subfamily 1, group H, member 3 | Intertacts with RXR |
| NR1H4 | nuclear receptor subfamily 1, group H, member 4 | Bile acid, farnesol, or |
| | | chenodoxycholic acid |
| NR1I2 | nuclear receptor subfamily 1, group I, member 2 | pregnane |
| NR1I3 | nuclear receptor subfamily 1, group I, member 3 | androstane S |
| NR2C1 | nuclear receptor subfamily 2, group C, member 1 | |
| NR2C2 | nuclear receptor subfamily 2, group C, member 2 | |
| NR2E1 | nuclear receptor subfamily 2, group E, member 1 | |
| NR2E3 | nuclear receptor subfamily 2, group E, member 3 | |
| NR2F1 | nuclear receptor subfamily 2, group F, member 1 | |
| NR2F2 | nuclear receptor subfamily 2, group F, member 2 | |
| NR2F6 | nuclear receptor subfamily 2, group F, member 6 | Thyroid hormone |
| NR3C1 | nuclear receptor subfamily 3, group C, member 1 | glutocorticoid receptor, |
| | | cortisol, corticosterone |
| NR3C2 | nuclear receptor subfamily 3, group C, member 2 | Aldosterone |
| NR4A1 | nuclear receptor subfamily 4, group A, member 1 | |
| NR4A2 | nuclear receptor subfamily 4, group A, member 2 | |
| NR4A3 | nuclear receptor subfamily 4, group A, member 3 | |
| NR5A1 | nuclear receptor subfamily 5, group A, member 1 | |
| NR5A2 | nuclear receptor subfamily 5, group A, member 2 | |
| NR6A1 | nuclear receptor subfamily 6, group A, member 1 | |
| PAX8 | paired box gene 8 | |
| PGR | progesterone receptor | progesterone |
| PPARA | peroxisome proliferative activated receptor, alpha | nafenopin, clofibrate, |
| | | WY14643 |
| PPARBP | PPAR binding protein | binds to PPAR gamma |
| PPARD | peroxisome proliferative activated receptor, delta | WY1463 |
| PPARG | peroxisome proliferative activated receptor, gamma | 9-HODE, 13-HODE |
| PTHR1 | parathyroid hormone receptor 1 | parathyroid hormone |
| RARA | retinoic acid receptor, alpha | retinoic acid |
| RARB | retinoic acid receptor, beta | retinoic acid |
| RARG | retinoic acid receptor, gamma | retinoic acid |
| RORA | RAR-related orphan receptor A | |
| RORB | RAR-related orphan receptor B | |
| RORC | RAR-related orphan receptor C | |
| RXRA | retinoid X receptor, alpha | 9-cis retonoic acid, |
| | | complexes with activated |
| | | VDR and THR |
| RXRB | retinoid X receptor, beta | 9-cis retonoic acid, |
| | | complexes with activated |
| | | VDR and THR |
| RXRG | retinoid X receptor, gamma | 9-cis retonoic acid, |
| | | complexes with activated |
| | | VDR and THR |
| SMAP | thyroid hormone receptor coactivating protein | activated THR |
| THRA | thyroid hormone receptor, alpha (avian | thyroid hormone |
| | erythroblastic leukemia viral (v-erb-a) oncogene | |
| | homolog) | |
| THRB | thyroid hormone receptor, beta (avian erythroblastic | thyroid hormone |
| | leukemia viral (v-erb-a) oncogene homolog 2) | |
| TNRC11 | trinucleotide repeat containing 11 (THR-associated | activated THR |
| | protein, 230 kDa subunit) | |
| TRAP150 | thyroid hormone receptor-associated protein, 150 | activated THR |
| | kDa subunit | |
| TRAP240 | thyroid hormone receptor-associated protein, 240 | activated THR |
| | kDa subunit | |
| TRAP95 | thyroid hormone receptor-associated protein, 95-kD | activated THR |
| | subunit | |
| TRIP13 | thyroid hormone receptor interactor 13 | inactive THR |
| VDR | vitamin D (1,25-dihydroxyvitamin D3) receptor | vitamin D |

Human NHR LBDs (homologous to ER-alpha residues including the region aa297-554) are purified from GSH-sepharose or nickel affinity columns by published procedures available from the manufacturers. LBDs are produced in a either a parallel or serial fashion and the purified proteins are stored as in buffer containing 50 mM TrisHCl, 1 mM EDTA, 1 mM DTT and 50-250 Na Cl/SCN salt, pH 7 to pH 8.5, 10% glycerol or other stabilizing agent. Protein sequence and MW is verified by standard mass spectrometry.

1] Selection of Riboreporters which are activated by un-liganded 63 NHR-LBD: A library of up to 10 E+17 variants of in vitro synthesized ribozymes is allowed to react with purified apo-NHR-LBDs at a final concentration of 1 uM LBD. Selection of allosterically activated ribozymes is carried out by procedures outlined in prior examples. Selections are carried out in parallel fashion or also can be carried out in mixed pools of anywhere from 5-10 HNR LBDs. In the final rounds of riboreporter selection, the RNA pools may separated into aliquots which may then be used to carry out in vitro selection against single NHR LBD proteins to yield unique riboreporters selective for multiple NHR LBDs.

2. Selection of Riboreporters which are activated by ligand bound forms of NHR-LBDs. Ligands for multiple NHR are known. These ligands are provided below.

| | | |
|---|---|---|
| AIB3 | nuclear receptor | thyroid hormone |
| | coactivator RAP250; | |
| | peroxisome | |
| | proliferator-activated | |
| | receptor interacting | |
| | protein; thyroid | |
| | hormone receptor | |
| | binding protein | |
| AR | androgen receptor | dihydroxytestosteron |
| | (dihydrotestosterone | |
| | receptor; testicular | |
| | feminization; spinal | |
| | and bulbar muscular | |
| | atrophy; Kennedy | |
| | disease) | |
| C1D | nuclear DNA-binding | |
| | protein | |
| ESR1 | estrogen receptor 1 | estrogen |
| ESR2 | estrogen receptor 2 | |
| | (ER beta) | estrogen |
| ESRRA | estrogen-related | estrogen and TFIIB |
| | receptor alpha | |
| ESRRB | estrogen-related | estrogen and TFIIB |
| | receptor beta | |
| ESRRG | estrogen-related | estrogen and TFIIB |
| | receptor gamma | |
| HNF4A | hepatocyte nuclear | |
| | factor 4, alpha | |
| HNF4G | hepatocyte nuclear | |
| | factor 4, gamma | |
| | similar to retinoid X | |
| | receptor, alpha (H. | |
| | sapiens) | |
| | similar to nuclear | |
| | receptor subfamily 1, | |
| | group D, member 1 | |
| | (H. sapiens) | |
| NCOA1 | nuclear receptor | Binds to steroid |
| | coactivator 1 | hormone receptors |
| NCOR1 | nuclear receptor co- | thyroid hormone receptor |
| | repressor 1 | without TH |
| NCOR2 | nuclear receptor co- | RXR without retinoic acid |
| | repressor 2 | and THR without TH |
| NR0B1 | nuclear receptor | |
| | subfamily 0, group B, | |
| | member 1 | |
| NR0B2 | nuclear receptor | |
| | subfamily 0, group B, | |
| | member 2 | |
| NR1D1 | nuclear receptor | |
| | subfamily 1, group D, | |
| | member 1 | |
| NR1H2 | nuclear receptor | |
| | subfamily 1, group H, | |
| | member 2 | |
| NR1H3 | nuclear receptor | Intertacts with RXR |
| | subfamily 1, group H, | |
| | member 3 | |
| NR1H4 | nuclear receptor | Bile acid, famesol, or |
| | subfamily 1, group H, | chenodoxycholic acid |
| | member 4 | |
| NR1I2 | nuclear receptor | pregnane |
| | subfamily 1, group I, | |
| | member 2 | |
| NR1I3 | nuclear receptor | androstane S |
| | subfamily 1, group I, | |
| | member 3 | |
| NR2C1 | nuclear receptor | |
| | subfamily 2, group C, | |
| | member 1 | |
| NR2C2 | nuclear receptor | |
| | subfamily 2, group C, | |
| | member 2 | |
| NR2E1 | nuclear receptor | |
| | subfamily 2, group E, | |
| | member 1 | |
| NR2E3 | nuclear receptor | |
| | subfamily 2, group E, | |
| | member 3 | |
| NR2F1 | nuclear receptor | |
| | subfamily 2, group F, | |
| | member 1 | |
| NR2F2 | nuclear receptor | |
| | subfamily 2, group F, | |
| | member 2 | |
| NR2F6 | nuclear receptor | Thyroid hormone |
| | subfamily 2, group F, | |
| | member 6 | |
| NR3C1 | nuclear receptor | glutocorticoid receptor, |
| | subfamily 3, group C, | cortisol, corticosterone |
| | member 1 | |
| NR3C2 | nuclear receptor | aldosterone |
| | subfamily 3, group C, | |
| | member 2 | |
| NR4A1 | nuclear receptor | |
| | subfamily 4, group A, | |
| | member 1 | |
| NR4A2 | nuclear receptor | |
| | subfamily 4, group A, | |
| | member 2 | |
| NR4A3 | nuclear receptor | |
| | subfamily 4, group A, | |
| | member 3 | |
| NR5A1 | nuclear receptor | |
| | subfamily 5, group A, | |
| | member 1 | |
| NR5A2 | nuclear receptor | |
| | subfamily 5, group A, | |
| | member 2 | |
| NR6A1 | nuclear receptor | |
| | subfamily 6, group A, | |
| | member 1 | |
| PAX8 | paired box gene 8 | |
| PGR | progesterone receptor | progesterone |
| PPARA | peroxisome | nafenopin, clofibrate, WY14643 |
| | proliferative activated | |
| | receptor, alpha | |
| PPARBP | PPAR binding protein | binds to PPAR gamma |
| PPARD | peroxisome | WY14643 |
| | proliferative activated | |
| | receptor, delta | |
| PPARG | peroxisome | 9-HODE, 13-HODE |
| | proliferative activated | |
| | receptor, gamma | |
| PTHR1 | parathyroid hormone | parathyroid hormone |
| | receptor 1 | |
| RARA | retinoic acid receptor, | retinoic acid |
| | alpha | |
| RARB | retinoic acid receptor, | retinoic acid |
| | beta | |
| RARG | retinoic acid receptor, | retinoic acid |
| | gamma | |
| RORA | RAR-related orphan | |
| | receptor A | |
| RORB | RAR-related orphan | |
| | receptor B | |
| RORC | BAR-related orphan | |
| | receptor C | |
| RXRA | retinoid X receptor, | 9-cis retonoic acid, |
| | alpha | complexes with activated VDR |
| | | and THR |
| RXRB | retinoid X receptor, | 9-cis retonoic acid, |
| | beta | complexes with activated VDR |
| | | and THR |
| RXRG | retinoid X receptor, | 9-cis retonoic acid, |
| | gamma | complexes with activated VDR |
| | | and THR |
| SMAP | thyroid hormone | activated THR |
| | receptor coactivating | |
| | protein | |
| THRA | thyroid hormone | thyroid hormone |
| | receptor, alpha (avian | |
| | erythroblastic | |
| | leukemia viral (v-erb- | |
| | a) oncogene | |
| | homolog) | |
| THRB | thyroid hormone | thyroid hormone |
| | receptor, beta (avian | |
| | erythroblastic | |
| | leukemia viral (v-erb- | |
| | a) oncogene homolog | |
| | 2) | |
| TNRC11 | trinucleotide repeat | activated THR |
| | containing 11 (THR- | |
| | associated protein, | |
| | 230 kDa subunit) | |
| TRAP150 | thyroid hormone | activated THR |
| | receptor-associated | |
| | protein, 150 kDa | |
| | subunit | |
| TRAP240 | thyroid hormone | activated THR |
| | receptor-associated | |
| | protein, 240 kDa | |
| | subunit | |
| TRAP95 | thyroid hormone | activated THR |
| | receptor-associated | |
| | protein, 95-kD | |
| | subunit | |
| TRIP13 | thyroid hormone | inactive THR |
| | receptor interactor 13 | |
| VDR | vitamin D (1,25- | vitamin D |
| | dihydroxyvitamin D3) | |
| | receptor | |

Stable complexes of each NHR LBD are formed with from 1-10 equivalents of ligand. Selection of Riboreporters which are activated by the NHR-LBD-ligand complex. A library of up to 10¹⁷ variants of in vitro synthesized ribozymes is allowed to react with purified ER-LBD-estradiol at a final complex concentration of 1 uM. Selection of allosterically activated ribozymes is carried out by procedures outlined in prior examples. Selections are carried out in parallel fashion or also can be carried out in mixed pools of anywhere from 5-10 HNR LBD-ligand complexes. In the final rounds of riboreporter selection, the RNA pools may separated into aliquots which may then be used to carry out in vitro selection against single NHR LBD-ligand complex to yield unique riboreporters selective for all NHR LBDs.

Thus, the invention provides an in vitro selection protocol against purified LBDs for each known NHR. In vitro selections can be carried out with less than 1 mg of the purified forms of the LBDs. In addition the selection of riboreporters can be done in vitro with mixed pools of LBD and subsequently deconvoluted after selection is complete. Alternatively, the final selection can be carried out with fractionally purified extracts containing a slight excess of recombinant LBD. In one embodiment the LBD is expressed in E.coli or insect cell line or mammalian cell lines. In another embodiment, the selection is carried out in cell free lysates in which the LBD is expressed in an in vitro transcription-translation procedure such as is described in the literature or can be purchased using common reagents from Roche or Promega. In another embodiment, the fractionated or purified LBDs are combined with known ligands, agonist, antagonists or partial agonist/antagonists to form stable complexes, and these complexes are then used for in vitro selection of riboreporters. For this selection, we will utilize the entire protein in complex with ligand or a peptide component derived from the NHR. Upon interaction of the riboreporter with the NHR, a signal will be generated detectable to an external monitoring device. In this manner, the activation state of any or all NHRs can be monitored in vivo or in vitro.

### EXAMPLE 7

### Selection for a Riboreporter Selective for the Beta-2 Adrenergic Receptor

The full-length gene for the Beta-2 adrenergic receptor is described in Emorine et al., Proc. Natl. Acad. Sci. USA 84:6995-99, 1987 and available at Acc. No. AAA88017. The nucleic acid sequence is set forth below:

(SEQ ID NO:11)
GCACCGCGAGCCCCTAGCACCCGACAAGCTGAGTGTGCAGGACGAGTCCC
CACCACACCCACACCACAGCCGCTGAATGAGGCTTCCAGGCGTCCGCTCG
CGGCCCGCAGAGCCCCGCCGTGGGTCCGCCCGCTGAGGCGCCCCCAGCCA
GTGCGCTTACCTGCCAGACTGCGCGCCATGGGGCAACCCGGGAACGGCAG
CGCCTTCTTGCTGGCACCCAATGGAAGCCATGCGCCGGACCACGACGTCA
CGCAGGAAAGGGACGAGGTGTGGGTGGTGGGCATGGGCATCGTCATGTCT
CTCATCGTCCTGGCCATCGTGTTTGGCAATGTGCTGGTCATCACAGCCAT
TGCCAAGTTCGAGCGTCTGCAGACGGTCACCAACTACTTCATCACTTCAC
TGGCCTGTGCTGATCTGGTCATGGGCCTGGCAGTGGTGCCCTTTGGGGCC
GCCCATATTCTTATGAAAATGTGGACTTTTGGCAACTTCTGGTGCGAGTT
TTGGACTTCCATTGATGTGCTGTGCGTCACGGCCAGCATTGAGACCCTGT
GCGTGATCGCAGTGGATCGCTACTTTGCCATTACTTCACCTTTCAAGTAC
CAGAGCCTGCTGACCAAGAATAAGGCCCGGGTGATCATTCTGATGGTGTG
GATTGTGTCAGGCCTTACCTCCTTCTTGCCCATTCAGATGCACTGGTACC
GGGCCACCCACCAGGAAGCCATCAACTGCTATGCCAATGAGACCTGCTGT
GACTTCTTCACGAACCAAGCCTATGCCATTGCCTCTTCCATCGTGTCCTT
CTACGTTCCCCTGGTGATCATGGTCTTCGTCTACTCCAGGGTCTTTCAGG
AGGCCAAAAGGCAGCTCCAGAAGATTGACAAATCTGAGGGCCGCTTCCAT
GTCCAGAACCTTAGCCAGGTGGAGCAGGATGGGCGGACGGGGCATGGACT
CCGCAGATCTTCCAAGTTCTGCTTGAAGGAGCACAAAGCCCTCAAGACGT
TAGGCATCATCATGGGCACTTTCACCCTCTGCTGGCTGCCCTTCTTCATC
GTTAACATTGTGCATGTGATCCAGGATAACCTCATCCGTAAGGAAGTTTA
CATCCTCCTAAATTGGATAGGCTATGTCAATTCTGGTTTCAATCCCCTTA
TCTACTGCCGGAGCCCAGATTTCAGGATTGCCTTCCAGGAGCTTCTGTGC
CTGCGCAGGTCTTCTTTGAAGGCCTATGGGAATGGCTACTCCAGCAACGG
CAACACAGGGGAGCAGAGTGGATATCACGTGGAACAGGAGAAAGAAAATA
AACTGCTGTGTGAAGACCTCCCAGGCACGGAAGACTTTGTGGGCCATCAA
GGTACTGTGCCTAGCGATAACATTGATTCACAAGGGAGGAATTGTAGTAC
AAATGACTCACTGCTGTAAAGCAGTTTTTCTACTTTTAAAGACCCCCCCC
CCCCCAACAGAACACTAAACAGACTATTTAACTTGAGGGTAATAAACTTA
GAATAAAATTGTAAAAATTGTATAGAGATATGCAGAAGGAAGGGCATCCT
TCTGCCTTTTTTATTTTTTTAAGCTGTAAAAAGAGAGAAAACTTATTTGA
GTGATTATTTGTTATTTGTACAGTTCAGTTCCTCTTTGCATGGAATTTGT
AAGTTTATGTCTAAAGAGCTTTAGTCCTAGAGGACCTGAGTCTGCTATAT
TTTCATGACTTTTCCATGTATCTACCTCACTATTCAAGTATTAGGGGTAA
TATATTGCTGGTAATTTGTATCTGAAGGAGATTTTCCTTCCTACACCCTT
GGACTTGAGGATTTTGAGTATCTCGGACCTTTCAGCTGTGAACATGGACT
CTTCCCCCACTCCTCTTATTTGCTCACACGGGGTATTTTAGGCAGGGATT
TGAGGAGCAGCTTCAGTTGTTTTCCCGAGCAAAGGTCTAAAGTTTACAGT
AAATAAAATGTTTGACCATG

The amino acid sequence of the polypeptide encoded by the nucleic acid sequence is set forth below:

| | |
|---|---|
| | (SEQ ID NO:12) |
| 1 | mgqpgngsaf llapngshap dhdvtqqrde vwvvgmgivm |
| | slivlaivfg nvlvitaiak |
| 61 | ferlqtvtny fitslacadl vmglavvpfg aahilmkmwt |
| | fgnfwcefwt sidvlcvtas |
| 121 | ietlcviavd ryfaitspfk yqslltknka rviilmvwiv |
| | sgltsflpiq mhwyrathqe |
| 181 | aincyanetc cdfftnqaya iassivsfyv plvimvfvys |
| | rvfqeakrql qkidksegrf |
| 241 | hvqnlsqveq dgrtghglrr sskfclkehk alktlgiimg |
| | tftlcwlpff ivnivhviqd |
| 301 | nlirkevyil lnwigyvnsg fnpliycrsp dfriafqell |
| | clrrsslkay gngyssngnt |
| 361 | geqsgyhveq ekenkllced lpgtedfvgh qgtvpsdnid |
| | sqgrncstnd sll |

The gene encoding either the full-length Beta-2 adrenergic receptor or the cytoplasmic loops II or loop III or helix VII is cloned, expressed and purified from E. coli or baculovirus infected cells (Hampe, et al., J Biotechnol 77:219-234(2000)) according to published procedures, incorporated into detergent micelles to simulate the cellular milieu (Min, et al., J Biol Chem 268:9400-9404(1993)). A library of up to 10¹⁷ variants of in vitro synthesized ribozymes is allowed to react with purified Beta-2 adrenergic receptor at a final concentration of 1 uM. Selection of allosterically activated ribozymes is carried out by procedures outlined in prior examples. Complexes are formed 1:1 with butoxamine or isoproterenol.

### EXAMPLE 8

### Selection of Riboreporters which are Activated by the butoxamine-Beta-2 Adrenergic Complex

1:1 complexes of butoxamine and purified Beta-2 adrenergic receptor are formed and selection of allosterically activated ribozymes is carried out by procedures outlined in prior examples.

### EXAMPLE 9

### Selection of Riboreporters which are Activated by the isoproterenol-Beta-2 Adrenergic Complex

1:1 complexes of isoproterenol and purified Beta-2 adrenergic receptor are formed and selection of allosterically activated ribozymes is carried out by procedures outlined in prior examples.

### EXAMPLE 10

### Selection for a Library of Riboreporters which Signal the Presence of all known GPCRs

The full-length gene sequences for over 400 GPCRs is known. The entire gene, or peptides derived from these sequences, is N-terminally tagged or N-/C-terminally His-tagged and cloned, expressed, and purified as described above, or synthesized by chemical means. All 400 GPCRs are produced in either a serial or parallel fashion and the purified proteins or peptides stored in buffer containing 50 mM Tris-HCl, 1 mM EDTA, 1 mM DTT, and 50-250 mM NaCl/SCN salt, pH 7 to pH 8.5, 10% glycerol or other stabilizing agent. Protein sequence and MW is verified by standard mass spectrometry.

1. Selection of Riboreporters which are Activated by GPCRs not Bound to Ligand:

A library of up to 10¹⁷ variants of in vitro synthesized ribozymes is allowed to react with purified GPCRs at a final concentration of 1 uM GPCR. Selection of allosterically activated ribozymes is carried out by procedures outlined in prior examples. Selections are carried out in parallel fashion or also can be carried out in mixed pools of anywhere from 5-10 GPCRs. In the final rounds of riboreporter selection, the RNA pools may separated into aliquots which may then be used to carry out in vitro selection against single GPCR proteins to yield unique riboreporters selective for all 400 GPCRs.

2. Selection of Riboreporters which are Activated by Ligand Bound Forms

Of the 4000 known NHRs, there are approximately 120 known ligands-GPCR pairs, described in the attached table. Stable complexes of each GPCR LBD are formed with from 1-10 equivalents of ligand. Selection of allosterically activated ribozymes is carried out by procedures outlined in prior examples. Selections are carried out in parallel fashion or also can be carried out in mixed pools of anywhere from 5-10 GPCR-ligand complexes. In the final rounds of riboreporter selection, the RNA pools may separated into aliquots which may then be used to carry out in vitro selection against single GPCR-ligand complex to yield unique riboreporters selective for all GPCR-ligand complexes.

### EXAMPLE 11

### Selection of Riboreporters Using Peptide Fragments of GPCRs.

The example describes a method to develop riboreporters that specifically recognize conformational isoforms of GPCRs that are revealed upon ligand binding.

Molecular cloning studies have identified over 400 human GPCRs, and have identified the ligands for 120. GPCRs consist of three domains: an extracellular N-terminus, a central domain of seven trans-membrane helices, and a cytoplasmic C-terminus. Activation of GPCRs is induced by ligand binding, which causes a conformational change in the receptor and exposes cytoplasmic helicies II and III, as well as helix VII. This method provides for generation of unique biosensors for each GPCR. Representative GPCR peptide and polypeptides are presented in Examples 12 and 13.

The biosensors described in this invention include riboreporters such as allosteric ribozymes (AR), including hammerhead, hairpin, L1 ligase or group 1 intron ribozymes and the like, or the riboreporter may derived from aptamer beacons or signaling aptamers, and of which transducer molecular recognition into a detectible signal. In one embodiment, Riboreporters specific for GPCRs are generated by in vitro selection for recognition of peptide fragments of the GPCRs comprising regions of the cytoplasmic helices II and III, as well as helix VII. Exemplary suitable GPCR peptide fragments are presented in Example 11. Riboreporters which recognize cytoplasmic helices II and III, as well as helix VII are then capable of recognizing cytoplasmic helices II and III, as well as helix VII within the context of the full length protein and hence recognize the activated state of the GPCR. Examples of the used of peptide fragments to generate riboreporters which recognize the full length protein are known in the art and are incorporated herein. See for example data on Riboreporter selection and recognition of HIV rev peptide and full length protein [Michael Robertson, 2001, University of Texas, Austin, Ph.D. Dissertation]. In the case of HIV rev, unique peptide sequences are recognized both as free peptides and in the context of the full protein.

### EXAMPLE 12

### GPCR Peptide Fragments

| | |
|---|---|
| >G(S)-1 | |
| mgclgnskte dqrneekaqr eankkiekql qkdkqvyrat hrllllgage sgkstivkqm | (SEQ ID NO:13) |
| rilhvngfng eggeedpqaa rsnsdgekat kvqdiknnlk eaietivaam snlvppvela | |
| npenqfrvdy ilsvmnvpdf dfppefyeha kalwedegvr acyersneyq lidcaqyfld | |
| kidvikqady vpsdqdllrc rvltsgifet kfqvdkvnfh mfdvggqrde rrkwiqcfnd | |
| vtaiifvvas ssynmvired nqtnrlqeal nlfksiwnnr wlrtisvilf lnkqdllaek | |
| vlagkskied yfpefarytt pedatpepge dprvtrakyf irdefirist asgdgrhycy | |
| phftcavdte nirrvfndcr diiqrmhlrq yell | |
| >G(S)-2 | |
| mgclgnskte dqrneekaqr eankkiekql qkdkqvyrat hrllllgage sgkstivkqm | (SEQ ID NO:14) |
| rilhvngfng eggeedpqaa rsnsdgseka tkvqdiknnl keaietivaa msnlvppvel | |
| anpenqfrvd yilsvmnvpd fdfppefyeh akalwedegv racyersney qlidcaqyfl | |
| dkidvikqad yvpsdqdllr crvltsgife tkfqvdkvnf hmfdvggqrd errkwiqcfn | |
| dvtaiifvva sssynmvire dnqtnrlqea lnlfksiwnn rwlrtisvil flnkqdllae | |
| kvlagkskie dyfpefaryt tpedatpepg edprvtraky firdeflris tasgdgrhyc | |
| yphftcavdt enirrvfndc rdiiqrmhlr qyell | |
| >G(S)-3 | |
| mgclgnskte dqrneekaqr eankkiekql qkdkqvyrat hrllllgage sgkstivkqm | (SEQ ID NO:15) |
| rilhvngfng dekatkvqdi knnlkeaiet ivaamsnlvp pvelanpenq frvdyilsvm | |
| nvpdfdfppe fyehakalwe degvracyer sneyqlidca qyfldkidvi kqadyvpsdq | |
| dllrcrvlts gifetkfqvd kvnfhmfdvg gqrderrkwi qcfndvtaii fvvasssynm | |
| virednqtnr lqealnlfks iwnnrwlrti svilflnkqd llaekvlagk skiedyfpef | |
| aryttpedat pepgedprvt rakyfirdef lristasgdg rhycyphftc avdtenirrv | |
| fndcrdiiqr rnhlrqyell | |
| >G(S)-4 | |
| mgcignskte dqrneekaqr eankkiekql qkdkqvyrat hrllllgage sgkstivkqm | (SEQ ID NO:16) |
| rilhvngfng dsekatkvqd iknnlkeaie tivaamsnlv ppvelanpen qfrvdyilsv | |
| mnvpdfdfpp efyehakalw edegvracye rsneyqlidc aqyfldkidv ikqadyvpsd | |
| qdllrcrvlt sgifetkfqv dkvnfhmfdv ggqrderrkw iqcfndvtai ifvvasssyn | |
| mvirednqtn rlqealnlfk siwnnrwlrt isvilflnkq dllaekvlag kskiedyfpe | |
| faryttpeda tpepgedprv trakyfirde flristasgd grhycyphft cavdtenirr | |
| vfndcrdiiq rmhlrqyell | |
| >G(s)-x1 | |
| meisgppfei gsapagvddt pvnmdsppia ldgppikvsg apdkreraer ppveeeaaem | (SEQ ID NO:17) |
| egaadaaegg kvpspgygsp aagaasadta araapaapad pdsgatpedp dsgtapadpd | |
| sgafaadpds gaapaapadp dsgaapdapa dpdsgaapda padpdagaap eapaapaaae | |
| traahvapaa pdagaptapa asatraaqvr raasaapasg arrkihlrpp speiqaadpp | |
| tprptrasaw rgksessrgr rvyydegvas sdddssgdes ddgtsgclrw fqhrrnrrrr | |
| kpqrnllrnf lvqafggcfg rsespqpkas rslkvkkvpl aekrrqmrke alekraqkra | |
| ekkrsklidk qlqdekmgym cthrllll | |
| >g-olf | |
| mgclggnskt tedqgvdeke rreankkiek qlqkerlayk athrllllga gesgkstivk | (SEQ ID NO:18) |
| qmrilhvngf npeekkqkil dirknvkdai vtivsamsti ippvplanpe nqfrsdyiks | |
| iapitdfeys qeffdhvkkl wddegvkacf ersneyqlid caqyflerid svslvdytpt | |
| dqdllrcrvl tsgifetrfq vdkvnfhmfd vggqrderrk wiqcfndvta iiyvaacssy | |
| nmvirednnt nrlresldlf esiwnnrwlr tisiilflnk qdmlaekvla gkskiedyfp | |
| eyanytvped atpdagedpk vtrakffird lflristatg dgkhycyphf tcavdtenir | |
| rvfndcrdii qrmhlkqyel l | |
| >I1 | |
| mgctlsaedk aaverskmid rnlredgeka arevkllllg agesgkstiv kqmkiiheag | (SEQ ID NO:19) |
| yseeeckqyk avvysntiqs iiaiiramgr lkidfgdsar addarqlfvl agaaeegfmt | |
| aelagvikrl wkdsgvqacf nrsreyqlnd saayylndld riaqpnyipt qqdvlrtrvk | |
| ttgivethft fkdlhfkmfd vggqrserkk wihcfegvta iifcvalsdy dlvlaedeem | |
| nrmhesmklf dsicnnkwft dtsiilflnk kdlfeekikk spiticypey agsntyeeaa | |
| ayiqcqfedl nkrkdtkeiy thftcatdtk nvqfvfdavt dviiknnlkd cglf | |
| >I2 | |
| mgctvsaedk aaaerskmid knlredgeka arevkllllg agesgkstiv kqmkiihedg | (SEQ ID NO:20) |
| yseeecrqyr avvysntiqs imaivkamgn lqidfadpsr addarqlfal sctaeeqgvl | |
| pddlsgvirr lwadhgvqac fgrsreyqln dsaayylndl eriaqsdyip tqqdvlrtrv | |
| kttgivethf tfkdlhfkmf dvggqrserk kwihcfegvt aiifcvalsa ydlvlaedee | |
| mnrmhesmkl fdsicnnkwf tdtsiilfln kkdlfeekit hsplticfpe ytgankydea | |
| asyiqskfed lnkrkdtkei ythftcatdt knvqfvfdav tdviiknnlk dcglf | |
| >I3 | |
| mgctlsaedk aaverskmid rnlredgeka akevkllllg agesgkstiv kqmkiihedg | (SEQ ID NO:21) |
| ysedeckqyk vvvysntiqs iiaiiramgr lkidfgeaar addarqlfvl agsaeegvmt | |
| pelagvikrl wrdggvqacf srsreyqlnd sasyylndld risqsnyipt qqdvlrtrvk | |
| ttgivethft fkdlyfkmfd vggqrserkk wihcfegvta iifcvalsdy dlvlaedeem | |
| nrmhesmklf dsicnnkwft etsiilflnk kdlfeekikr spiticypey tgsntyeeaa | |
| ayiqcqfedl nrrkdtkeiy thftcatdtk nvqfvfdavt dviiknnlke cgly | |
| >G01 | |
| mgctlsaeer aalerskaie knikedgisa akdvkllllg agesgkstiv kqmkiihedg | (SEQ ID NO:22) |
| fsgedvkqyk pvvysntiqs laaivramdt lgieygdker kadakmvcdv vsrmedtepf | |
| saellsammr lwgdsgiqec fnrsreyqln dsakyyldsl drigaadyqp teqdilrtrv | |
| kttgivethf tfknlhfrlf dvggqrserk kwihcfedvt aiifcvalsg ydqvlhedet | |
| tnrmheslml fdsicnnkff idtsiilfln kkdltgekik ksplticfpe ytgpntyeda | |
| aayiqaqfes knrspnkeiy chmtcatdtn niqvvfdavt diiiannlrg cgly | |
| >g02 | |
| mgctvsaedk aaaerskmid knlredgeka arevkllllg agesgkstiv kqmkiihedg | (SEQ ID NO:23) |
| yseeecrqyr avvysntiqs imaivkamgn lqidfadpsr addarqlfal sctaeeqgvl | |
| pddlsgvirr lwadhgvqac fgrsreyqln dsaayylndl eriaqsdyip tqqdvlrtrv | |
| kttgivethf tfkdlhfkmf dvggqrserk kwihcfegvt aiifcvalsa ydlvlaedee | |
| mnrmhesmkl fdsicnnkwf tdtsiilfln kkdlfeekit hsplticfpe ytgankydea | |
| asyiqskfed lnkrkdtkei ythftcatdt knvqfvfdav tdviikrmlk dcglf | |
| >G(T-1) | |
| mgagasaeek hsrelekklk edaekdartv kllllgages gkstivkqmk iihqdgysle | (SEQ ID NO:24) |
| eclefiaiiy gntlqsilai vramttlniq ygdsarqdda rklmhmadti eegtmpkems | |
| diiqrlwkds giqacferas eyqlndsagy ylsdlerlvt pgyvpteqdv lrsrvkttgi | |
| ietqfsfkdl nfrmfdvggq rserkkwihc fegvtciifi aalsaydmvl veddevnrmh | |
| eslhlfnsic nhryfattsi vlflnkkdvf fekikkahls icfpdydgpn tyedagnyik | |
| vqflelnmrr dvkeiyshmt catdtqnvkf vfdavtdiii kenlkdcglf | |
| >G(T-2) | |
| mgsgasaedk elakrskele kklqedadke aktvkllllg agesgkstiv kqmkiihqdg | (SEQ ID NO:25) |
| yspeeclefk aiiygnvlqs ilaiiramtt lgidyaepsc addgrqlnnl adsieegtmp | |
| pelvevirrl wkdggvqacf eraaeyqlnd sasyylnqle ritdpeylps eqdvlrsrvk | |
| ttgiietkfs vkdlnfrmfd vggqrserkk wihcfegvtc iifcaalsay dmvlveddev | |
| nrmheslhlf nsicnhkffa atsivlflnk kdlfeekikk vhlsicfpey dgnnsyddag | |
| nyiksqfldl nmrkdvkeiy shmtcatdtq nvkfvfdavt diiikenlkd cglf | |
| >G(Z) | |
| mvflsgnasd ssnctqppap vniskaillg vilgglilfg vlgnilvils vachrhlhsv | (SEQ ID NO:26) |
| thyyivnlav adllltstvl pfsaifevlg ywafgrvfon iwaavdvlcc tasimglcii | |
| sidryigvsh plryptivtq rrglmallcv walslvisig plfgwrgpap edeticqine | |
| epgyvlfsal gsfylplaii lvmycrvyvv akresrglks glktdksdse qvtlrihrkn | |
| apaggsgmas aktkthfsvr llkfsrekka aktlgivvgc fvlcwlpffl vmpigsffpd | |
| fkpsetvfki vfwlgylnsc inpiiyposs qefkkafqnv lriqclcrkq sskhalgytl | |
| hppsqavegq hkdmvripvg sreafygisr tdgvcewkff ssmprgsari tvskdqssct | |
| tarvrsksfl qvcccvepst psldknhqvp tikvhtisls engeev | |
| >G(Q) | |
| macclseeak earrindeie rqlrrdkrda rrelkllllg tgesgkstfi kqmriihgsg | (SEQ ID NO:27) |
| ysdedkrgft klvygnifta mqamiramdt lkipykyehn kahaqlvrev dvekvsafen | |
| pyvdaikslw ndpgiqecyd rrreyqlsds tkyylndldr vadpaylptq qdvlrvrvpt | |
| tgiieypfdl qsvifrmvdv ggqrserrkw ihcfenvtsi mflvalseyd qvlvesdnen | |
| rmeeskalfr tiitypwfqn ssvilflnkk dlleekimys hlvdyfpeyd gpqrdaqaar | |
| efilkmfvdl npdsdkiiys hftcatdten irfvfaavkd tilqlnlkey nay | |
| >G(Y-11) | |
| mtlesmmacc lsdevkeskr inaeiekqlr rdkrdarrel kllllgtges gkstfikqmr | (SEQ ID NO:28) |
| iihgagysee dkrgftklvy qniftamqam irametlkil ykyeqnkana llirevdvek | |
| vttfehqyvs aiktlwedpg iqecydrrre yqlsdsakyy ltdvdriatl gylptqqdvl | |
| rvrvpttgii eypfdlenii frmvdvggqr serrkwihcf envtsimflv alseydqvlv | |
| esdnenrmee skalfrtiit ypwfqnssvi lflnkkdlle dkilyshlvd yfpefdgpqr | |
| daqaarefil kmfvdlnpds dkiiyshftc atdtenirfv faavkdtilq lnlkeynlv | |
| >G(Y-12) | |
| msgvvrtlsr cllpaeagga rerragsgar daerearrrs rdidallare rravrrlvki | (SEQ ID NO:29) |
| lllgagesgk stflkqmrii hgrefdqkal lefrdtifdn ilkgsrvlvd ardklgipwq | |
| ysenekhgmf imafenkagl pvepatfqly vpalsalwrd sgireafsrr sefqlgesvk | |
| yfldnldrig qlnyfpskqd illarkatkg ivehdfvikk ipfkmvdvgg qrsqrqkwfq | |
| cfdgitsilf mvssseydqv lmedrrtnrl vesmnifeti vnnklffnvs iilflnkmdl | |
| lvekvktvsi kkhfpdfrgd phqledvqry lvqcfdrkrr nrskplfhhf ttaidtenvr | |
| fvfhavkdti lqenlkdiml q | |
| >G(Y-13 | |
| madflpsrsv lsvcfpgcll tsgeaeqqrk skeidkclsr ektyvkrlvk illlgagesg | (SEQ ID NO:30) |
| kstflkqmri ihgqdfdqra reefrptiys nvikgmrvlv dareklhipw gdnsnqqhgd | |
| kmmsfdtrap maaqgmvetr vflqylpair alwadsgiqn aydrrrefql gesvkyfldn | |
| ldklgepdyi psqqdillar rptkgiheyd feiknvpfkm ldvggqrser krwfecfdsv | |
| tsilflvsss efdqvlmedr ltnrltesln ifetivnnrv fsnvsiilfl nktdlleekv | |
| qivsikdyfl efegdphclr dvqkflvecf rnkrrdqqqk plyhhfttai ntenirlvfr | |
| dvkdtilhdn lkqlmlq | |
| >G(Y-14) | |
| magccclsae ekesqrisae ierqlrrdkk darrelklll lgtgesgkst fikqmriihg | (SEQ ID NO:31) |
| sgysdedrkg ftklvyqnif tamqamiram dtlriqyvce qnkenaqiir evevdkvsml | |
| sreqveaikq lwqdpgiqec ydrrreyqls dsakyyltdi driatpsfvp tqqdvlrvrv | |
| pttgiieypf dleniifrmv dvggqrserr kwihcfesvt siiflvalse ydqvlaecdn | |
| enrmeeskal fktiitypwf lnssvilfln kkdlleekim yshlisyfpe ytgpkqdvra | |
| ardfilklyq dqnpdkekvi yshftcatdt dnirfvfaav kdtilqlnlr efnlv | |
| >G(Y-15) | |
| marsltwrcc pwcltedeka aarvdqeinr illeqkkqdr gelkllllgp gesgkstfik | (SEQ ID NO:32) |
| qmriihgagy seeerkgfrp lvyqnifvsm ramieamerl qipfsrpesk hhaslvmsqd | |
| pykvttfekr yaaamqwlwr dagiracyer rrefhlldsa vyylshleri teegyvptaq | |
| dvlrsrmptt gineycfsvq ktnlrivdvg gqkserkkwi hcfenviali ylaslseydq | |
| cleennqenr mkeslalfgt ilelpwfkst svilflnktd ileekiptsh latyfpsfqg | |
| pkqdaeaakr fildmytrmy tgcvdgpegs kkgarsrrlf shytcatdtq nirkvfkdvr | |
| dsvlarylde inll | |
| >Gusducin | |
| mfdvggqrse rkkwihcfeg vtciifcaal saydmvlved eevnrmhesl hlfnsicnhk | (SEQ ID NO:33) |
| yfsttsivlf lnkkdifqek vtkvhlsicf peytgpntfe dagnyiknqf ldlnlkkedk | |
| eiyshmtcat dtqnvkfvfd avtdiiiken lkdcglf | |

### EXAMPLE 13

### GPCR Polypeptides

| | |
|---|---|
| ref\|NP_006134.1\| | G protein-coupled receptor 19 [Homo sapiens] |
| ref\|XP_049562.1\| | G protein-coupled receptor 19 [Homo sapiens] |
| ref\|NP_004876.1\| | neuropeptide G protein-coupled receptor; n . . . |
| ref\|XP_011102.1\| | 46228 [Homo sapiens] >gi\|14723215 \|ref\|XP_0 . . . |
| ref\|NP_057624.1\| | G protein-coupled receptor 72; reserved; G . . . |
| ref\|XP_011520.3\| | orexin receptor 2 [Homo sapiens] |
| ref\|NP_001517.1\| | orexin receptor 2 [Homo sapiens] |
| ref\|NP_001471.1\| | galanin receptor 1; Galanin receptor [Homo . . . |
| ref\|NP_071429.1\| | neuropeptide FF 1; RFamide-related peptide . . . |
| ref\|XP_005747.4\| | tachykinin receptor 2 [Homo sapiens] |
| ref\|NP_001048.1\| | tachykinin receptor 2; Tachykinin receptor . . . |
| ref\|NP_000901.1\| | neuropeptide Y receptor Y2 [Homo sapiens] . . . |
| ref\|NP_003848.1\| | galanin receptor 2 [Homo sapiens] >gi\|1365 . . . |
| ref\|XP_004030.2\| | adrenergic, beta-2-, receptor, surface [Ho . . . |
| ref\|NP_000015.1\| | adrenergic, beta-2-, receptor, surface [Ho . . . |
| ref\|XP_001777.1\| | orexin receptor 1 [Homo sapiens] |
| ref\|XP_011871.3\| | neuropeptide FF 1; RFamide-related peptide . . . |
| ref\|NP_001516.1\| | orexin receptor 1 [Homo sapiens] |
| ref\|NP_001041.1\| | somatostatin receptor 2 [Homo sapiens] >gi . . . |
| ref\|NP_001040.1\| | somatostatin receptor 1 [Homo sapiens] >gi . . . |
| ref\|NP_001044.1\| | somatostatin receptor 5 [Homo sapiens] |
| ref\|XP_012565.1\| | somatostatin receptor 5 [Homo sapiens] |
| ref\|NP_115940.1\| | G protein-coupled receptor; G protein-coup . . . |
| ref\|XP_037563.1\| | G protein-coupled receptor [Homo sapiens] |
| ref\|NP_001050.1\| | tachykinin receptor 3; NK-3 receptor; neur . . . |
| ref\|XP_011942.1\| | prolactin-releasing hormone receptor [Homo . . . |
| ref\|XP_017624.1\| | G protein-coupled receptor 58 [Homo sapiens] |
| ref\|NP_004239.1\| | prolactin-releasing hormone receptor [Homo . . . |
| ref\|NP_071640.1\| | histamine receptor H2; gastric receptor 1 . . . |
| ref\|NP_055441.1\| | G protein-coupled receptor 58 [Homo sapiens] |
| ref\|XP_009594.2\| | somatostatin receptor 4 [Homo sapiens] |
| ref\|NP_003605.1\| | galanin receptor 3; galanin receptor, fami . . . |
| ref\|NP_001049.1\| | tachykinin receptor 1, isoform long; Tachy . . . |
| ref\|XP_039747.1\| | opioid receptor, mu 1 [Homo sapiens] >gi\|1 . . . |
| ref\|NP_000905.1\| | opioid receptor, mu 1 [Homo sapiens] |
| ref\|XP_004341.2\| | 53355 [Homo sapiens] |
| ref\|XP_052174.1\| | 50635 [Homo sapiens] |
| ref\|XP_052175.1\| | 5-hydroxytryptamine (serotonin) receptor 4 . . . |
| ref\|XP_052165.1\| | 5-hydroxytryptamine (serotonin) receptor 4 . . . |
| ref\|XP_052164.1\| | 50636 [Homo sapiens] >gi\|14732317\|ref\|XP_0 . . . |
| ref\|NP_000861.1\| | 5-hydroxytryptamine (serotonin) receptor 4 . . . |
| ref\|NP_001043.1\| | somatostatin receptor 4 [Homo sapiens] |
| ref\|NP_000721.1\| | cholecystokinin A receptor [Homo sapiens] . . . |
| ref\|NP_006670.1\| | putative opioid receptor, neuromedin K (ne . . . |
| ref\|NP_055442.1\| | G protein-coupled receptor 57 [Homo sapiens] |
| ref\|NP_000698.1\| | arginine vasopressin receptor 1B; arginine . . . |
| ref\|NP_001718.1\| | bombesin-like receptor 3 [Homo sapiens] >g . . . |
| ref\|XP_040306.1\| | similar to |
| | SOMATOSTATIN RECEPTOR TYPE 2 (S . . . |
| ref\|NP_056542.1\| | tachykinin receptor 1, isoform short; Tach . . . |
| ref\|NP_001042.1\| | somatostatin receptor 3 [Homo sapiens] >gi . . . |
| ref\|NP_000722.1\| | cholecystokinin B receptor [Homo sapiens] |
| ref\|NP_000789.1\| | dopamine receptor D5; Dopamine receptor D1 . . . |
| ref\|NP_000612.1\| | 5-hydroxytryptamine (serotonin) receptor 2 . . . |
| ref\|NP_004215.1\| | G protein-coupled receptor 50 [Homo sapiens] |
| ref\|XP_010228.2\| | G protein-coupled receptor 50 [Homo sapiens] |
| ref\|NP_000907.1\| | oxytocin receptor [Homo sapiens] |
| ref\|XP_052179.1\| | oxytocin receptor [Homo sapiens] >gi\|14725 . . . |
| ref\|NP_005192.1\| | chemokine (C-C motif) receptor 8; chemokin . . . |
| ref\|NP_000670.1\| | adrenergic, alpha-1B-, receptor; adrenergi . . . |
| ref\|XP_046588.1\| | G protein-coupled receptor slt [Homo sapie . . . |
| ref\|NP_000671.1\| | adrenergic, alpha-1A-, receptor; adrenergi . . . |
| ref\|NP_110411.1\| | brain expressed G-protein-coupled receptor . . . |
| ref\|XP_003199.2\| | growth hormone secretagogue receptor [Homo . . . |
| ref\|XP_017623.1\| | G protein-coupled receptor 57 [Homo sapiens] |
| ref\|NP_005949.1\| | melatonin receptor 1A; melatonin receptor . . . |
| ref\|NP_115892.1\| | G protein-coupled receptor slt; melanin-co . . . |
| ref\|NP_000903.1\| | opioid receptor, kappa 1; Opiate receptor, . . . |
| ref\|NP_000900.1\| | neuropeptide Y receptor Y1; Neuropeptide Y . . . |
| ref\|XP_011716.2\| | similar to opioid receptor, kappa 1; Opiat . . . |
| ref\|XP_011707.2\| | adrenergic, alpha-1A-, receptor [Homo sapi . . . |
| ref\|XP_048085.1\| | adrenergic, alpha-1A-, receptor [Homo sapi . . . |
| ref\|XP_048084.1\| | adrenergic, alpha-1A-, receptor [Homo sapi . . . |
| ref\|NP_000785.1\| | dopamine receptor D1 [Homo sapiens] |
| ref\|XP_048082.1\| | adrenergic, alpha-1A-, receptor [Homo sapi . . . |
| ref\|NP_003292.1\| | thyrotropin-releasing hormone receptor [Ho . . . |
| ref\|NP_005305.1\| | gastrin-releasing peptide receptor [Homo s . . . |
| ref\|XP_006335.4\| | dopamine receptor D2 [Homo sapiens] |
| ref\|XP_006334.3\| | dopamine receptor D2longer [Homo sapiens] |
| ref\|NP_000786.1\| | dopamine receptor D2 [Homo sapiens] >gi\|14 . . . |
| ref\|XP_036647.1\| | dopamine receptor D2 [Homo sapiens] >gi\|14 . . . |
| ref\|XP_041422.1\| | similar to dopamine receptor D2 (H. sapien . . . |
| ref\|NP_057658.1\| | dopamine receptor D2longer [Homo sapiens] . . . |
| ref\|NP_000697.1\| | arginine vasopressin receptor 1A; Vla vaso . . . |
| ref\|NP_002502.1\| | neuromedin B receptor [Homo sapiens] |
| ref\|XP_018475.1\| | neuromedin B receptor [Homo sapiens] |
| ref\|NP_062874.1\| | 5-hydroxytryptamine receptor 7, isoform b; . . . |
| ref\|NP_000730.1\| | cholinergic receptor, muscarinic 2; muscar . . . |
| ref\|NP_062873.1\| | 5-hydroxytryptamine receptor 7, isoform d; . . . |
| ref\|NP_000863.1\| | 5-hydroxytryptamine receptor 7, isoform a; . . . |
| ref\|NP_000667.1\| | adenosine A2b receptor [Homo sapiens] >gi\| . . . |
| ref\|NP_000675.1\| | beta-1-adrenergic receptor [Homo sapiens] . . . |
| ref\|NP_005963.1\| | pancreatic polypeptide receptor 1 [Homo sa . . . |
| ref\|NP_000732.1\| | cholinergic receptor, muscarinic 4; muscar . . . |
| ref\|XP_039923.1\| | 44527 [Homo sapiens] |
| ref\|NP_000787.1\| | dopamine receptor D3 [Homo sapiens] |
| ref\|XP_011027.3\| | dopamine receptor D3 [Homo sapiens] |
| ref\|NP_061822.1\| | G protein-coupled receptor 14 [Homo sapiens] |
| ref\|NP_000731.1\| | cholinergic receptor, muscarinic 3; muscar . . . |
| ref\|NP_000669.1\| | adrenergic, alpha-1D-, receptor; adrenergi . . . |
| ref\|NP_005282.1\| | G protein-coupled receptor 17 [Homo sapien . . . |
| ref\|XP_048332.1\| | similar to purinergic receptor (family A g . . . |
| ref\|XP_041897.1\| | similar to G protein-coupled receptor 17 ( . . . |
| ref\|NP_005152.1\| | angiotensin receptor-like 1 [Homo sapiens] . . . |
| ref\|NP_000016.1\| | adrenergic, beta-3-, receptor [Homo sapien . . . |
| ref\|NP_001286.1\| | chemokine (C-C motif) receptor 1; macropha . . . |
| ref\|NP_000902.1\| | opioid receptor, delta 1 [Homo sapiens] |
| ref\|NP_005758.1\| | purinergic receptor (family A group 5) [Ho . . . |
| ref\|XP_006296.1\| | cholinergic receptor, muscarinic 4 [Homo s . . . |
| ref\|XP_006058.1\| | similar to |
| | MUSCARINIC ACETYLCHOLINE RECEPT . . . |
| ref\|NP_000729.1\| | cholinergic receptor, muscarinic 1; muscar . . . |
| ref\|NP_000904.1\| | opiate receptor-like 1; opiod receptor-lik . . . |
| ref\|NP_006630.1\| | cysteinyl leukotriene receptor 1 [Homo sap . . . |
| ref\|NP_001828.1\| | chemokine (C-C motif) receptor 3 [Homo sap . . . |
| ref\|NP_000530.1\| | rhodopsin; rhodopsin (retinitis pigmentosa . . . |
| ref\|NP_009154.1\| | putative G protein coupled receptor [Homo . . . |
| ref\|XP_009882.2\| | adenosine A2a receptor [Homo sapiens] |
| ref\|XP_045486.1\| | adrenergic, alpha-1D-, receptor [Homo sapi . . . |
| ref\|NP_000397.1\| | gonadotropin-releasing hormone receptor; g . . . |
| ref\|NP_000666.2\| | adenosine A2a receptor; adenosine A2 recep . . . |
| ref\|XP_002299.1\| | G protein-coupled receptor 45 [Homo sapiens] |
| ref\|NP_003958.1\| | putative neurotransmitter receptor [Homo s . . . |
| ref\|NP_000854.1\| | 5-hydroxytryptamine (serotonin) receptor 1 . . . |
| ref\|XP_001811.2\| | opioid receptor, delta 1 [Homo sapiens] >g . . . |
| ref\|NP_005950.1\| | melatonin receptor 1B; melatonin receptor . . . |
| ref\|NP_001548.1\| | interleukin 8 receptor, beta [Homo sapiens] |
| ref\|NP_064552.1\| | neuromedin U receptor 2 [Homo sapiens] |
| ref\|NP_009158.1\| | G protein-coupled receptor 45 [Homo sapiens] |
| ref\|NP_000625.1\| | interleukin 8 receptor, alpha; chemokine ( . . . |
| ref\|XP_017622.1\| | putative neurotransmitter receptor [Homo s . . . |
| ref\|NP_036257.1\| | cholinergic receptor, muscarinic 5; muscar . . . |
| ref\|NP_000743.1\| | leukotriene b4 receptor (chemokine recepto . . . |
| ref\|NP_000788.1\| | dopamine receptor D4 [Homo sapiens] |
| ref\|XP_006145.2\| | dopamine receptor D4 [Homo sapiens] |
| ref\|XP_001543.1\| | G protein-coupled receptor 52 [Homo sapiens] |
| ref\|XP_009663.1\| | G protein-coupled receptor 8 [Homo sapiens] |
| ref\|XP_007212.1\| | purinergic receptor (family A group 5) [Ho . . . |
| ref\|NP_006165.1\| | neuropeptide Y receptor Y5 [Homo sapiens] |
| ref\|NP_002522.1\| | neurotensin receptor 1 [Homo sapiens] |
| ref\|XP_003692.2\| | 5-hydroxytryptamine (serotonin) receptor 1 . . . |
| ref\|XP_009612.2\| | neurotensin receptor 1 [Homo sapiens] |
| ref\|NP_005277.1\| | G protein-coupled receptor 8 [Homo sapiens] |
| ref\|NP_000515.1\| | 5-hydroxytryptamine (serotonin) receptor 1 . . . |
| ref\|NP_001707.1\| | Burkitt lymphoma receptor 1, isoform 1; C- . . . |
| ref\|NP_005675.1\| | G protein-coupled receptor 52 [Homo sapiens] |
| ref\|NP_000852.1\| | histamine receptor H1; histamine receptor, . . . |
| ref\|NP_000639.1\| | chemokine (C-C motif) receptor 2; chemokin . . . |
| ref\|NP_116743.1\| | Burkitt lymphoma receptor 1, isoform 2; C- . . . |
| ref\|NP_006574.1\| | retinal pigment epithelium-derived rhodops . . . |
| ref\|NP_031395.1\| | G-protein coupled receptor [Homo sapiens] . . . |
| ref\|XP_009373.1\| | formyl peptide receptor-like 2 [Homo sapie . . . |
| ref\|NP_076917.1\| | 5-hydroxytryptamine (serotonin) receptor 5 . . . |
| ref\|XP_005280.2\| | G protein-coupled receptor 7 [Homo sapiens] |
| ref\|XP_039818.1\| | G protein-coupled receptor 91 [Homo sapien . . . |
| ref\|NP_006164.1\| | neuropeptide Y receptor Y6 (pseudogene) [H . . . |
| ref\|XP_012748.2\| | galanin receptor 1 [Homo sapiens] >gi\|1476 . . . |
| ref\|NP_000862.1\| | 5-hydroxytryptamine (serotonin) receptor 6 . . . |
| ref\|NP_005281.1\| | G protein-coupled receptor 15 [Homo sapien . . . |
| ref\|XP_010406.1\| | angiotensin receptor 2 [Homo sapiens] >gi\| . . . |
| ref\|NP_001328.1\| | chemokine (C-X3-C) receptor 1; chemokine ( . . . |
| ref\|NP_002554.1\| | purinergic receptor P2Y, G-protein coupled . . . |
| ref\|NP_004942.1\| | EBV-induced G protein-coupled receptor 2; . . . |
| ref\|NP_000672.1\| | adrenergic, alpha-2A-, receptor [Homo sapi . . . |
| ref\|XP_005827.3\| | beta-1-adrenergic receptor [Homo sapiens] |
| ref\|NP_005288.1\| | G protein-coupled receptor 24 [Homo sapien . . . |
| ref\|NP_002021.2\| | formyl peptide receptor-like 2 [Homo sapie . . . |
| ref\|NP_005276.1\| | G protein-coupled receptor 7 [Homo sapiens] |
| ref\|XP_010009.2\| | similar to somatostatin receptor-like prot . . . |
| ref\|NP_003458.1\| | chemokine (C-X-C motif), receptor 4 (fusin . . . |
| ref\|NP_000673.1\| | adrenergic, alpha-2B-, receptor [Homo sapi . . . |
| ref\|XP_051229.1\| | similar to C-X-C |
| | CHEMOKINE RECEPTOR TYPE 4 . . . |
| ref\|NP_001453.1\| | formyl peptide receptor-like 1; lipoxin A4 . . . |
| ref\|NP_006047.1\| | G protein-coupled receptor 66 [Homo sapien . . . |
| ref\|NP_000856.1\| | 5-hydroxytryptamine (serotonin) receptor 1 . . . |
| ref\|XP_048737.1\| | 41064 [Homo sapiens] |
| ref\|NP_005499.1\| | chemokine (C-C motif) receptor 4; chemokin . . . |
| ref\|NP_000638.1\| | chemokine (C-C motif) receptor 2; chemokin . . . |
| ref\|XP_009664.1\| | opiate receptor-like 1 [Homo sapiens] >gi\| . . . |
| ref\|NP_005285.1\| | G protein-coupled receptor 21 [Homo sapien . . . |
| ref\|XP_009561.2\| | 34426 [Homo sapiens] |
| ref\|NP_004063.1\| | chemokine-like receptor 1 [Homo sapiens] > . . . |
| ref\|XP_035769.1\| | chemokine-like receptor 1 [Homo sapiens] |
| ref\|NP_000855.1\| | 5-hydroxytryptamine (serotonin) receptor 1 . . . |
| ref\|NP_000045.1\| | arginine vasopressin receptor 2 [Homo sapi . . . |
| ref\|NP_064445.1\| | opsin 1 (cone pigments), long-wave-sensiti . . . |
| ref\|XP_048964.1\| | similar to |
| | PROBABLE G PROTEIN-COUPLED RECE . . . |
| ref\|NP_000665.1\| | adenosine A1 receptor [Homo sapiens] >gi\|1 . . . |
| ref\|XP_011880.1\| | similar to pancreatic polypeptide receptor . . . |
| ref\|NP_000676.1\| | angiotensin receptor 1; angiotensin recept . . . |
| ref\|NP_000674.1\| | adrenergic, alpha-2C-, receptor [Homo sapi . . . |
| ref\|XP_002705.3\| | G protein-coupled receptor 17 [Homo sapiens] |
| ref\|NP_000677.1\| | angiotensin receptor 2 [Homo sapiens] |
| ref\|XP_004279.1\| | chemokine (C-C motif) receptor 6 [Homo sap . . . |
| ref\|NP_000504.1\| | opsin 1 (cone pigments), medium-wave-sensi . . . |
| ref\|XP_033840.1\| | similar to chemokine (C-C motif) receptor . . . |
| ref\|NP_002555.1\| | purinergic receptor P2Y, G-protein coupled . . . |
| ref\|XP_006367.1\| | purinergic receptor P2Y, G-protein coupled . . . |
| ref\|NP_004358.1\| | chemokine (C-C motif) receptor 6; chemokin . . . |
| ref\|XP_045851.1\| | opsin 1 (cone pigments), short-wave-sensit . . . |
| ref\|NP_057641.1\| | orphan seven-transmembrane receptor, chemo . . . |
| ref\|XP_003251.1\| | chemokine (C-C motif) receptor 9 [Homo sap . . . |
| ref\|NP_001699.1\| | opsin 1 (cone pigments), short-wave-sensit . . . |
| ref\|XP_002838.5\| | similar to C-C |
| | CHEMOKINE RECEPTOR TYPE 11 . . . |
| ref\|NP_000570.1\| | chemokine (C-C motif) receptor 5; chemokin . . . |
| ref\|NP_006632.2\| | chemokine (C-C motif) receptor 9, isoform . . . |
| ref\|NP_000859.1\| | 5-hydroxytryptamine (serotonin) receptor 2 . . . |
| ref\|NP_115942.1\| | putative purinergic receptor [Homo sapiens . . . |
| ref\|NP_001497.1\| | G protein-coupled receptor 32 [Homo sapien . . . |
| ref\|NP_061843.1\| | G protein-coupled receptor 85; super conse . . . |
| ref\|NP_006555.1\| | G protein-coupled receptor [Homo sapiens] . . . |
| ref\|NP_065110.1\| | cysteinyl leukotriene CysLT2 receptor; cDN . . . |
| ref\|NP_004113.1\| | growth hormone secretagogue receptor [Homo . . . |
| ref\|NP_055137.1\| | opsin 3 (encephalopsin) [Homo sapiens] |
| ref\|XP_001515.3\| | opsin 3 (encephalopsin) [Homo sapiens] >gi . . . |
| ref\|NP_005274.1\| | G protein-coupled receptor 5 [Homo sapiens . . . |
| ref\|NP_061842.1\| | super conserved receptor expressed in brai . . . |
| ref\|NP_005291.1\| | G protein-coupled receptor 34 [Homo sapien . . . |
| ref\|NP_037477.1\| | G protein-coupled receptor [Homo sapiens] . . . |
| ref\|XP_003126.1\| | chemokine binding protein 2 [Homo sapiens] . . . |
| ref\|XP_007392.1\| | G protein-coupled receptor 65 [Homo sapiens] |
| ref\|NP_005284.1\| | G protein-coupled receptor 20 [Homo sapiens] |
| ref\|NP_005287.1\| | G protein-coupled receptor 23 [Homo sapien . . . |
| ref\|NP_009195.1\| | adrenomedullin receptor; G-protein-coupled . . . |
| ref\|NP_003941.1\| | coagulation factor II (thrombin) receptor- . . . |
| ref\|NP_000701.1\| | bradykinin receptor B1 [Homo sapiens] |
| ref\|NP_000857.1\| | 5-hydroxytryptamine (serotonin) receptor 1 . . . |
| ref\|NP_057652.1\| | G-protein coupled receptor SALPR; somatost . . . |
| ref\|XP_012745.1\| | histamine H4 receptor [Homo sapiens] >gi\|1 . . . |
| ref\|NP_000858.1\| | 5-hydroxytryptamine (serotonin) receptor 2 . . . |
| ref\|NP_003599.1\| | G protein-coupled receptor 65; T-cell deat . . . |
| ref\|NP_001499.1\| | G protein-coupled receptor 39 [Homo sapien . . . |
| ref\|XP_007275.2\| | bradykinin receptor B1 [Homo sapiens] |
| ref\|XP_006230.3\| | G protein-coupled receptor 72 [Homo sapien . . . |
| ref\|XP_037208.1\| | histamine receptor H3 [Homo sapiens] |
| ref\|XP_010168.2\| | arginine vasopressin receptor 2 [Homo sapi . . . |
| ref\|NP_009163.1\| | histamine receptor H3; G protein-coupled r . . . |
| ref\|XP_037209.1\| | 34432 [Homo sapiens] >gi\|14786758\|ref\|XP_0 . . . |
| ref\|NP_005273.1\| | G protein-coupled receptor 4 [Homo sapiens . . . |
| ref\|NP_000668.1\| | adenosine A3 receptor [Homo sapiens] >gi\|1 . . . |
| ref\|XP_001499.1\| | endothelial differentiation, sphingolipid |
| ref\|NP_114142.1\| | G protein-coupled receptor 61 [Homo sapiens] . . . |
| ref\|NP_002020.1\| | formyl peptide receptor 1 [Homo sapiens] > . . . |
| ref\|XP_007108.2\| | endothelin receptor type B, isoform 1 [Hom . . . |
| ref\|NP_000106.1\| | endothelin receptor type B, isoform 1 [Hom . . . |
| ref\|NP_003982.1\| | endothelin receptor type B isoform 2 [Homo . . . |
| ref\|XP_007276.2\| | bradykinin receptor B2 [Homo sapiens] >gi\| . . . |
| ref\|NP_000614.1\| | bradykinin receptor B2 [Homo sapiens] >gi\| . . . |
| ref\|XP_001907.1\| | G protein-coupled receptor 25 [Homo sapiens] |
| ref\|XP_051522.1\| | G protein-coupled receptor [Homo sapiens] |
| ref\|NP_003476.1\| | G protein-coupled receptor 68; Ovarian can . . . |
| ref\|NP_001498.1\| | G protein-coupled receptor 38 [Homo sapien . . . |
| ref\|NP_073625.1\| | platelet ADP receptor [Homo sapiens] >gi\|1 . . . |
| ref\|NP_039226.1\| | olfactory receptor, family 10, subfamily H . . . |
| ref\|XP_040869.1\| | similar to cannabinoid receptor 1 (brain) . . . |
| ref\|NP_004145.1\| | pyrimidinergic receptor P2Y, G-protein cou . . . |
| ref\|NP_057167.1\| | central cannabinoid receptor, isoform a; C . . . |
| ref\|NP_062813.1\| | seven transmembrane receptor BLTR2; leukot . . . |
| ref\|NP_149046.1\| | olfactory receptor, family 2, subfamily B, . . . |
| ref\|NP_000943.1\| | platelet-activating factor receptor [Homo . . . |
| ref\|XP_008010.2\| | similar to |
| | MELANOCYTE STIMULATING HORMONE . . . |
| ref\|NP_063950.1\| | olfactory receptor, family 2, subfamily S, . . . |
| ref\|NP_001831.1\| | central cannabinoid receptor, isoform a; C . . . |
| ref\|XP_003761.3\| | 48895 [Homo sapiens] >gi\|14724697\|ref\|XP_0 . . . |
| ref\|NP_002377.2\| | melanocortin 1 receptor (alpha melanocyte . . . |
| ref\|XP_032638.1\| | neuromedin U receptor 2 [Homo sapiens] |
| ref\|NP_005270.1\| | G protein-coupled receptor 1 [Homo sapiens] |
| ref\|NP_005293.1\| | G protein-coupled receptor 37 (endothelin . . . |
| ref\|NP_004092.1\| | coagulation factor II (thrombin) receptor- . . . |
| ref\|NP_005289.1\| | G protein-coupled receptor 25 [Homo sapiens] |
| ref\|NP_001727.1\| | complement component 5 receptor 1 (C5a lig . . . |
| ref\|XP_002667.1\| | 41743 [Homo sapiens] |
| ref\|XP_008392.1\| | CC chemokine receptor 10 [Homo sapiens] >g . . . |
| ref\|NP_001496.1\| | G protein-coupled receptor 30; chemokine r . . . |
| ref\|NP_057686.1\| | CC chemokine receptor 10 [Homo sapiens] |
| ref\|NP_001829.1\| | chemokine (C-C motif) receptor 7; Chemokin . . . |
| ref\|XP_016412.1\| | 28082 [Homo sapiens] >gi\|14721034\|ref\|XP_0 . . . |
| ref\|NP_006009.1\| | putative chemokine receptor; GTP-binding p . . . |
| ref\|NP_000136.1\| | follicle stimulating hormone receptor; ova . . . |
| ref\|NP_002541.1\| | olfactory receptor, family 3, subfamily A, . . . |
| ref\|XP_002212.2\| | follicle stimulating hormone receptor [Hom . . . |
| ref\|NP_004769.1\| | G protein-coupled receptor 44; chemoattrac . . . |
| ref\|XP_015921.1\| | similar to putative chemokine receptor; GT . . . |
| ref\|XP_015923.1\| | putative chemokine receptor; GTP-binding p . . . |
| ref\|NP_055380.1\| | olfactory receptor, family 1, subfamily A, . . . |
| ref\|NP_065133.1\| | putative G protein-coupled receptor 92 [Ho . . . |
| ref\|XP_008716.1\| | melanocortin 4 receptor [Homo sapiens] |
| ref\|NP_039225.1\| | olfactory receptor, family 11, subfamily A . . . |
| ref\|NP_002556.1\| | pyrimidinergic receptor P2Y, G-protein cou . . . |
| ref\|NP_005903.1\| | melanocortin 4 receptor [Homo sapiens] |
| ref\|NP_005297.1\| | G protein-coupled receptor 43 [Homo sapien . . . |
| ref\|XP_012667.1\| | olfactory receptor, family 1, subfamily A, . . . |
| ref\|NP_112221.1\| | olfactory receptor, family 12, subfamily D . . . |
| ref\|NP_039227.1\| | olfactory receptor, family 10, subfamily H . . . |
| ref\|NP_059976.1\| | olfactory receptor, family 7, subfamily C, . . . |
| ref\|NP_005279.1\| | G protein-coupled receptor 12 [Homo sapien . . . |
| ref\|NP_001948.1\| | endothelin receptor type A [Homo sapiens] |
| ref\|NP_005904.1\| | melanocortin 5 receptor [Homo sapiens] >gi . . . |
| ref\|NP_110401.1\| | prostate specific G-protein coupled recept . . . |
| ref\|NP_004758.1\| | endothehin type b receptor-like protein 2 . . . |
| ref\|NP_003544.1\| | olfactory receptor, family 1, subfamily E, . . . |
| ref\|NP_000520.1\| | melanocortin 2 receptor; Melanocortin-2 re . . . |
| ref\|NP_036492.1\| | olfactory receptor, family 1, subfamily F, . . . |
| ref\|NP_002557.1\| | purinergic receptor P2Y, G-protein coupled . . . |
| ref\|XP_030219.1\| | gonadotropin-releasing hormone receptor [H . . . |
| ref\|XP_009029.4\| | purinergic receptor P2Y, G-protein coupled . . . |
| ref\|NP_036284.1\| | endothelial cell differentiation gene 7; c . . . |
| ref\|NP_039228.1\| | olfactory receptor, family 10, subfamily H . . . |
| ref\|NP_036484.1\| | olfactory receptor, family 1, subfamily A, . . . |
| ref\|XP_012668.1\| | olfactory receptor, family 1, subfamily A, . . . |
| ref\|NP_001983.1\| | coagulation factor II receptor precursor; . . . |
| ref\|NP_036501.1\| | olfactory receptor, family 2, subfamily F, . . . |
| ref\|NP_055694.1\| | putative G-protein-coupled receptor; G pro . . . |
| ref\|XP_004852.1\| | olfactory receptor, family 2, subfamily F, . . . |
| ref\|NP_000949.1\| | prostaglandin E receptor 4 (subtype EP4) [ . . . |
| ref\|NP_072093.1\| | putative leukocyte platelet-activating fac . . . |
| ref\|NP_110503.1\| | olfactory receptor, family 5, subfamily V . . . |
| ref\|XP_003907.1\| | coagulation factor II receptor precursor [ . . . |
| ref\|XP_004216.1\| | similar to olfactory receptor 89 (H. sapie . . . |
| ref\|NP_112167.1\| | olfactory receptor, family 2, subfamily J, . . . |
| ref\|NP_002542.1\| | olfactory receptor, family 3, subfamily A, . . . |
| ref\|NP_036505.1\| | olfactory receptor, family 3, subfamily A, . . . |
| ref\|NP_005292.1\| | G protein-coupled receptor 35 [Homo sapiens] |
| ref\|NP_004045.1\| | complement component 3a receptor 1; comple . . . |
| ref\|NP_005290.1\| | G protein-coupled receptor 31 [Homo sapiens] |
| ref\|NP_005233.2\| | coagulation factor II (thrombin) receptor- . . . |
| ref\|NP_003546.1\| | olfactory receptor, family 1, subfamily G, . . . |
| ref\|XP_003671.3\| | coagulation factor II (thrombin) receptor- . . . |
| ref\|NP_112163.1\| | olfactory receptor, family 7, subfamily A, . . . |
| ref\|NP_039229.1\| | olfactory receptor, family 10, subfamily C . . . |
| ref\|NP_002539.1\| | olfactory receptor, family 1, subfamily D, . . . |
| ref\|XP_037263.1\| | similar to coagulation factor II (thrombin . . . |
| ref\|NP_067647.1\| | leucine-rich repeat-containing G protein-c . . . |
| ref\|NP_065103.1\| | inflammation-related G protein-coupled rec . . . |
| ref\|NP_055314.1\| | putative purinergic receptor [Homo sapiens . . . |
| ref\|NP_036509.1\| | olfactory receptor, family 7, subfamily C, . . . |
| ref\|NP_036507.1\| | olfactory receptor, family 52, subfamily A . . . |
| ref\|XP_035507.1\| | similar to NONE_RETURNED |
| | (H. sapiens) [Hom . . . |
| ref\|XP_004280.1\| | G protein-coupled receptor 31 [Homo sapiens] |
| ref\|NP_003545.1\| | olfactory receptor, family 1, subfamily E, . . . |
| ref\|XP_036497.1\| | olfactory receptor, family 1, subfamily F, . . . |
| ref\|NP_061844.1\| | super conserved receptor expressed in brai . . . |
| ref\|NP_063941.1\| | melanocortin 3 receptor [Homo sapiens] |
| ref\|NP_009091.1\| | olfactory receptor, family 2, subfamily H, . . . |
| ref\|XP_008678.2\| | olfactory receptor, family 1, subfamily D, . . . |
| ref\|NP_003543.1\| | olfactory receptor, family 1, subfamily D, . . . |
| ref\|XP_011731.3\| | similar to adrenergic, beta-3-, receptor ( . . . |
| ref\|XP_009545.1\| | melanocortin 3 receptor [Homo sapiens] |
| ref\|NP_076403.1\| | G protein-coupled receptor 86 [Homo sapiens] |
| ref\|XP_042200.1\| | G protein-coupled receptor 86 [Homo sapiens] |
| ref\|NP_004711.2\| | endothelial differentiation, lysophosphati . . . |
| ref\|NP_037440.1\| | platelet activating receptor homolog [Homo . . . |
| ref\|NP_005217.1\| | endothelial differentiation, sphingolipid . . . |
| ref\|NP_005295.1\| | G protein-coupled receptor 41 [Homo sapien . . . |
| ref\|NP_005296.1\| | G protein-coupled receptor 42 [Homo sapiens] |
| ref\|NP_115943.1\| | putative chemokine receptor [Homo sapiens] . . . |
| ref\|NP_055381.1\| | olfactory receptor, family 1, subfamily D, . . . |
| ref\|XP_042826.1\| | luteinizing hormone/choriogonadotropin rec . . . |
| ref\|XP_010797.3\| | luteinizing hormone/choriogonadotropin rec . . . |
| ref\|NP_000224.1\| | luteinizing hormone/choriogonadotropin rec . . . |

### EXAMPLE 14

### Riboreporter PDE Target Validation

Riboreporters are raised against various subclasses of PDE for target validation. For example riboreporters are raised against each of four PDE4 subtypes. The four subclasses of PDE4 are differently localized both between cells and with differ with respect to their intracellular distribution. This differential localization, together with the transcriptional regulation and post-translational modification, controls the cAMP level in cells in response to the cells' environment(Muller, Engels et al. 1996).

The cDNAs for four PDE4 subtype are cloned from human blood leukocyte cDNA library as described (Wang, Myers et al. 1997). Each subclass can be expressed as recombinant protein fused with His-tag in E. coli or insect cells (Richter, Hermsdorf et al. 2000) (Wang, Myers et al. 1997). The expressed proteins are purified through Ni⁺⁺ columns according to manufacturer's recommendation (Promega). Riboreporters against four subclass of PDE4 are raised as described above. The riboreporters are tested for their subclass specificity.

Tissue samples from different organs can be prepared, and the cell extract can be tested against a panel of PDE4 subclass-specific riboreporters to determine the protein level of each PDE in the organ. Thus, one can obtain more precise information about PDE4 levels relative to methods based on measuring the mRNA level (Bloom and Beavo 1996) (Obernolte, Ratzliff et al. 1997) (Nagaoka, Shirakawa et al. 1998).

Different classes of PDE (PDE1-11) are expressed tissue-specific manner and play different physiological roles(Conti 2000), and the subcellular localization of PDE regulates their activity. Accordingly, the riboreporter can be used to determine the subcellular localization of each PDE from fractionated cell extracts (Bolger, Erdogan et al. 1997), or in situ hybridization technique (Sirinarumitr, Paul et al. 1997).

### EXAMPLE 15

### Nucleotide Sequence and Activity of a cAMP-dependent PDE Riboreporter

The nucleotide sequence of a cAMP-dependent PDE riboreporter and cGMP-dependent riboreporter is presented below. Allosteric domains and cleavage site nucleotide are shown in bold font.

| | |
|---|---|
| cAMP-Hammerhead RNA seq: | (SEQ ID NO:34) |

5′-GGGC GAC CC UGA UGA GCC UGU GGA AAC AGA CGU GGC
ACA UGA CUA CGU CGA AAC GGU GAA AGC CGU AGG UUG CCC-
3′

| | |
|---|---|
| cAMP-Hammerhead RNA seq: | (SEQ ID NO:35) |

5′-GGGC GAC CC UGA UGA GCC CUG CGA UGC AGA AAG GUG
CUG ACG ACA CAU CGA AAC GGU GAA AGC CGU AGG UGG
CCC-3′

The cAMP and cGMP-dependent riboreporters were added to a solution containing various amounts of PDE and the corresponding cyclic nucleotide (cAMP or cGMP). The decreasing amount of the cyclic nucleotide corresponds to the increasing amount of PDE. These results demonstrate that the cyclic nucleotide-dependent riboreporters can be used to measure PDE activity.

### EXAMPLE 16

### High Throughput Screening (HTS) Assays Using cAMP-dependent PDE Riboreporters

A cAMP-dependent riboreporter can be used in HTS assays for PDEs (PDE1, PDE2, PDE3, PDE4, PDE7, PDE8, PDE10, and PDE11). Similarly, cGMP-dependent-riboreporters can be used in HTS assays for PDEs (PDE5, PDE9, PDE10, and PDE11). Representative cAMP-dependent and cGMP-dependent PDE riboreporters are shown in Example 15.

Each class of PDE can be isolated from human tissue (Ballard, Gingell et al. 1998), or expressed as recombinant proteins in various system (e.g. E coli, SF9 cells). Thus, the riboreporter can monitor the PDE activity in the presence and the absence of candidate drugs. For example, PDE and its substrate (i.e. cAMP and/or cGMP) are incubated at predetermined durations in a multiwell chamber (e.g. 96, 384 well) with various concentration of compounds for screening, and the reaction is terminated by changing the buffer conditions (e.g. addition of sufficient amount of EGTA, shifting buffer pH), or by separating enzyme and substrate (e.g. filteration). Next, the riboreporters are added to measure the altered concentration of the substrate, cAMP and cGMP. Alternatively, reengineered riboreporter (e.g. FRET, riboreporter beacon) as described previously can be added without terminating the PDE activity.

cAMP- or cGMP-dependent riboreporters can also be used to characterize the IC50 of the drug in vitro. A PDE assay is performed with serial dilutions of a compound of interest. Purified PDE or soluble extract from cells(Moreland, Goldstein et al. 1998) can be used for the assay. The assay can be performed as described above.

Alternatively, cAMP- or cGMP-dependent riboreporters are used to characterize the IC50 values of drug candidate in vitro, by analyzing cAMP- or cGMP synthesized by adenyl and guanyl cyclases. Adenylate and guanylate cyclase assays will be set up with series dilution of a compound of interest. Membrane fractions containing Adenylate and guanylate cyclases are used for the assay. The assay can be setup as described in the literature using ATP or GTP as the substrate.

### EXAMPLE 17

### Competitive Assay Using PDE Riboreporters

Riboreporters are generated that interact with the active sites of PDEs. PDE4 proteins are obtained as described above. The riboreporters are selected against PDE4 with negative selection in the presence of PDE4 complexed with subnanomolar inhibitor (Rolipram). Thus, the riboreporter requires an empty active site to recognize PDE4, and riboreporters compete for PDE binding with inhibitors.

The direct inhibition by the riboreporters can be tested using commerically available PDE assay kits (Amersham SPA assay kit for cAMP, Molecular Devices HEEP cAMP assay kit). In drug screening, the competition is performed with monitoring the signal from riboreporters in the presence of various inhibitors. Purified PDE or soluble cell extract from appropriate source (e.g. Wistar rat brain, (Andersson, Gemalmaz et al. 1999) ) is incubated with riboreporters (100 nM) in the presence and the absence of compounds in 10 mM Tris buffer pH7.5 containing 10 mM MgCl₂. The changes in the initial rate of each riboreporter response in the presence and the absence of the drug can be monitored in homologous system. Multiple PDEs can be tested against a same compound in the same well. This assay is expanded if desired to determine the tissue specific interaction of each class of PDE and any compounds.

### EXAMPLE 18

### Cell-based Assays Using Cyclic Nucleotide-dependent PDE Riboreporters

Riboreporters are used to monitor the cellular cAMP and cGMP level as response to the injection of drugs in tissue or rat cell lines. For example, strips of human corpus collasum (HCC) tissue or rat HCC cell lines (N1S1 and McA-RH7777 cells) can be incubated in the presence and absence of a drug against PDE5(Min, Kim et al. 2000) (Arora, de Groen et al. 1996), and the cGMP specific riboreporter can be used to measure the amount of cGMP in soluble extract from the tissue or cell sample as described above.

Alternatively, the cAMP, and cGMP-dependent riboreporters are incorporated into a reporter-gene plasmid as described above. This construct is introduced in cell lines by standard transfection (e.g. lipid-mediated transfection, calcium-phosphate co-precipitation, microinjection, electroporation, retroviral infection). The level of cGMP or camp in the cell is measured by the expression of the reporter gene.

### EXAMPLE 19

### Pharmacokinetics Studies Using Riboreporters

Riboreporters for drugs in preclinical and clinical trial for pharmacokinetics studies are prepared. A human serum sample with or without the administration of a drug or other therapeutic agent is prepared (Berzas Nevado, Rodriguez Flores et al. 2001). The riboreporter is added to the sample. The riboreporter is then detected, thereby measuring the drug concentration in the whole serum or extract from the serum.

The riboreporter for drugs can also be used to determine the drug distribution in an animal model system. For example, a drug can be administrated in animals (i.e. Sprague Dawley rats, New Zealand white rabbit) by IV or orally(Andersson, Gemalmaz et al. 1999) (Jeremy, Ballard et al. 1997). At various time intervals after drug administration, the animal is sacrificed. Various organs are tested for the drug distribution by in situ hybridization using the drug-dependent riboreporter. Alternatively, each organs/serum is prepared for pharmacokinetic studies as described above.

### EXAMPLE 20

### Cell-permeability Studies Using Riboreporters

Riboreporters against a test compound are used to test cell permeability of the compound. These riboreporters can be incorporated into a reporter gene construct, if desired, to make a drug-sensitive reporter gene system as described above. This construct is introduced in established cell lines (e.g. HELA cells, 293 cell, CHO cell). The cells are cultured in various concentrations of drug in media, and the expression of the reporter gene is monitored.

### EXAMPLE 21

### Class Specific PDE Assay

Riboreporters raised against catalytic domains of each class of PDE1-11 are prepared. We raise riboreporters against the catalytic domains of all PDEs. These riboreporters can be used for target validation as described above.

Alternatively, the riboreporters are used in competitive inhibition assays. Competitive riboreporters are used in vitro assays to screen compounds against multiple PDEs in multiplex assays, as described above.

### EXAMPLE 22

### Riboreporters Activated by Native ERK1 and Native ERK2 Enzymes

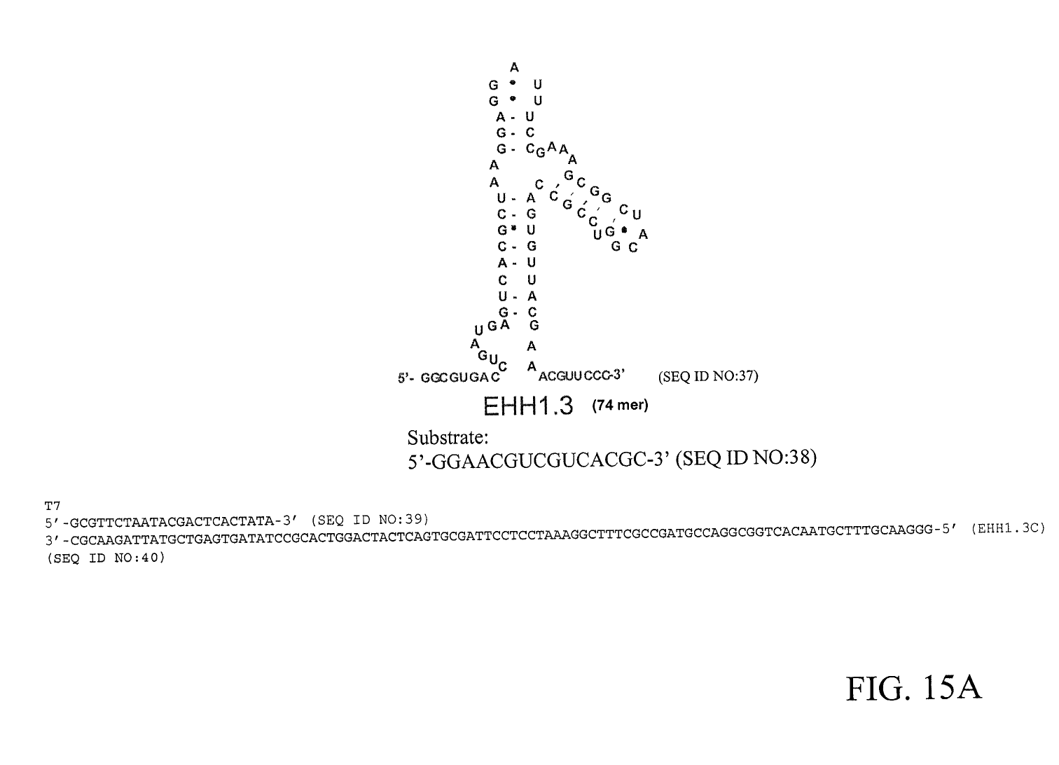

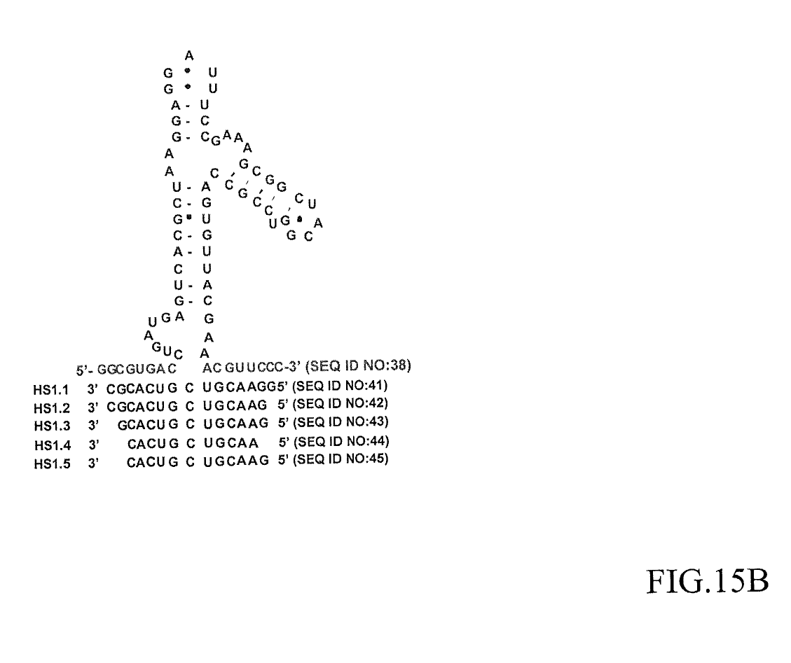

A 74 nucleotide hammerhead riboreporter that is activated by native ERK1 and native ERK2 enzymes the nucleotide sequence and regions of secondary structure shown in FIG. 15A (EHH1.3) (SEQ ID NO:37) along with a substrate (SEQ ID NO:38). A partially double-stranded DNA construct that can be used to express the EHH1.3 riboreporter is also shown (SEQ ID NO:39 and SEQ ID NO:40). The hammerhead riboreporter and additional substrates are shown in FIG. 15B as SEQ ID NOs: 41-45.

The time course of signal generation in the presence of nonphosphorylated ERK, phosphorylated ERK, and in the absence of protein is determined by measuring signal released over time by a radiolabeled riboreporter. Significant amounts signal, corresponding to cleavage of the riboreporter is observed over time only with the nonphosphorylated ERK. The signal obtained using the riboreporter decreases if the riboreporter is incubated with the enzyme in the presence of an ERK inhibitor. The decrease in riboreporter signal is dependent on the concentration of the inhibitor.

These results demonstrate that the riboreporter is specific for the non-phosphorylated form of the ERK.

### EXAMPLE 23

### Riboreporters Activated by ppERK

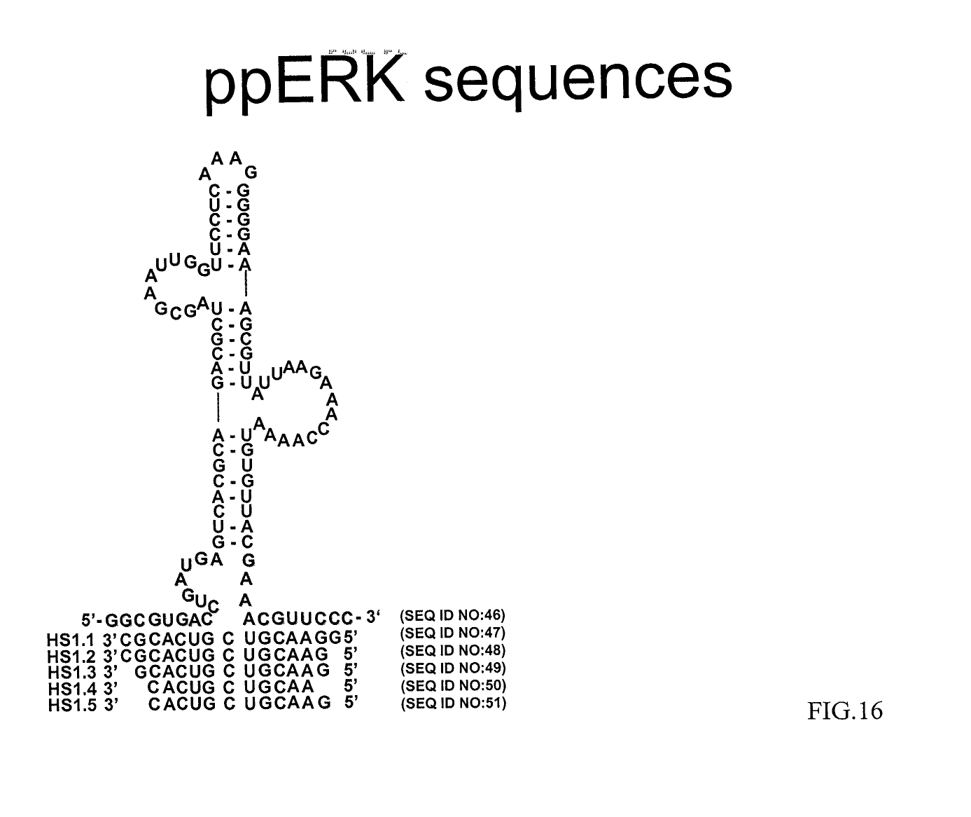

A riboreporter activated by ppERK is shown in FIG. 16 as SEQ ID NO:46 along with associated substrates (SEQ ID NOs:47-51). The riboreporter and substrate HS1.1 (SEQ ID NO:47) are allosterically activated by ppERK.

### EXAMPLE 24

### High Throughput Screening (HTS) Assays Using ppERK Riboprobes

The competitive inhibitor riboreporter can be used in a HTS assay. A riboreporter specific for phosphorylated ERK is used as a competitive inhibitor for ATP binding (Seiwert, Stines Nahreini et al. 2000). A competitive assay for compounds is established by incubating ERK with 10 nM riboreporter in the presence of various concentrations of the inhibitors in 10 mM Tris buffer pH7.5 containing 0.5 ug/ul tRNA and 10 mM MgCl₂. The reactions are terminated by addition of EDTA and the amount of reacted riboreporter is determined(figure).

In other embodiment, the riboreporters that are sensitive for the phosphorylation state of peptides (or protein substrates) can be used HTS assay for kinase activity. The riboreporters can be raised specific against the phosphorylation state of substrates or its peptides(see table). HTS assay can be performed using these substrate (Mansour, Candia et al. 1996). For example, MEK can be expressed as GST-tagged protein and purified by a standard method. The activity are measured at 30 C under standard reaction conditions of 20 mM HEPES (pH 7.4), 2 mM dithiothreitol, 0.01% Triton X-100, 10 mM MgCl₂, 0.1 mM ATP, and 1M His-tagged ERK2, at concentrations of MEK, 0.5 nM, in the presence of various concentration of compounds. After incubation, the reaction is terminated by absorbing MEK with GST-column. Phosphorylation of ERK2 was quantified by riboreporter. Alternatively the riboreporter can be used in western-blotting format (Bianchini, Radrizzani et al. 2001).

The riboreporter specific for phosphoERK (ppERK) described in Example 23 binds to ppERK, presumably in a competitive manner at the active site of the kinase enzyme, and inhibits the phosphorylation of the ppERK substrate by ppERK enzyme. Up to 200 nM riboreporter inhibits up to 80% of the ppERK phosphorylation of ERK substrate.

### EXAMPLE 24

### In vivo Assays Using Phosphorylation State-sensitive Riboreporters

The phosphorylation state sensitive ppERK riboreporter is used to determine drug efficacy in vivo (e.g. tissue and cell culture). For example, T84 cells on glass coverslips are incubated in the presence and the absence of the MEK kinase inhibitor, and cells are fixed by 4% paraformaldehyde and permeabilized using 0.3% Triton X-100. The slides are incubated with FRET riboreporter (Bianchini, Radrizzani et al. 2001). The localization of phosphorylated substrate can be observed using a confocal microscope.

Alternatively, phosphorylation-state-sensitive riboreporter are incorporated into reporter-gene constructs discussed above. These constructs are introduced into cells, and phosphorylation of the substrates is monitored.

Riboreporters made of nuclease resistant forms of allosteric-hammerhead, -ligase or -hairpin ribozymes are transfected into mammalian cells using standard lipofectarnine reagents or liposomal solutions known to effect internalization an cellular uptake of polynucleotides.

If desired, the riboreporter allosteric-hammerhead, -ligase or -hairpin ribozymes or aptamer beacons or signaling apatamers are attached to polypeptides such as tar or antennapoedia or the like and are transported into mammalian cells by methods known in the art.

The activity of riboreporter systems is followed by changes in fluorescence if the Riboreporter is fluorescently tagged, or by changes in size as determined by RT-PCR of product and substrate forms of the allosteric ribozyme.

### EXAMPLE 25

### High Throughput Screening (HTS) Assay Using G Protein Riboreporters

Activated Gα-protein dependent riboreporters are used in vitro to test the efficacy of the agonists and inverse agonists for any GPCR. Giα-protein cDNA can be obtained (Guthrie cDNA resource Center) and expressed in E coli as His-tagged protein(Lee, Linder et al. 1994). GDP or GTP-γS are added during the purification to avoid degradation. GDP-complex Giα-protein and GTP-complex Giα-protein are used to raise riboreporters.

GTP-complex Giα-protein(activated-Giα-protein)-dependent riboreporter are tested in a binding assay to screen the selected riboreporters with desirable properties. For example, membrane fraction of C6 gloom cells in 12-well plates are loaded for 16 h in the presence and the absence of adrenaline. Cell lysate can be tested for activated Giα-protein-dependent riboreporter response. Alternatively, the competitive binding for adenylyl cyclase is tested following the inhibition of adenylyl cyclase activity by activated Giα-protein in the presence of riboreporters by a modified assay based on a previously described assay(Burt, Sauté et al. 1998).

Adenylyl cyclase activity is measured as described in the presence and the absence of riboreporters (Kozasa and Gilman 1995). Cell membranes from HeLa cells transfected with human cloned 5-HT_{1A} receptors resuspended in buffer are used to screen compounds. The membranes are incubated with 30 M GDP and decreasing concentrations of test drugs (from 100 pM to 0.1 nM) or decreasing concentrations of 5-HT, from 100 M to 0.1 nM (reference curve) for 20 min at 30 C in a final volume of about 0.5 mL (Stanton and Beer 1997). Then samples are added with GTPS and the riboreporters and then incubated for a further 30 min at 30 C.

Alternatively, riboreporters are raised that depend on the presence of Gβ-protein uncomplexed with Gα-protein. There are four known classes of Gβ-protein. Gβ-protein complex with Gγ-protein can also play important roles in the signal transduction. After dissociation from alpha subunit, this complex is known to regulate various target protein, such as GRKs, Raf kinase, adenylyl cyclase, PLCs and ion channels. Producing block the complex for activating their effectors.

### EXAMPLE 26

### Cell-based Assays Using GPCR Riboreporters

GPCR riboreporters are used in cell-based assay using modifications of previously described GPCR assays (Hun, Ellington et al. 2001). For example, β-adrenergic receptor can be expressed in CHO cells. CHO cells are grown in multiwell tissue culture plates in Dulbecco's modified Eagle media (DMEM) with 10% fetal bovine serum. On the day of assay, medium was replaced with 0.2 mL treatment medium containing DMEM media containing 250 M IBMX (isobutyl-1-methylxantine) plus 1 mM ascorbic acid with test compound dissolved in DMSO. Test compounds are added at a desired concentration range (e.g. 10⁻⁹ to 10⁻⁴ M). Isoproterenol (10⁻⁵ M) is used as an internal standard for comparison of activity. Cells were incubated at 37 C on a rocker for 15-30 min. Then cells are lysed and the level of the activated Gα-protein is measured by the riboreporter. The antagonist can be screened in the same format in the presence of the known agonist by detecting the decreasing amount of the activated Gα-protein.

Alternatively, the agonism and the antagonism of compounds for specific or general GPCR is measured using the riboreporter. For example, evaluation of compounds for ₁-antagonism can be performed using Sprague Dawley rats. The aorta from animal is isolated and freed of adhering connective tissue. Desmethylimipramine (0.1 M) and corticosterone (1 M) to block neuronal and extraneuronal uptake of noradrenaline, (±)-propranolol (1M) to block β-receptors, and yohimbine (0.1 M) to block ₂-receptors can be added. Aortic strips are incubated with various concentration of compounds in the presence and the absence of 10 M noradrenaline. Then the cell extract can be prepared and the activated Giα-protein level in the cell can be measured with the riboreporters (Barlocco, Cignarella et al. 2001).

### EXAMPLE 27

### Multiplex Assays Using IMPDH Riboreporters

The IMPDH type 1 polypeptide and IMPDH type 2 polypeptides share 84% homology. An aptamer is isolated based on its ability to bind the polypeptide IMPDH-1 (“IMPDH-1 Aptamer”) is prepared, as is an aptamer that based on the ability to bind the polypeptide IMPDH-2 (“IMPDH-2 Aptamer”). Activity of the aptamers is determined in the presence of IMPDH-1, in the presence of IMPDH-2, or in the absence of exogenous protein. Little binding is observed of either IMPDH-1 Aptamer or IMPDH-2 aptamer is observed in the absence of exogenous protein. While IMPDH-1 binds both IMPDH-1 and IMPDH-2 polypeptides, the IMPDH-1 aptamer binds with higher affinity to IMDH-1 polypeptide. IMPDH-2 shows significant binding to IMPDH-2 polypeptide but shows no significant binding to IMPDH-1 polypeptide.

### EXAMPLE 28

### In vitro Assay for GPCR Activation by Following Liberation of Gα-protein with Gα-protein Dependent Riboreporters.

Riboreporters raised against Gα-protein subunits are used to test the efficacy of agonists and inverse agonists for any GPCR in vitro using activated G. For example, Giα-protien cDNA can be obtained (Guthrie cDNA resource Center) and expressed in E coli as His-tagged protein(Lee, Linder et al. 1994). GDP or GTP-γS are added during the purification to avoid degradation. GDP-complex Giα-protein and GTP-complex Giα-protein are used to raise riboreporters. GTP-complex Giα-protein(activated-Giα-protein)-dependent riboreporter can be tested for the binding assay to screen the selected riboreporters with desirable properties. For example, membrane fraction of C6 glioma cells in 12-well plates were loaded for 16 h in the presence and the absence of adrenaline. Cell lysate can be tested for activated Giα-protein-dependent riboreporter response. Alternatively, the competitive binding for adenylyl cyclase can be tested following the inhibition of adenylyl cyclase activity by activated Giα-protein in the presence of riboreporters by a modified assay based on a previously described assay(Burt, Sautel et al. 1998).

Adenylyl cyclase activity can be measured as described in the presence and the absence of riboreporters (Kozasa and Gilman 1995). Cell membranes from HeLa cells transfected with human cloned 5-HT_{1A} receptors resuspended in buffer can be used to screen compounds. The membranes are incubated with 30 mM GDP and decreasing concentrations of test drugs or decreasing concentrations of 5-HT, from 100 M to 0.1 nM (reference curve) for 20 min at 30 C in a final volume of about 0.5 mL (Stanton and Beer 1997). Then samples are added with GTPS and the riboreporters and then incubated for a further 30 min at 30° C.

Riboreporters can also be raised that are depend on the presence of Gβ-protein uncomplexed with Gα-protein. There are four known class of Gβ-protein. Gβ-protein complex with Gγ-protein can also play important roles in the signal transduction. After dissociation from alpha subunit, this complex is known to regulate various target protein, such as GRKs, Raf kinase, adenylyl cyclase, PLCs and ion channels. Phosducin block the complex for activating their effectors.

### EXAMPLE 29

### Cell-based Assay GPCR Assay Following Gα-activation.

Riboreporters are used in cell-based assay in similar to the assay described previously(Hu, Ellingboe et al. 2001). For example, βP-adrenergic receptor can be expressed in CHO cells. CHO cells are grown in multiwell tissue culture plates in Dulbecco's modified Eagle media (DMEM) with 10% fetal bovine serum. On the day of assay, medium is replaced with 0.2 mL treatment medium containing DMEM media containing 250 mM IBMX (isobutyl-1-methylxantine) plus 1 mM ascorbic acid with test compound dissolved in DMSO. Test compounds are added at a desired concentration range (e.g. 10⁻⁹ to 10⁻⁴ M). Isoproteren 10⁻⁵ M) is used as an internal standard for comparison of activity. Cells were incubated at 37° C. on a rocker for 15-30 min. Then cells are lysed and the level of the activated Gα-protein can be measured by the riboreporter. The antagonist can be screening in the same format in the presence of the known agonist by detecting the decreasing amount of the activated Gα-protein.

Alternatively, the agonism and the antagonism of compounds for specific or general GPCR can be measured using the riboreporter. For example, evaluation of compounds for antagonism can be performed using Sprague Dawley rats. The aorta from animal is isolated, freed of adhering connective tissue. Desmethylimipramine (0.1 M) and corticosterone (1 M) to block neuronal and extraneuronal uptake of noradrenaline, (±)-propranolol (1M) to block β-receptors, and yohimbine (0.1 mM) to block ₂-receptors can be added. Aortic strips can be incubated with various concentration of compounds in the presence and the absence of 10 mM noradrenaline. Then the cell extract can be prepared and the activated Giα-protein level in the cell can be measured with the riboreporters (Barlocco, Cignarella et al. 2001).

### EXAMPLE 30

### Multiplex Assay for Gα-proteins

Riboreporters are raised whose activity is dependent on multiple activated G-proteins. Human G-protein cDNAs are obtained from publicly available databases or are cloned by RT-PCR from polka-RNA pool of appropriate source. They can be expressed as described above and use to select riboreporters. The readouts for multiplex assay system are discussed above.

The following references provided include additional information, the entirety of which is incorporated herein by reference.

References

Andersson, K. E., H. Gemalmaz, et al. (1999). “The effect of sildenafil on apomorphine-evoked increases in intracavernous pressure in the awake rat.” J Urol 161(5): 1707-12.

Arora, A. S., P. C. de Groen, et al. (1996). “Hepatocellular carcinoma cells resist necrosis during anoxia by preventing phospholipase-mediated calpain activation.” J Cell Physiol 167(3): 434-42.

Asher, et al., PCT 98/08974 (1996)

Ballard, S. A., C. J. Gingell, et al. (1998). “Effects of sildenafil on the relaxation of human corpus cavernosum tissue in vitro and on the activities of cyclic nucleotide phosphodiesterase isozymes.” J Urol 159(6): 2164-71.

Barlocco, D., G. Cignarella, et al. (2001). “Phenylpiperazinylalkylamino substituted pyridazinones as potent alpha(1) adrenoceptor antagonists.” J Med Chem 44(15): 2403-10.

Basararsky, T. et al., “Overview of a Microarray Scanner: Design Essentials for an Integrated Acquisition and Analysis Platform”, in Microarray Biochip Technology, ed. by M. Schena, Eaton Publishing Co., 2000, Natick, Mass., pp. 265-284.

Beaudet L. et al., “Homogeneous Assays for Single-Nucleotide Polymorphism Typing Using AlphaScreen”, Genome Res. 11:600 (2001).

Bell, et al., J. Biol. Chem. 273: 14309 (1998).

Berzal-Herranz A; Joseph S; Burke J M. In vitro selection of active hairpin ribozymes by sequential RNA-catalyzed cleavage and ligation reactions. Genes and Development, Jan. 6, 1992(1):129-34.

Berzas Nevado, J. J., J. Rodriguez Flores, et al. (2001). “Micellar electrokinetic capillary chromatography for the determination of Viagra and its metabolite (UK-103,320) in human serum.” Electrophoresis 22(10): 2004-9.

Bianchini, M., M. Radrizzani, et al. (2001). “Specific oligobodies against ERK-2 that recognize both the native and the denatured state of the protein.” J Immunol Methods 252(1-2): 191-7.

Bloom, T. J. and J. A. Beavo (1996). “Identification and tissue-specific expression of PDE7 phosphodiesterase splice variants.” Proc Natl Acad Sci USA 93(24): 14188-92.

Bock, et al., Nature, 355(6360): 564-6 (1992)

Bolger, G. B., S. Erdogan, et al. (1997). “Characterization of five different proteins produced by alternatively spliced mRNAs from the human cAMP-specific phosphodiesterase PDE4D gene.” Biochem J 328(Pt 2): 539-48.

Breaker,. PCT WO 98/43993 (1998).

Breaker, PCT WO 98/27104 (1998)

Bruchez, M. et al., “Semiconductor Nanocrystals as Fluorescent labels”, Science 281, 2013 (1998).

Burt, A. R., M. Sautel, et al. (1998). “Agonist occupation of an alpha2A-adrenoreceptor-Gi1 alpha fusion protein results in activation of both receptor-linked and endogenous Gi proteins. Comparisons of their contributions to GTPase activity and signal transduction and analysis of receptor-G protein activation stoichiometry.” J Biol Chem 273(17): 10367-75.

Caceci, et al., Byte 9: 340-362 (1984).

Chan, V. et al., Effect of Hydrophobocity and Electrostatics on Adsorption and Surface Diffusion of DNA Oligonucleotides at Liquid/Solid Interfaces”, J. Colloid and Interface Sci. 203:197 (1998).

Chan, W. C. W and Nie, S., “Quantum Dot Bioconjugates for Ultrasensitive Nonisotopic Detection”, Science 281: 2016 (1998)

Coleman, D. E. and S. R. Sprang (1998). “Crystal structures of the G protein Gi alpha 1 complexed with GDP and Mg2+: a crystallographic titration experiment.” Biochemistry 37(41): 14376-85.

Ciesiolka et al., Methods in Enzymology 267: 3 15-335 (1996)

Conti, M. (2000). “Phosphodiesterases and cyclic nucleotide signaling in endocrine cells.” Mol Endocrinol 14(9): 1317-27.

Cook, N. D. et al., “SPA: a revolutionary new technique for drug screening”, Pharmaceutical Manufacturing International, 1992, pp. 49-53.

Cooper, et al., Biochemistry 29: 926 1-9268 (1990).

Cotten, et al., Nuc. Acid Res. 19: 2629-2635 (1991).

Cox J C; Rudolph P; Ellington A D. Automated RNA selection. Biotechnology Progress, Nov.-Dec. 14, 1998(6):845-50.

DeRisi et al., Science 278: 680-686 (1997).

Ellington and Szostak, Nature 346(6287): 818-822 (1990)

Fehrentz, J. A. et al., “Biochem. Biophys. Res. Commun. 188:865 (1992)

Froehler, et al., Nucleic Acids Research 14: 53 99-5467 (1986a).

Froehler, Tet. Lett. 27: 5575-5578 (1986b).

Franke, B., J. W. Akkerman, et al. (1997). “Rapid Ca2+-mediated activation of Rap1 in human platelets.” Embo J 16(2): 252-9.

Gold, et al., Annual Review of Biochemistry 64: 763-797 (1995).

Gold, et al., Proc. Natl. Acad. Sci. 94: 59-64 (1997). 10 Griffiths, et al. EMBO. J 13: 3245-3260 (1994).

Goueli, S. A., K. Hsiao, et al. (2001). “Assaying activity of individual protein kinases in crude tissue or cellular extracts.” Methods Enzymol 333: 16-27.

Green R; Szostak J W. Selection of a ribozyme that functions as a superior template in a self-copying reaction. Science, Dec. 18, 1992, 258(5090):1910-15

Guatelli, et al., Proc. Natl. Acad. Sci. 87: 1874 (1990).

Hamaguchi, N. et al., “Aptamer Beacons for the Direct Detection of Proteins”, Anal. Biochem. 294: 126 (2001)

Han, M. et al., “Quantum-dot-tagged microbeads for multiplexed optical coding of biomolecules”, Nature Biotechnol. 19, 631 (2001).

Heath, R. et al., “cAMP [¹²⁵I] scintillation proximity assay (SPA)—a homogeneous radioimmunoassay for cAMP”, in Cell Signalling: Experimental Strategies, ed. Reid, E., Cook, G. M. W., and Luzio, J. R., 1992, pp. 193-194.

Heller and Morrison, in Rapid Detection and Identification of Infections Agents, Academic ress, Inc., San Diego, Calif., pages 245-256 (1985).

Heller, R. A. et al., “Gene Chips and microarrays: applications in disease profiles, drug target discovery, drug action and toxicity”, in DNA Microarrays: A Practical Approach, ed. by M. Schena, Oxford University Press, 1999, Oxford, pp. 187-202.

Hirose, et al., Tet. Lett. 28: 2449 (1978).

Hobbs, et al.,Biochemistry 12: 5138-5145 (1973).

Hu, B., J. Ellingboe, et al. (2001). “(4-Piperidin-1-yl)phenyl amides: potent and selective human beta(3) agonists.” J Med Chem 44(9): 1456-66.

Jameson, D. M. and Sawyer, W. H., “Fluorescence Anisotropy Applied to Biomolecular Interactions”, Methods in Enzymology 246:283 (1995).

Jameson, D. M. and Seifried, S. E., “Quantificatin of Protein-Protein Interactions Using Fluorescence Polarization”, METHODS 19:222 (1999).

Jellinek, et al., Biochemistry 34: 11363-11372 (1995).

Jenne A; Hartig J S; Piganeau N; Tauer A; Samarsky D A; Green M R; Davies J; Famulok M. Rapid identification and characterization of hammerhead-ribozymne inhibitors using fluorescence-based technology. Nature Biotechnology, Jan. 19, 2001(1):56-61.

Jeremy, J. Y., S. A. Ballard, et al. (1997). “Effects of sildenafil, a type-5 cGMP phosphodiesterase inhibitor, and papaverine on cyclic GMP and cyclic AMP levels in the rabbit corpus cavernosum in vitro.” Br J Urol 79(6): 958-63.

Jhaveri, et al. J. Am. Chem. Soc. 122: 2469-2473 (2000).

Jenison, et al. Science, 263(5 152): 1425-9 (1994).

Jolley, M., “Fluorescence Polarization: an analytical tool for immunoassay and drug discovery”, Comb. Chem. High Throughput Screen. 2(4):177 (1999).

Joyce, in Molecular Biology of RNA: UCLA Symposia on Molecular and Cellular Biology, T. R.Cech (ed.), Liss, N.Y., 1989, pp. 361-371.

Koizumi, M., G. A. Soukup, et al. (1999). “Allosteric selection of ribozymes that respond to the second messengers cGMP and cAMP.” Nat Struct Biol 6(11): 1062-71.

Koizumi, M., J. N. Kerr, et al. (1999). “Allosteric ribozymes sensitive to the second messengers cAMP and cGMP.” Nucleic Acids Symp Ser 42: 275-6.

Kozasa, T. and A. G. Gilman (1995). “Purification of recombinant G proteins from Sf9 cells by hexahistidine tagging of associated subunits. Characterization of alpha 12 and inhibition of adenylyl cyclase by alpha z.” J Biol Chem 270(4): 1734-41.

Kraus, et al., Journal of Immunology 160: 5209-52 12 (1998)

Krishnan, et al., Science 282(53 88): 484-7 (1998).

Kwoh, et al., Proc. Natl. Acad. Sci. 86: 1173 (1989).

Kubik, et al., Nucleic Acids Res. 22(13): 2619-26 (1994).

Lakowicz, J. R. and Berndt, K., “Lifetime-selective fluorescence imaging using an rf phase-sensitive camera”, Rev. Sci. Instr. 62(7):1727 (1991).

Lakowicz, J. R., Principles of Fluorescence Spectroscopy, Second Edition, 1999. Kluwer Academic/Plenum Publishers, New York.

Lee, E., M. E. Linder, et al. (1994). “Expression of G-protein alpha subunits in Escherichia coli.” Methods Enzymol 237: 146-64.

Li, Y., P. M. Stermweis, et al. (1998). “Sites for Galpha binding on the G protein beta subunit overlap with sites for regulation of phospholipase Cbeta and adenylyl cyclase.” J Biol Chem 273(26): 16265-72.

Lin, et al. NucleicAcids Research 22: 5229-5234 (1994).

Liu, X. and Tan, W., “A fiberoptic evanescent wave DNA biosensor based on novel molecular beacons”, Anal. Chem. 71:5054 (1999).

Lockhart, et al., Science, 274(5287): 6 10-4 (1996).

Mace, M. L. et al., “Novel Microarray Printing and Detection Technologies”, in Microarray Biochip Technology, ed. by M. Schena, Eaton Publishing Co., 2000, Natick, Mass., pp. 39-64.

Morrison, in Nonisotopic DNA Probe Techniques, Kricka, ed., Academic Press, Inc., San Diego, Calif., chapter 13 (1992).

Mansour, S. J., J. M. Candia, et al. (1996). “Interdependent domains controlling the enzymatic activity of mitogen-activated protein kinase kinase 1. ” Biochemistry 35(48): 15529-36.

Min, K., N. N. Kim, et al. (2000). “Sildenafil augments pelvic nerve-mediated female genital sexual arousal in the anesthetized rabbit.” Int J Impot Res 12 Suppl 3: S32-9.

Moreland, R. B., I. Goldstein, et al. (1998). “Sildenafil, a novel inhibitor of phosphodiesterase type 5 in human corpus cavernosum smooth muscle cells.” Life Sci 62(20): 309-18.

Morrison, et al., Anal. Biochem 183: 23 1-244 (1989).

Muller, T., P. Engels, et al. (1996). “Subtypes of the type 4 cAMP phosphodiesterases: structure, regulation and selective inhibition.” Trends Pharmacol Sci 17(8): 294-8.

Nagaoka, T., T. Shirakawa, et al. (1998). “Cyclic nucleotide phosphodiesterase 3 expression in vivo: evidence for tissue-specific expression of phosphodiesterase 3A or 3B mRNA and activity in the aorta and adipose tissue of atherosclerosis-prone insulin-resistant rats.” Diabetes 47(7): 1135-44.

Obata, T., M. B. Yaffe, et al. (2000). “Peptide and protein library screening defines optimal substrate motifs for AKT/PKB.” J Biol Chem 275(46): 36108-15.

Obernolte, R., J. Ratzliff, et al. (1997). “Multiple splice variants of phosphodiesterase PDE4C cloned from human lung and testis.” Biochim Biophys Acta 1353(3): 287-97.

O'Shaughnessy, D. J., “Hydrazido-derivatized supports in affinity chromatography”, J. Chromatography, 510:13 (1990).

Owicki, J. C. et al., “Application of Fluorescence Polarization Assays in High-Throughput Screening”, Genetic Engineering News 17(19), 1997.

Pagratis, et al., Nat Biotechnol 15: 68-73 (1997).

Pieken, et al., Science 253: 314-3 17 (1991).

Pillai, R., K. Kytle, et al. (1993). “Use of a yeast expression system for the isolation and analysis of drug-resistant mutants of a mammalian phosphodiesterase.” Proc Natl Acad Sci USA 90(24): 11970-4.

Potyrailo, et al. Anal Chem, 70(16): 34 19-25 (1998).

Proudnikov, D. and Mirzabekov, A., “Chemical Methods of DNA and RNA fluorescent labeling”, Nucleic Acids Res. 24(22):4535 (1996).

Richter, W., T. Hermsdorf, et al. (2000). “Refolding, purification, and characterization of human recombinant PDE4A constructs expressed in Escherichia coli.” Protein Expr Purif 19(3): 375-83.

Robertson, D. and Davidson, N., “Covalent coupling of ribonucleic acid to agarose”, Biochemistry 11(4):533 (1972).

Robertson D L; Joyce G F. Selection in vitro of an RNA enzyme that specifically cleaves single-stranded DNA. Nature, Mar. 29, 1990, 344(6265):467-8.

Robertson, M. P. and Ellington, A. D., “Design and optimization of effector-activated ribozyme ligases”, Nucleic Acids Res. 28(8):1751 (2000).

Ruhn, P. F. et al., “Development of dihydrazide-activated silica supports for high-performance affinity chromatography”, J. Chromatography A 669:9 (1994).

Saiki, et al., Science 230: 1350-54 (1985).

Saiki, et al., Science 239: 487-49 1 (1988).

Sako, Y. et al., “Single-molecule imaging of EGFR signaling on the surface of living cells”, Nature Cell Bio. 2:168 (2000).

Schena, M. and Davis, R. W, “Technology Standards for Microarray Research”, in Microarray Biochip Technology, ed. by M. Schena, Eaton Publishing Co., 2000, Natick, Mass., pp1-18.

Schermer, M. J., “Confocal scanning microscopy in microarray detection”, in DNA Microarrays: A Practical Approach, ed. by M. Schena, Oxford University Press, 1999, Oxford, pp. 17-42.

Schluep, T. and Cooney, C. L., “Immobilization of oligonucleotides on a large pore support for plasmid purification by triplex affinity interaction”, Bioseparation 7:317 (1999).

Seelig B; Jaschke A. A small catalytic RNA motif with Diels-Alderase activity. Chemistry and Biology, Mar. 6, 1999(3):167-76.

Seetherman, S., et al., “Immobilized RNA switches for the analysis of complex and biological mixtures”, Nature Biotech. 19:336 (2001).

Serivce, “Coming soon: the pocket DNA sequencer.” Science 282, 399-401

Seiwert, S. D., T. Stines Nahreini, et al. (2000). “RNA aptamers as pathway-specific MAP kinase inhibitors.” Chem Biol 7(11): 833-43.

Simone, et al. Trends Genet 14(7): 272-6 (1998).

Singh, K. K. et al., “Rapid kinetic characterization of hammerhead ribozymes by real-time monitoring of fluorescence resonance energy transfer (FRET)”, RNA 5:1348 (1999).

Singh, K. K., et al., “Fluorescence Polarization for Monitoring Ribozyme Reactions in Real Time”, BioTechniques 29:344 (2000).

Sirinarumitr, T., P. S. Paul, et al. (1997). “Rapid in situ hybridization technique for the detection of ribonucleic acids in tissues using radiolabelled and fluorescein-labelled riboprobes.” Mol Cell Probes 11(4): 273-80.

Sokol, et al., Proc. Natl. Acad. Sci. 95(20): 11538-43 (1998).

Sood, et al., Nuci. Acid Res. 4:2557 (1977).

Soukup, et al. Journal of Molecular Biology 298: 623-632 (2000). 15 Sproat, et al., Nuci. Acid Res. 19:733-.738 (1991).

Soukup, G., et al., “Generating new ligand-binding RNAs by affinity maturation and disintegration of allosteric ribozymes”, RNA 7:524 (2001).

Stanton, M., Hamaguchi, N., and Ellington, A. Poster Presentation at the National Cancer Institute Innovative Molecular Analysis Technologies P1 Meeting, July 6-8, Chantilly, Va. (2000)

Stanton, J. A. and M. S. Beer (1997). “Characterisation of a cloned human 5-HT1A receptor cell line using [35S]GTP gamma S binding.” Eur J Pharmacol 320(2-3): 267-75.

Tasset, et al. JMol Biol, 272(5): 688-98 (1997).

Truskey, G. A. et al., “Total internal reflection fluorescence microscopy (TIRFM): Topographical mapping of relative cell/substratum separation distances”, J. Cell Science 103:491 (1992).

Tuerk and Gold, Science 249: 505(1990)

Taylor, S. J., R. J. Resnick, et al. (2001). “Nonradioactive determination of Ras-GTP levels using activated ras interaction assay.” Methods Enzymol 333: 333-42.

Tyagi, et al., Nature Biotechnology 14: 303-308 (1996),.

Walter, N. G. and Burke, J. M., “Real-time monitoring of hairpin ribozyme kinetics through base-specific quenching of fluorescein-labeled substrates”, RNA 3:392 (1997).

Walter, N. G. et al., “Tertiary structure formation in the hairpin ribozyme monitored by fluorescence resonance energy transfer”, The EMBO Journal 17(8):2378 (1998).

Wang, P., J. G. Myers, et al. (1997). “Expression, purification, and characterization of human cAMP-specific phosphodiesterase (PDE4) subtypes A, B, C, and D.” Biochem Biophys Res Commun 234(2): 320-4.

Wenzel-Seifert, K. and R. Seifert (2000). “Molecular analysis of beta(2)-adrenoceptor coupling to G(s)-, G(i)-, and G(q)-proteins.” Mol Pharmacol 58(5): 954-66.

Williams K P; Imahori H; Fujimoto D N; Inoue T. Selection of novel forms of a functional domain within the Tetrahymena ribozyme. Nucleic Acids Research, Jun. 11, 1994 22(11):2003-9.

Wilson C; Szostak J W. In vitro evolution of a self-alkylating ribozyme. Nature, Apr. 27, 1995 374(6525):777-82.

Wu, P. and Brand, L., “Resonance Energy Transfer: Methods and Applications”, Anal. Biochem. 218:1 (1994)

Wu, T. P. et al., “A fluorescent-labeling method for sequencing small RNA on polyacrylamide gel”, Nucleic Acids Res. 24(17):3472 (1996).

Zammatteo, N. et al., “Comparison between Different Strategies of Covalent Attachment of DNA to Glass Surfaces to Build DNA Microarrays”, Anal. Biochem. 280:143 (2000).

Zhang B; Cech T R. Peptide bond formation by in vitro selected ribozymes. Nature, Nov. 6, 1997, 390(6655):96-100.

Variations, modifications, and other implementations of what is described herein will occur to those of ordinary skill in the art without departing from the spirit and scope as claimed. Accordingly, the invention is to be defined not by the preceding illustrative description but instead by the spirit and scope of the following claims.

1. A nucleic acid sensor molecule, the nucleic acid sensor molecule comprising
a target molecule activation site, the target molecule activation site comprising
a structure that recognizes a target molecule and
an optical signaling unit, wherein said optical signaling unit includes at least one nucleotide coupled to a signaling moiety; and wherein said signaling moiety changes its optical properties upon allosteric modulation of said nucleic acid sensor molecule following recognition of said target molecule.

2. The nucleic acid sensor molecule of claim 1, wherein said optical signaling unit comprises a first nucleotide coupled to a first signaling moiety and a second nucleotide coupled a second signaling moiety, and wherein said first and second signaling moieties change proximity to each other upon recognition of said target molecule by said target activation site.

3. The nucleic acid sensor molecule of claim 2, wherein said first and second signaling moieties comprise a fluorescent label and a fluorescent quencher, and recognition by said nucleic acid sensor of said target molecule results in an increase in detectable fluorescence of said fluorescent label.

4. The nucleic acid sensor molecule of claim 2, wherein said first signaling moiety and said second signaling moiety comprise fluorescent energy transfer (FRET) donor and acceptor groups, and recognition by said nucleic acid sensor molecule of said target molecule results in a change in distance between said donor and acceptor groups, thereby changing optical properties of said molecule.

5. The nucleic acid sensor molecule of claim 1, wherein said optical signaling unit consists essentially of a first signaling moiety, wherein said first signaling moiety changes conformation upon recognition by said target molecule of said target activation site, thereby resulting in a detectable optical signal.

6. The nucleic acid sensor molecule of claim 1, wherein sad nucleic acid sensor molecule includes at least one modified nucleic acid.

7. The nucleic acid sensor molecule of claim 1, wherein said nucleic acid sensor molecule is RNA.

8. The nucleic acid sensor molecule of claim 1, wherein said nucleic acid sensor molecule is DNA.

9. The nucleic acid sensor molecule of claim 4, wherein said nucleic acid sensor molecule is RNA.

10. The nucleic acid sensor molecule of claim 1, wherein said target molecule is a polypeptide.

11. A method of detecting a target molecule, the method comprising
providing a nucleic acid sensor molecule, the molecule comprising a target molecule activation site, the target molecule activation site comprising a structure that recognizes a target molecule and an optical signaling unit, wherein said optical signaling unit includes at least one nucleotide coupled to a signaling moiety; and wherein said signaling moiety changing its optical properties upon allosteric modulation of said nucleic acid sensor molecule;
contacting said nucleic acid sensor molecule with a sample known to contain or suspected of containing a target molecule; and
detecting said light signal, wherein said light signal indicates the presence of said target molecule in said population.

12. The method of claim 11, wherein said optical signaling unit comprises a first nucleotide coupled to a first signaling moiety and a second nucleotide coupled a second signaling moiety, and wherein said first and second signaling moieties change proximity to each other upon recognition of said target molecule by said target activation site.

13. The method of claim 12, wherein said first and second signaling moiety comprise a fluorescent label and a fluorescent quencher, and recognition by said nucleic acid sensor of said target molecule results in an increase in detectable fluorescence of said fluorescent label.

14. The method of claim 11, wherein said first signaling moiety and said second signaling moiety comprise fluorescent energy transfer (FRET) donor and acceptor groups, and recognition by said nucleic acid sensor molecule of said target molecule results in a change in distance between said donor and acceptor groups, thereby changing optical properties of said molecule.

15. The method of claim 11, wherein said optical signaling unit consists essentially of a first signaling moiety, wherein said first signaling moiety changes conformation upon recognition of said target molecule by said target activation site, thereby resulting in a detectable optical signal.

16. The method of claim 11, wherein said target molecule is associated with a pathological condition or genetic alteration.

17. The method of claim 11, wherein a plurality of nucleic acid sensor molecules is provided

18. The method of claim 17, wherein a plurality of target molecules are detected.

19. A diagnostic profile produced by the method of claim 18.

20. The diagnostic profile of claim 19, wherein said diagnostic profile is correlated with a wild-type state, a pathological condition, or a genetic alteration.

21. A method identifying a nucleic acid sensor molecule, the method comprising
providing a population of oligonucleotides, wherein said population comprises oligonucleotides comprising a first region comprising a random nucleotide sequence;
contacting said population with a target molecule; and
identifying an oligonucleotide in said population that changes conformation upon recognizing said target molecule.

22. The method of claim 21, wherein said oligonucleotides further comprise one or more fixed sequences coupled to said random sequence.

23. The method of claim 22, wherein at least one of said fixed sequences includes at least a portion of a catalytic site for catalyzing a chemical reaction.

24. The method of claim 23, wherein said catalytic site is selected from the group consisting of a ligase site, self-cleaving site, a Group I catalytic site, a Group II catalytic site, and a hammerhead catalytic site.

25. The method of claim 22, wherein at least one of said fixed sequences includes a sequence that facilitates cloning or sequence of said oligonucleotide.

26. The method of claim 25, wherein said sequence is selected from the group consisting of a PCR primer site, an RNA polymerase primer activation site, and a restriction endonuclease recognition site.

27. The method of claim 21, wherein said oligonucleotide is provided on a replicatable nucleic acid sequence.

28. The method of claim 29, wherein said replicatable nucleic acid sequence is a plasmid.

29. The method of claim 23, wherein said random sequence includes a target activation site with the random sequence, wherein said catalytic sequence is activated upon recognition of said target molecule to said target activation site.

30. The method of claim 23, wherein said method further comprises
(i) identifying target-molecule independent catalytic oligonucleotides in said population that have catalytic activity in the absence of said target molecule;
(ii) removing said oligonucleotides from said population prior to contacting said population with said target molecule; and, optionally,
repeating steps (i) and (ii).

31. The method of claim 23, wherein said method comprises
(i) identifying target-molecule dependent catalytic oligonucleotides in said population, wherein said target-molecule dependent catalytic oligonucleotides have catalytic activity upon recognizing said target molecule;
(ii) removing said target-molecule dependent catalytic oligonucleotides from said population of oligonucleotides; and, optionally,
repeating steps (i) and (ii).

32. The method of claim 30, wherein said method comprises
(i) identifying target-molecule dependent catalytic oligonucleotides in said population, wherein said target-molecule dependent catalytic oligonucleotides have catalytic activity upon recognizing said target molecule;
(ii) removing said target-molecule dependent catalytic oligonucleotides from said population of oligonucleotides; and, optionally,
repeating steps (i) and (ii).

33. The method of claim 23, wherein said fixed sequence is a portion of a catalytic site, and said catalytic site is non-functional.

34. The method of claim 33, wherein said oligonucleotide includes a 3′ nucleotide couplable to a first signaling moiety and a 5′ moiety couplable to a second signaling moiety, wherein said first and second signaling moieties change proximity to each other upon recognition of said target molecule by said target activation site.

35. The method of claim 34, wherein said first and second signaling moieties comprise a fluorescent label and a fluorescent quencher, and allosteric modulation of said nucleic acid sensor following recognition of said target molecule results in an increase in detectable fluorescence of said fluorescent label.

36. The method of claim 34, wherein said first signaling moiety and said second signaling moiety comprise fluorescent energy transfer (FRET) donor and acceptor groups, and allosteric modulation of said nucleic acid sensor molecule following recognition of said target molecule results in a change in distance between said donor and acceptor groups, thereby changing optical properties of said molecule.

37. The method of claim 20, wherein said target molecule comprises a polypeptide.

38. The method of claim 37, wherein said polypeptide is a secreted polypeptide.

39. The method of claim 37, wherein said polypeptide is a membrane-associated polypeptide.

40. The method of claim 39, wherein said membrane is a plasma membrane.

41. The method of claim 37, wherein said polypeptide is a cytosolic polypeptide.

42. The method of claim 37, wherein said polypeptide comprises the amino acid sequence of a nuclear hormone receptor (NHR) polypeptide.

43. The method of claim 37, wherein said polypeptide comprises the amino acid sequence of at least a fragment of a G-protein coupled receptor (GPCR) polypeptide.

44. The method of claim 43, wherein said GPCR polypeptide is a human GPCR polypeptide.

45. The method of claim 43, wherein said polypeptide comprises the amino acid sequence of a ligand-binding portion of a GPCR polypeptide.

46. The method of claim 43, wherein said polypeptide comprises the amino acid sequence of a GPCR polypeptide.

47. The polypeptide comprises the amino acid sequence of a phosphodiesterase (PDE) polypeptide.

48. The method of claim 37, wherein the conformation change of said oligonucleotide upon recognizing said PDE polypeptide is dependent on the presence of a cyclic nucleotide.

49. The method of claim 48, wherein said cyclic nucleotide is cAMP.

50. The method of claim 48, wherein said cyclic nucleotide is cGMP.

51. The method of claim 37, wherein said polypeptide is modified polypeptide.

52. The method of claim 37, wherein said polypeptide comprises the amino acid sequence of a phosphodiesterase (PDE) polypeptide or a protein kinase polypeptide (PK).

53. The method of claim 52, wherein said PDE or PK polypeptide is a modified PDE or PK polypeptide.

54. The method of claim 53, wherein said modified PDE polypeptide or PK polypeptide is a phosphorylated PDE polypeptide.

55. The method of claim 53, wherein said oligonucleotide binds with higher affinity to said modified PDE polypeptide or PK polypeptide than to an un-modified PDE or PK polypeptide.

56. An oligonucleotide identified by the method of claim 20.

57. A plurality of nucleic acid sensor molecules, the plurality comprising two or more nucleic acid sensor molecules, said two or more nucleic acid sensor molecules comprising
a target molecule activation site, the target molecule activation site comprising
a structure that recognizes a target molecule and
an optical signaling unit, wherein said optical signaling unit includes at least one nucleotide coupled to a signaling moiety; and wherein said signaling moiety changes its optical properties upon an allosteric modulation of said nucleic acid sensor molecule following recognition of said target molecule.

58. The plurality of claim 57, wherein said optical signaling units in said two or more nucleic acid sensor molecules comprise a first nucleotide coupled to a first signaling moiety and a second nucleotide coupled a second signaling moiety, and wherein said first and second signaling moieties change proximity to each other upon allosteric modulation of said target molecule following recognition by said target activation site of said target molecule.

59. The plurality of claim 58, wherein said first and second signaling moieties comprise a fluorescent label and a fluorescent quencher, and allosteric modulation of said nucleic acid sensor following recognition of said target molecule results in an increase in detectable fluorescence of said fluorescent label.

60. The plurality of claim 59, wherein said first signaling moiety and said second signaling moiety comprise fluorescent energy transfer (FRET) donor and acceptor groups, and allosteric modulation of said nucleic acid sensor molecule following recognition of said target molecule results in a change in distance between said donor and acceptor groups, thereby changing optical properties of said molecule.

61. The plurality of claim 57, wherein said optical signaling unit in said two or more nucleic acid sensor molecules consist essentially of a first signaling moiety, wherein said first signaling moiety changes conformation upon recognition of said target molecule to said target activation site, thereby resulting in a detectable optical signal.

62. The plurality of claim 57, wherein said two or more biosensor molecules are provided in solution.

63. The plurality of claim 57, wherein said two or more biosensor molecules are provided bound to a substrate.

64. The plurality of claim 53, wherein said substrate is glass, silicon, nitrocellulose, nylon, or plastic.

65. The plurality of claim 57, wherein said attachment is covalent.

66. The plurality of claim 57, wherein said attachment is non-covalent.

67. The plurality of claim 57, wherein at least two members of said plurality recognizes different target molecules.

68. A diagnostic system for detecting a target molecule, the diagnostic system comprising
at least one nucleic acid biosensor, the nucleic acid sensor molecule comprising
a target molecule activation site, the target molecule activation site comprising
a structure that recognizes to a target molecule; and
an optical signaling unit, wherein said optical signaling unit includes at least one nucleotide coupled to a signaling moiety; and wherein said signaling moiety changes its optical properties upon allosteric modulation of said nucleic acid sensor molecule following recognition of said target molecule; and
a detector in optical communication with said nucleic acid biosensor, wherein said detector detects changes in the optical properties of said nucleic acid biosensor.

69. The diagnostic system of claim 68, further comprising a light source in optical communication with said biosensor.

70. The diagnostic system of claim 68, further comprising a processor for processing optical signals detected by the detector.

71. The diagnostic system of claim 68, wherein said system comprises a plurality of nucleic acid biosensor molecules, wherein at least two of said biosensor molecules recognize two different target molecules.

72. A kit for detecting a target molecule, the kit comprising
at least one nucleic acid biosensor, the nucleic acid sensor molecule comprising a target molecule activation site, the target molecule activation site comprising a structure that specifically recognizes a target molecule, and wherein said optical signaling unit comprises a first nucleotide coupled to a first signaling moiety and a second nucleotide coupled a second signaling moiety, and wherein said first and second signaling moieties change proximity to each other upon allosteric modulation by said target molecule to** said target activation site;
a reagent for attaching said first signaling moiety;
a reagent for attaching said second signaling moiety; and, optionally,
control target molecules; and, optionally
one or more buffers for analyte detection.

73. A method for identifying a drug compound, the method comprising
identifying a nucleic acid biosensor-based molecule profile of target molecules associated with a disease trait in a patient;
administering a candidate compound to said patient; and
monitoring changes in said profile.

74. The method of claim 73, wherein said profile is compared to the profile of a reference population.

75. The method of claim 73, wherein said reference population is a healthy population.

76. The method of claim 73, wherein said reference population is a diseased population.

77. A method for identifying a drug compound, the method comprising
identifying a plurality of pathway target molecules;
administering a candidate compound to a patient having a disease trait; and
monitoring changes in the structure, level or activity of two or more said plurality of pathway target molecules using a nucleic acid biosensor biomolecule.

78. The method of claim 77, wherein said changes are compared to a reference population.

79. The method of claim 78, wherein said reference population is a healthy population..

80. The method of claim 78, wherein said reference population is a diseased population.
